(19) United States
(12) Patent Application Publication
(10) Pub. No.: US 2002/0042079 A1
(43) Pub. Date: Apr. 11, 2002

(54) METHODS AND AGENTS FOR MEASURING AND CONTROLLING MULTIDRUG RESISTANCE

(76) Inventors: SANFORD M. SIMON, NEW YORK, NY (US); MELVIN S. SCHINDLER, OKEMOS, MI (US)

Correspondence Address: DAVID A JACKSON, KLAUBER & JACKSON, 411 HACKENSACK AVENUE, HACKENSACK, NJ, 07601

(21) Appl. No.: 09/080,739

(22) Filed: May 18, 1998

Related U.S. Application Data
(63) Continuation-in-part of application No. 08/535,955, filed on Sep. 28, 1995, now Pat. No. 5,616,228.
Application No. 08/535,955 is a continuation-in-part of application No. 08/379,875, filed on Jan. 27, 1995, now abandoned.
Application No. 08/379,875 is a continuation of application No. 08/190,336, filed on Feb. 1, 1994, now abandoned.

(51) Int. Cl. A01N 1/02; C12Q 1/00; G01N 33/53; G01N 33/567; G01N 33/574; C12Q 1/02; G01N 31/16; G01N 31/22; G01N 21/76; C12N 5/00; C12N 5/02; C12N 5/08

(52) U.S. Cl. 435/7.1; 435/2; 435/4; 435/29; 435/7.8; 435/7.21; 435/325; 435/366; 435/374; 436/163; 436/172

(57) ABSTRACT

The effect of the pH of intracellular vesicular compartments and intracellular vesicular transport on multidrug resistance (MDR) of tumor cells is examined. The invention comprises in one aspect the treatment of MDR by administering a therapeutically effective amount of a pH modulator and/or a compound that can interfere with the vesicular transport of an intracellular vesicular compartment. Diagnostic utilities are contemplated and extend to drug discovery assays and methods for measuring monitoring the status of the onset or development of MDR, as well as the measurement of intracellular drug accumulation. Therapeutic compositions include a composition comprising a pH modulator alone or in combination with the dose-limited therapeutic agent(s), and a pharmaceutically acceptable excipient, are also contemplated.

### CROSS-REFERENCE TO RELATED APPLICATIONS

This application is a continuation-in-part of co-pending U.S. patent application Ser. No. 08/535,955, filed Sep. 29, 1995 which is a continuation-in-part of U.S. patent application Ser. No. 08/379,875, filed Jan. 27, 1995, now abandoned, which is a continuation of application Ser. No. 08/190,336, filed Feb. 1, 1994, now abandoned.

### GOVERNMENT SUPPORT

The research leading to the present invention was funded in part by Grant No. GM 447005 from the National Institutes of Health. The government may have certain rights in the invention.

### TECHNICAL FIELD OF THE INVENTION

The present invention relates generally to the field of immunology and, more particularly, to the condition known as multidrug resistance (MDR), and concerns the diagnosis and treatment of MDR and the discovery and development of effective pharmaceutical agents and therapies thereagainst.

### BACKGROUND OF THE INVENTION

Chemotherapy takes advantage of the phenomena that tumor cells are ˜5 fold more sensitive to anti-cancer drugs than are healthy cells. This narrow therapeutic window permits the use of cytotoxic agents to destroy malignancies. However, during chemotherapy, tumor cells often lose this sensitivity and become as vulnerable as normal cells. This diminished sensitivity to the original drug also extends to a broad class of other drugs, diverse in their structures and targets. This acquired multidrug resistance (MDR) is a major challenge to successful chemotherapy of malignant tumors.

Different drug-resistant cells over express a variety of membrane proteins including a subunit of a vacuolar H⁻-ATPase [Ma, L et al., Biochem Biophys. Res. Commun., 182:675-681 (1992)], a protein with homology to CFTR [Cole et al., Science, 258:1650-1654 (1992)] and the P-glycoprotein, a 170-180 kD plasma membrane glycoprotein [Gottesman et al., Annu. Rev. Biochem., 62:385-427 (1993)]. The most generally accepted hypothesis for MDR suggests the P-glycoprotein uses ATP to power a molecular pump that removes chemotherapeutic molecules from the cell [Dano et al., Biochem Biophys., 323:466-483 (1973) and reviewed in Gottesman et al., Annu. Rev. Biochem., 62:385-427 (1993)]. This model proposes that chemotherapeutic agents diffuse down a concentration gradient into the cell and that the pump either transports the drugs out of the cytosol or serves as a flippase to expel them from the bilayer [Higgins et al., TIBS, 17:18-21 (1992)].

Over the past decade there have been a number of experiments that suggest that there might be changes of intracellular pH associated with multidrug resistance (MDR) in tumor cells. However, the preponderance of the thinking in this area maintains that a pump theory of drug expulsion predominates and that the change in pH is merely a secondary characteristic. It is toward the elucidation of this phenomenon and the proposal of diagnostic strategies coordinated therewith that the present invention is directed.

### SUMMARY OF THE INVENTION

The present invention is partially based of the discovery demonstrated herein that the shifts of pH in intracellular vesicular compartments and/or corrections of defects in the intracellular vesicular transport mechanism that occur during multidrug resistance (MDR) are sufficient to produce a decrease in cellular drug accumulation.

In particular, the present invention demonstrates that: (1) the drug-sensitivity of tumor cells can be the consequence of a defect in one or more components of the exocytic apparatus; (2) that this defect is “normalized” in drug-resistant cells; (3) that treatments that reverse MDR also disrupt the secretory pathway; and (4) that any manipulations that selectively disrupt and/or alkalinize exocytic compartments of MDR cells will reverse MDR.

Accordingly, the present invention contemplates methods for the discovery of drugs useful in the modulation of pH and intracellular vesicular transport, and the consequent control of MDR, and extends to the pharmaceutical compositions and corresponding therapeutic methods for their use. Accordingly the present invention provides for the treatment of MDR by administering a therapeutically effective amount of a pH modulator and/or a compound that can interfere with the vesicular transport of an intracellular vesicular compartment.

The underlying investigations presented herein describe how these factors can account for the effects of pH on chemotherapeutic agents that are affected by MDR. The work also is consistent with the observed mechanisms of MDR that are not dependent upon the expression of p-glycoprotein. Finally, assays are proposed and performed herein to select for drugs and treatments that will resensitize MDR cells to chemotherapeutic agents.

One aspect of the present invention provides a method for measuring the development or onset of multidrug resistance in a tumor cell in which such multidrug resistance is suspected, comprising determining whether there is a defect in the vesicular transport mechanism of an intracellular vesicular compartment of the cell, wherein such a defect is symptomatic of the tumor cell being drug-sensitive and the absence of the defect is indicative of the onset or development of multidrug resistance in the tumor cell. In one such embodiment the intracellular compartment of the cell is a secretory compartment. In a particular embodiment the secretory compartment is a perinuclear recycling compartment (PRC). In another embodiment the secretory compartment is a recycling endosome. In yet another embodiment the secretory compartment is a secretory vesicle. In still another embodiment the secretory compartment is the trans-Golgi network (TGN). Tissues of origin for the cells include but are not limited to the brain, lung, breast, colon, and epithelium.

In a preferred embodiment of this method, determining whether there is a defect in the vesicular transport mechanism is performed by measuring the transport of a marker from the intracellular vesicular compartment to the exterior of the cell or the cell surface. In one such embodiment, the marker is a labeled protein. In a preferred embodiment of this type the labeled protein is labeled transferrin. In another embodiment the marker is a labeled lipid. In a preferred embodiment of this type the labeled lipid is labeled sphingomyelin. The marker may be intrinsically detectable (e.g., fluorescent) or be a molecule that is associated with a detectable label which is either adsorbed or bound (either covalently or otherwise) to the molecule and/or to the intracellular vesicular compartment.

Markers used for determining whether there is a defect in the vesicular transport mechanisms of the present invention can be capable of being measured by any appropriate means of detection. In one such embodiment of the above method the marker is detectable by spectrophotometry. In another embodiment the marker is detectable by spectrofluorometry. In a preferred embodiment of this type of the method, a marker that is capable of being measured spectrofluorometrically, is measured by fluorescence microscopy. In an alternative embodiment, a marker that is capable of being measured spectrofluorometrically is measured by confocal microscopy. In another embodiment of the method, the marker is detectable by luminescence. In yet an alternative embodiment, the marker is capable of being detected by reflectance. In another embodiment, the marker is detectable by electron microscopy. In still another embodiment of the method the marker is detectable by its being radioactive.

Markers used for determining whether there is a defect in the vesicular transport mechanisms of the present invention can alternatively be capable of being measured through a biological activity. In one embodiment of the above method, the biological activity is measured by determining the activity on the surface of the cell. In another embodiment, the biological activity is measured by determining the activity on the outside of the cell. In still another such embodiment, the biological activity is measured by determining the activity from the inside of the cell.

The present invention further provides a method for screening potential drugs to treat multidrug resistant by identifying a candidate drug that decreases vesicular transport in a multidrug resistant tumor cell. One such embodiment comprises contacting a multidrug resistant tumor cell with a potential drug wherein the multidrug resistant cell comprises an intracellular vesicular compartment that contains a marker; and measuring the transport of the marker out of the intracellular vesicular compartment. A potential drug is identified as a candidate drug if the transport of the marker out of the intracellular vesicular compartment of the multidrug resistant tumor cell decreases. In preferred embodiments of this method, the cell is a mammalian cell. Appropriate cells include those obtained from the American Type Culture Collection such as uterine sarcoma cells, leukemia cells, colorectal carcinoma cells, mammary cells (as exemplified below), and neuroblastoma drug-resistant cells

In one embodiment of this method a plurality of potential drugs are tested at a plurality of drug concentrations. In another embodiment, measuring the transport of the marker from the intracellular vesicular compartment is performed by measuring the rate of transport of the marker from the intracellular compartment of the cell to the exterior of the cell or the cell surface. In one embodiment, the marker is a labeled protein. In a preferred embodiment of this type the labeled protein is labeled transferrin. In another embodiment the marker is a labeled lipid. In a preferred embodiment of this type the labeled lipid is labeled sphingomyelin. The marker may be intrinsically detectable (e.g., fluorescent) or be a molecule that is associated with a detectable label which is either adsorbed or bound (either covalently or otherwise) to the molecule and/or to the intracellular vesicular compartment.

The marker of the method can be capable of being measured by any appropriate means of detection. In one such embodiment the marker is detectable by spectrophotometry. In another embodiment the marker is detectable by spectrofluorometry. In a preferred embodiment of this type of the method, a marker that is capable of being measured spectrofluorometrically, is measured by fluorescence microscopy. In an alternative embodiment, a marker that is capable of being measured spectrofluorometrically is measured by confocal microscopy. In another embodiment of the method, the marker is detectable by luminescence. In yet an alternative embodiment, the marker is capable of being detected by reflectance. In another embodiment, the marker is detectable by electron microscopy. In still another embodiment of the method the marker is detectable by its being radioactive.

The marker used in this method can alternatively be capable of being measured through a biological activity. In one such embodiment of the method, the biological activity is measured by determining the activity on the surface of the cell. In another embodiment, the biological activity is measured by determining the activity on the outside of the cell. In still another such embodiment, the biological activity is measured by determining the activity from the inside of the cell.

The present invention further includes assay systems for screening a potential drug for the treatment of multidrug resistance (MDR). One such embodiment comprises a multidrug resistant tumor cell and a marker that can be used for measuring the transport to the cell surface from the intracellular compartment of the cell. In a preferred embodiment the tumor cell is a mammalian tumor cell. In one embodiment of this type, the mammalian cell is a human cell.

Another aspect of the present invention is a method for treating multidrug resistance in an animal (preferably a mammal, and more preferably a human), containing a multidrug resistant tumor cell comprising administering to the animal a drug that decreases the rate of transport of an intracellular vesicular compartment of the multidrug resistant tumor cell in an amount effective to decrease the rate of transport and therein increase the drug sensitivity of the tumor cell. In one embodiment of this type the drug is administered in association with the administration of a chemotherapeutic agent already under administration to the tumor cell. In another such embodiment, the drug is administered simultaneously with the chemotherapeutic agent. In still another such embodiment the drug is administered in a pharmaceutical composition comprising the drug and said chemotherapeutic agent. The drug can be administered in any fashion including parenterally or orally.

The present invention further provides a therapeutic composition for the treatment of multidrug resistance in an animal (preferably a mammal, and more preferably a human) comprising, in unit dose form, a drug that decreases the rate of transport of an intracellular vesicular compartment of the multidrug resistant tumor cell, and a pharmaceutically acceptable excipient. In one embodiment the composition includes a chemotherapeutic agent to which the animal has developed multidrug resistance to.

Yet another aspect of the present invention includes methods for measuring the development or onset of pH-dependent multidrug resistance in a tumor cell in which such multidrug resistance is suspected. One such embodiment comprises determining whether there is a defect in the acidification of an intracellular vesicular compartment of the cell, wherein the defect is symptomatic of the tumor cell being drug-sensitive, and wherein the absence of the defect is indicative of the onset or development of multidrug resistance in the tumor cell. In one such embodiment the intracellular vesicular compartment of the cell is a lysosome. In another embodiment the intracellular vesicular compartment of the cell is a secretory compartment. In a particular embodiment of this type, the secretory compartment is a perinuclear recycling compartment (PRC). In another embodiment the secretory compartment is a recycling endosome. In yet another embodiment the secretory compartment is a secretory vesicle. In still another embodiment the secretory compartment is the trans-Golgi network (TGN). Tissues of origin of the cells include but are not limited to the brain, lung, breast, colon, and epithelium.

In one such embodiment of the method, the measure of the pH is determined by directly measuring the pH in the intracellular vesicular compartment. In one embodiment of this type, the pH is measured with a pH electrode. In another embodiment of this type, the pH is measured with a pH sensitive probe. In a preferred embodiment of this type the pH probe is targeted for a specific intracellula r vesicular compartment. In one such embodiment, the pH probe is targeted to the endosomes. In a preferred embodiment the pH probe is targeted to the endosomes by being associated with transferrin. In a related embodiment, the pH probe is targeted to the Golgi. In a preferred embodiment the pH probe is targeted to the Golgi by being associated with verotoxin.

In an alternative embodiment the measure of the pH is determined indirectly by assaying for a detectable consequence of a defect in the acidification of an intracellular vesicular compartment. In one such embodiment the consequence of a defect in the acidification of an intracellular vesicular compartment is a decrease in the glycosylation of lipids on the surface of the cell. In a particular embodiment of this type, the decrease in the glycosylation of the lipids on the surface of the cell is identified by a decrease of sialic acids attached to lipids. In a related embodiment the consequence of a defect in the acidification of an intracellular vesicular compartment is a decrease in the glycosylation of proteins on the surface of the cell. In a particular embodiment of this type, the decrease in the glycosylation of the proteins on the surface of the cell is identified by a decrease of sialic acids attached to proteins. In still another embodiment the consequence of a defect in the acidification of an intracellular vesicular compartment is an increase in the secretion of lysosomal enzymes from the cell. In all of these cases, the defect is associated with a drug sensitive tumor cell, and the lack of a defect (or correction of the defect) is associated with a multidrug resistant tumor cell (or a wild type cell non-tumorous cell).

In still another embodiment of the method for measuring the development or onset of pH-dependent multidrug resistance in a tumor cell in which such multidrug resistance is suspected, the intracellular vesicular compartment of the tumor cell is infiltrated with a pH indicator prior to determining the pH. In one such embodiment the pH indicator is acridine orange. In one such embodiment the pH indicator is Lysosensor Blue DND-167. In another embodiment the pH indicator is SNARF. In still another embodiment the pH indicator is SNAFL. In yet another embodiment the pH indicator is FITC. In still another such embodiment the pH indicator is BCECF. In yet another embodiment the pH indicator is DAMP.

The pH indicators of the present invention can be capable of being measured by any appropriate means of detection. In one such embodiment the pH indicator is detectable by spectrophotometry. In another embodiment the pH indicator is detectable by spectrofluorometry. In a preferred embodiment of this type of the method, a pH indicator that is capable of being measured spectrofluorometrically, is measured by fluorescence microscopy. In an alternative embodiment, a pH indicator that is capable of being measured spectrofluorometrically is measured by confocal microscopy. In another embodiment of the method, the pH indicator is detectable by luminescence. In still another embodiment of the method the pH indicator is detectable by radioactivity. In yet another embodiment of the method the pH indicator is detectable by electron microscopy.

The present invention further provides methods for screening potential drugs to identify candidate drugs for treating pH-dependent multidrug resistance in animals preferably mammals and more preferably humans. One such embodiment comprises contacting a multidrug resistant tumor cell with a potential drug, wherein prior to said contacting it is determined that there is a no defect in the acidification of an intracellular vesicular compartment of the cell. Next it is determined whether a defect in the acidification of the intracellular vesicular compartment of the tumor cell is present, wherein the defect is symptomatic of the tumor cell being drug-sensitive . The determination of the defect in the presence of the potential drug identifies the potential drug as a candidate drug for the treatment of multidrug resistance. In one such embodiment, the determination of whether a defect in the acidification of the intracellular vesicular compartment of the tumor cell is present is made by determining whether the potential drug increases the pH of the intracellular vesicular compartment. In this case, if the potential drug increases this pH, the potential drug is identified as a candidate drug for the treatment of multidrug resistance.

Appropriate cells for this method include those obtained from the American Type Culture Collection such as uterine sarcoma cells, leukemia cells, colorectal carcinoma cells, mammary cells (as exemplified below), and neuroblastoma drug-resistant cells. Tissues of origin can include but are not limited to the brain, lung, breast, colon, and epithelium.

In one embodiment of the method an intracellular vesicular compartment of the tumor cell is infiltrated with a pH indicator. In another such embodiment a plurality of potential drugs are tested at a plurality of drug concentrations.

In a particular embodiment the method can further comprise contacting a non-tumorous cell with the candidate drug, wherein prior to the contacting it is determined that there is no defect in the acidification of an intracellular vesicular compartment of the non-tumorous cell. Next it is determined whether the acidification of the intracellular vesicular compartment of the non-tumorous cell is altered. The lack of an alteration in the acidification of the intracellular vesicular compartment of the non-tumorous cell confirms the identification of the candidate drug.

The present invention further provides an assay system for screening a potential drug for the treatment of pH-dependent multidrug resistance (MDR) in animals, preferably mammals and more preferably humans which comprises a tumor cell susceptible to or experiencing MDR, and a pH indicator of the present invention that can be placed into an intracellular vesicular compartment of the tumor cell.

The present invention also includes methods for treating pH-dependent multidrug resistance in a tumor cell comprising administering to the tumor cell a pH modulator (or agent) in an amount effective for disrupting the acidification of an intracellular vesicular compartment of the tumor cell and thereby alleviating the multidrug resistance in the tumor cell.

In one embodiment of this type the pH modulator (or agent) is administered in association with the administration of a chemotherapeutic agent already under administration to the tumor cell. In another such embodiment, the pH modulator (or agent) is administered simultaneously with the chemotherapeutic agent. In still another such embodiment the pH modulator (or agent) is administered in a pharmaceutical composition comprising the drug and said chemotherapeutic agent. The pH modulator (or agent) can be administered in any fashion including parenterally or orally.

The present invention further provides a therapeutic composition for the treatment of multidrug resistance in an animal (preferably a mammal, and more preferably a human) comprising, in unit dose form, a drug that is a modulator of the pH of an intracellular vesicular compartment of the multidrug resistant tumor cell, and a pharmaceutically acceptable excipient. In one embodiment the composition includes a chemotherapeutic agent to which the animal has developed multidrug resistance to.

The invention also extends to methods and corresponding kits for measuring defects in the acidification of the intracellular vesicular compartments and/or defects in the transport of intracellular vesicular compartments in cells, and consequently measuring the onset or likelihood of occurrence of multidrug resistance. The methods also include the screening of drugs and other agents capable of effecting these defects in MDR tumor cells in vivo so as to counteract MDR to a degree sufficient to resensitize target cells such as neoplastic tumor cells, to effective treatment with chemotherapeutic agents.

Accordingly, it is a principal object of the present invention to provide a methods for preventing the development of multidrug resistance (MDR) in mammals.

It is a further object of the present invention to provide methods for the assessing the onset of MDR.

It is a further object of the present invention to provide a method and associated assay system for screening substances such as drugs, agents and the like, potentially effective in preventing or treating MDR in mammals.

It is a still further object of the present invention to provide pharmaceutical compositions for use in the prevention or treatment of MDR in mammals.

Other objects and advantages will become apparent to those skilled in the art from a review of the ensuing description which proceeds with reference to the following illustrative drawings.

### BRIEF DESCRIPTION OF THE DRAWINGS

FIG. 1 shows the subcellular localization of daunomycin and doxorubicin. A fluorescence photomicrograph of the daunomycin fluorescence in NIH3T3 fibroblasts demonstrating the subcellular localization of daunomycin and doxorubicin. Daunomycin (5 μM) was added to the medium 45 min. prior to viewing with conventional fluorescence microscopy.

FIGS. 2a and 2b are plots, demonstrating the effect of pCO₂ on cytosolic pH: In FIG. 2a. Myeloma cells were loaded with SNARF1 and the pCO₂ in the medium was shifted between 2% (dashed lines) and 5% (solid line). The fluorescence emission was recorded between 520 nm and 700 nm using an excitation of 514 nm for both the drug-sensitive (8226 black line) or resistant cells (DOX 40 grey line). The pH (as indicated by the ratio of the emission at 630 nm to 585 nm) is indistinguishable between the sensitive cells in 2% pCO₂ (dashed black line) and the resistant cells in 5% pCO₂ (solid grey line). The myeloma cells were loaded with the pH sensitive dye SNARF1 for 15 minutes at 37° C. while grown 10 RPM1 without FCS. In FIG. 2b, the pH is plotted for both the drug-sensitive cells (white) and resistant cells (grey) at ambient, 0.03%, 2%, 5% and 10% pCO2.

FIG. 3a demonstrates the effect of shifting pCO₂ on the daunomycin fluorescence in NIH3T3 cells. Fibroblasts were incubated in 2 μM daunomycin and excited at 488 nm and emission recorded at 570 nm every 15 sec. The medium initially was equilibrated with 5% pCO₂ (The first 7 frames—red background). The pCO₂ was shifted to 2% for 2 min. (blue background) and there was a substantial decrease in the cellular daunomycin fluorescence. Upon returning to 5% pCO₂ (red background) the cellular daunomycin fluorescence returned. The cells were repeatedly cycled between 5% pCO₂ (red background) and 2% pCO₂ (blue background). The daunomycin concentration is pseudocolored with the lowest level in black and increasing concentrations in blue, green, red and yellow.

FIG. 3b demonstrates the quantification of the effect of pCO2 on daunomycin fluorescence in NIH3T3 cells (from FIG. 3a) The daunomycin fluorescence was quantified for six different cells as the pCO₂ was shifted between 2 and 5%. Reducing the pCO₂ raises the cytosolic pH and reduces the cell-associated daunomycin fluorescence. These effects are completely reversible and can be repeated on the same cells many times.

FIG. 4 demonstrates the effect of shifting pCO₂ on the daunomycin fluorescence in myeloma cells. Myeloma cells (8226) were grown in suspension in RPMI at pCO₂ of 5%. Cells were attached to cover glass slips covered with Cell-Tak and then mounted in a Leiden cover glass chamber. After incubating the cells in 6 μM daunomycin for 40 min. the cells were monitored under standard fluorescence microscopy. The pCO₂ perfusing the surface of the chamber was consecutively switched for 4 minute Intervals from 5% to 2%, to ambient (˜0.033%), back to 2%, 5%, 10% and then returned to 5%. The cycle was then repeated. Increasing the pCO₂, which acidifies the cytosol, increased the cell-associated daunomycin fluorescence. The cell-associated daunomycin fluorescence was correlated with pCO₂ and inversely correlated with pH. The daunomycin concentration is pseudocolor coded with the lowest value in green and increasing levels in orange, red and yellow.

FIG. 5a-5d shows Acridine orange staining of MCF-7 and MCF-7adr cells. Acridine orange, a label for the acidic compartments, labels (FIG. 5a) the drug-sensitive human breast cancer cells (MCF-7) labels weakly in contrast to (FIG. 5b) the labeling of the adriamycin resistant variant (MCF-7adr). Treatment of both cell lines with nigericin (7.5 μM) at room temperature resulted in the immediate loss of orange/red fluorescence within (FIG. 5c) MCF-7 and (FIG. 5d) MCF-7adr cells. It is interesting to note that although the pericentriolar region in MCF-7adr cells no longer stains red with acridine orange following treatment with nigericin, it remains visible as an unstained area with a green fluorescent background (FIG. 5d). This suggests that it is the pH gradient between the pericentriolar compartment/vesicles and the cytoplasm that is altered not its integrity during the period of examination. Methods: Cells were seeded and grown in Dulbecco Modified Eagle's (DME) media containing 10% fetal calf serum (no phenol red) in Lab-Ten culture chambers (Nunc, Naperville, Ill.) maintained in an incubator at 37° C. and 5% CO₂. The media for the MCF-7adr cells was supplemented with adriamycin (0.5 μg/ml). Cells were utilized 3-4 days following plating. Acridine orange (2 μg/ml media; 4 mg/ml stock (in water) was added directly to the chambers and the cells were incubated with the dye at 37° C. for 30 minutes. Cells in the presence of acridine orange were then examined at room temperature with an Insight Bilateral Laser Scanning Confocal Microscope (Meridian Instruments, Okemos, Mich.). Excitation was at 488 nm (argon ion laser beam) and dual emission confocal images were sequentially recorded utilizing both a 530-30 band pass barrier filter (green fluorescence) and a 605 nm long pass barrier filter (red fluorescence). Acridine orange demonstrates a concentration dependent long wavelength shift in the fluorescence emission and shows a red fluorescence when accumulated to a high concentration within cellular compartments (acidic) and a green fluorescence when bound at lower concentration to membranes and/or nucleic acids. Optical sections of the fluorescent sample were recorded at 0.5 micron intervals. Typical individual sections are presented to demonstrate the distribution of acridine orange within the cytoplasmic and vesicular compartments. Human breast cancer cells (MCF-7) and the adriamycin resistant line (MCF-7adr) were obtained from Dr. William W. Wells of the Dept. of Biochemistry, Michigan State University.

FIG. 6a-6c show Measurements of Intravesicular pH utilizing SNAFL-calcein. FIG. 6a shows gradients of intracellular pH are relatively absent from MCF7 cells as assayed by fluorescence of SNAFL-calcein. In contrast, significant pH gradients, including an acidic pericentriolar labeling is observed in MCF7adr cells (FIG. 6b). The intravesicular pH in drug-resistant MCF-7adr cells (white bars) is more alkaline than the vesicular pH of the drug-sensitive parental MCF-7 (black bars) (FIG. 6c). This acidic pH difference is reduced by treatment of MCF-7adr cells with monensin (dark grey). Methods: The acetoxymethylester derivative of SNAFL-calcein (15 μg/ml) (a radiometric fluorescent probe for pH) was added to both MCF-7 and MCF-7adr cells. The ester linked fluorescent probe enters the cell passively where the esters are hydrolyzed by esterases located in the cytoplasm and intracellular vesicles. The SNAFL-calcein is then ironically trapped within the cytoplasm and vesicular compartments. The cells were incubated at 37° C. for 45 minutes and then examined in with the Insight confocal fluorescence. Optical sections were obtained utilizing two different filter settings for emission (530-30 band pass barrier filter and 630 long pass filter) and a single excitation wavelength (488 nm) as previously described for carboxy SNARF-1. The pixel intensities obtained at the two different emission intensities were then divided to obtain a ratio image of the internalized pH probe. These images were then compared to standard curves that were obtained in the following manner. To obtain a quantitative relationship between emission ratios and pH, each SNAFL-calcein stained cell line was exposed to a buffer at a known pH containing nigericin/high K⁺ (18 μM/150 mM KCl). This treatment equilibrates all the internal compartments of the cell to the pH buffer of the incubating buffer. By sequentially changing the pH buffer that is bathing the cells, a pH curve was generated for each cell line that demonstrated the relationship between the SNAFL-calcein fluorescence emission ratio and pH. These values were then incorporated into a pH imaging routine that provides a direct read-out of pH values for individual intracellular compartments that are queried on the computer screen. Differences in distribution and fluorescent intensity of SNAFL-calcein are observed following labeling of (a) MCF-7 and (b) MCF-7adr cells. (c) To prepare histograms of vesicular pH, 5 vesicles within a confocal section were analyzed/cell (20 cells per cell type). Cells treated with monensin were exposed to the drug (10 μg/ml of media) for 30 minutes at 37° C. prior to labeling with SNAFL-calcein as described above. All cells were examined at room temperature.

FIG. 7a-7f shows the Fluorescent labeling of the TGN and secretory vesicles with Bodipy-Ceramide. FIG. 7a shows the Golgi of MCF-7 cells is diffuse throughout the cytoplasm and, as observed in the enlargement (FIG. 7b), is in part vesicular and part cisternal, interconnected via fine tubules. In contrast FIG. 7c shows the Golgi of MCF-7adr cells is compact and pericentriolar and as observed in the enlargement, (FIG. 7d) small secretory vesicles are observed in the cytosol. FIG. 7c shows the diffuse distribution of endocytosed vesicles containing internalized bodipy lactalbumin in MCF-7 cells is different from the compact pericentriolar localization observed in the drug-resistant MCF-7adr cells (FIG. 7f). Methods: Bodipy-ceramide (Bodipy-Cer; Molecular Probes, Eugene, Oreg.) has been demonstrated to label Golgi membranes [Zunino et al. Chem. Biol. Interact. 24, 217-225 (1979)]. Conversion of Bodipy-Cer to Bodipy-sphingomyelin (in cis Golgi) results in the movement of this fluorescent lipid to the trans-Golgi network (TGN). As the Bodipy-sphingomyelin concentration increases within the TGN and secretory vesicles, a long wavelength shift in fluorescence occurs results in red fluorescent structures (TGN and secretory vesicles) against a green fluorescent background. Cells were incubated with Bodipy-Cer (3 μg/ml) for 15 minutes at 37° C., washed once with fresh media and then examined in optical section at room temperature with confocal fluorescence microscopy. Excitation was at 488 nm and dual emission images were prepared utilizing the filter set described for acridine orange (FIG. 1). To examine internalization, Bodipy-lactalbumin (Bodipy-Lac, Molecular Probes, Eurgene, Oreg.) was used as a fluid phase marker. Cells were incubated with Bodipy-Law (2 mg/ml) for 90 minutes at 37° and then washed once with cold media and rapidly examined with confocal fluorescence microscopy (λₑₓ 488 nm, λₑₘ530-30 nm band pass filter). The images are as follows: (FIG. 7a) Bodipy-Cer labeling of MCF-7 cells, enlarged view of (FIG. 7b) showing tethered vesicles within MCF-cells (FIG. 7c) Bodipy-Cer labeling of MCF-7adr cells (FIG. 7d) Bodipy-Lac labeling of MCF-7 cells (FIG. 7e) and Bodipy-Lac labeling of MCF-7adr cells (FIG. 7f).

FIG. 8 shows the intracellular redistribution of adriamycin and the disruption of the TGN and PCR in drug resistant human breast cancer cells (MCF-7adr) following treatment with tamoxifen. Cells were seeded and grown in Dulbecco Modified Eagle's media containing 10% fetal calf serum (phenol red free) in Lab-Tek culture chambers (Nunc, Naperville, Ill.) maintained in an incubator at 37° C. and 5% CO₂. Human breast cancer cells (MCF-7) and the adriamycin resistant line (MCF-7adr) were obtained from Dr. William W. Wells of the Dept. of Biochemistry, Michigan State U. The media for the MCF-7adr cells was supplemented with adriamycin (0.5 μg/ml). Cells were utilized 3-4 days following plating. Unless otherwise indicated all cells were labeled at 37° C. and then examined at room temperature in optical sections with an Insight Bilateral Laser Scanning Confocal Microscope (Meridian Instruments, Okemos, Mich.).

Distribution of adriamycin (top row): Adriamycin (5 μg/ml) (Calbiochem, La Jolla, Calif.) distribution was examined following a 30 min. incubation with the drug at 37° C. in 5% CO₂. in the absence or presence of tamoxifen (50 μM treatment for 20 min. at 37° C. and 5% CO₂). Confocal fluorescence microscopy was performed with excitation at 488 nm (argon ion laser). MCF-7adr cells show a pericentriolar distribution of adriamycin (left) that changes to an intranuclear distribution following treatment with tamoxifen (50 μM (Sigma, St. Louis, Mo.) (middle). This nuclear pattern of adriamycin labeling is similar to that observed within drug sensitive MCF-7 cells (right).

Acidic compartments (second row): Acridine orange demonstrates a concentration dependent long wavelength shift in the fluorescence emission and shows a red fluorescence when accumulated to a high concentration within cellular compartments (acidic) and a green fluorescence when bound at lower concentration to membranes and/or nucleic acids. To examine intracellular acidic compartments, acridine orange (2 μg/ml media; 4 mg/ml stock (in water), Aldrich, Milwaukee, Wis.) was added directly to the chambers and the cells were incubated for 30 minutes. Cells in the presence of acridine orange were then examined utilizing an excitation at 488 nm and dual emission confocal images were sequentially recorded utilizing both a 530-30 band pass barrier filter (green fluorescence) and a 605 nm long pass barrier filter (red fluorescence). Optical sections of the fluorescent sample were recorded at 0.5 micron intervals. Typical individual sections are presented to demonstrate the distribution of acridine orange. MCF-7adr show a pericentriolar labeling (left) that disappears following treatment with tamoxifen (middle). Pericentriolar labeling is also absent in drug sensitive MCF-7 cells (right).

Trans-Golgi network (third row): Bodipy-ceramide (Bodipy-Cer; Molecular Probes, Eugene, Oreg.) has been demonstrated to label Golgi compartments (11). Cells were incubated with Bodipy-Cer (3 μg/ml) for 15 minutes at 37° C., washed once with fresh media and then examined in optical sections. Excitation was at 488 nm and dual emission images were prepared utilizing the filter set described for acridine orange. A tight pericentriolar pattern of labeling is observed within MCF-7adr cells for Bodipy-Cer (left). This is disrupted following treatment with tamoxifen (middle) and is similar to that observed for the drug sensitive MCF-7 cells (right)

Endocytotic pathway (fourth row): To examine internalization, Bodipy-lactalbumin (Bodipy-Lac, Molecular Probes, Eugene, Oreg.) was used as a fluid phase marker. Cells were incubated with Bodipy-Lac (2 mg/ml) for 90 minutes at 37° C. and then washed once with cold media and rapidly examined with confocal fluorescence microscopy ((ex=488 nm, (em 530-30 nm band pass filter). Excitation and emission wavelengths were as described for adriamycin. Bodipy-Lac is also observed to be concentrated within vesicles associated with a pericentriolar compartment in MCF-7adr cells (left). Bodipy-Lac staining following tamoxifen treatment is more punctate and diffuse within the cytoplasm with no localization to the pericentriolar region (middle) similar to its distribution in MCF-7 cells (right).

FIG. 9 shows adriamycin sensitivity studies in the absence and presence of tamoxifen. Cell viability assays were performed in the following manner: the media was removed 60 hours after plating the cells and replaced with fresh media supplemented with various concentrations of adriamycin (Calbiochem, Calif.) and tamoxifen (solubilized in DMF 0.1%) (Sigma, St. Louis). After 6 hours, the media was removed, the cells rinsed, and then fed with fresh media not containing drugs. The cells were fed daily for three days and then the DNA content of the adherent cells was quantified fluorometrically by Hoechst 33258 fluorescence. Media was aspirated and the wells rinsed with Hanks Balanced Salt Solution (HBSS, phenol red free). The cells were sonicated in hypotonic media (0.1×HBSS) for 30 seconds. The homogenate from each well was collected and Hoechst 33258 was added to a final concentration of 1 μg/ml. Fluorescence was measured on an SLM Aminco-Bowman series 2 luminescence spectrometer with a λₑₓ of 356 nm and a λₑₘ of 492 nm. Calf thymus DNA was used for calibration.

FIGS. 10a-10b shows the adriamycin distribution between drug-resistant and drug-sensitive MCF-7 cells. FIG. 10a shows that in MCF-7/adr cells the Adriamycin is excluded from the nucleus. It is concentrated in punctate organelles throughout the cytoplasm and a brightly fluorescent region immediately adjacent to the nucleus. This perinuclear labeling is typical for the recycling endosomes and trans-Golgi network. FIG. 10b shows that in MCF-7 cells the fluorescence of Adriamycin is observed to be diffusely localized throughout the cytoplasm and nucleoplasm. There is very little accumulation in any subcompartment in the cytoplasm. Adriamycin is also seen labeling the nuclear envelope. Adriamycin fluorescence could be due to accumulation in the nuclear envelope or alternatively to binding to the adjacent euchromatin. Cells were incubated in the presence of 10 μM Adriamycin as described in Example 4. After 30 minutes the cells were examined under confocal microscopy with an excitation of 488 nm and emission collected at >600 nm. The scale bar 5 μM.

FIGS. 11a-11i shows the double labeling of Adriamycin and the perinuclear recycling compartment, trans-Golgi Network, and highly acidified organelles. To characterize the regions that accumulated the Adriamycin in MCF-7/ADR cells, BODIPY-transferrin was used to label the recycling endosomal compartment, NBD-ceramide was used to label the trans-Golgi network (TGN), and Lysosensor Blue was used to label the lysosomes. All images were taken using a confocal microsope. FIG. 11a shows that MCF-7 cells accumulate very low levels of Lysosensor Blue indicating the lack of highly acidified organelles. FIG. 11b shows that in MCF-7/ADR cells, Lysosensor Blue labels many large, punctate peripheral organelles consistent with lysosomes. The arrows indicate a group of six lysosomes that co-label with adriamycin in FIG. 11c. The same cell was subsequently labeled with 10 μM Adriamycin (FIG. 11c). Arrow shows four lysosomes that co-label with Lysosensor Blue and Adriamycin. Note that the perinuclear compartment, though highly labeled with Adriamycin, has few lysosomes. FIG. 11d shows that BODIPY-transferrin labels the recycling endosome compartment which is diffuse and punctate in the cytoplasm of MCF-7 cells. Note that the distribution of this compartment is not polarized to any one region of the cytoplasm. FIG. 11e shows that BODIPY-transferrin labels the recycling endosome compartment which is tightly perinuclear in MCF-7/ADR cells. Note that the compartment is polarized to one side of the nucleus. Subsequent labeling of the same cells with Adriamycin also localizes in a perinuclear compartment (FIG. 11f) which overlaps the compartment labeled in (FIG. 11e). FIG. 11g shows NBD-ceramide labeling of the TGN which in MCF-7 cells are stacks distributed in a non-uniform fashion throughout the cytoplasm. In some cells the TGN is perinuclear but not polarized to one side of the nucleus. FIG. 11h shows NBD-ceramide labeling of the TGN in MCF-7/ADR cells. In contrast to MCF-7 cells, this compartment is tightly positioned to one side of the nucleus. Subsequent labeling of the same cells with Adriamycin also localizes in a perinuclear compartment (FIG. 11I) which overlaps with the TGN compartment labeled in FIG. 11H. Cells were either optically sectioned in 0.2 μm slices and optical sections at equivalent distances through the cell were compared, or the images were taken at a single focus which remained unchanged throughout the course of the experiments. For labeling of the recycling compartment, the cells were incubated with 25 μg/ml of BODIPY transferrin at 37° C. Then they were rinsed as described in the methods and viewed under the confocal microscope. The cells were excited at 488 nm and the emission was collected at 520 nm. For TGN labeling, the cells were incubated at 4° C. with 5 μm N.D.-ceramide for 10 minutes. They were then washed and incubated at 37° C. for another 30 minutes before being visualized using the confocal microscope. The cells were excited at 488 nm and the emission was collected at 520 nm. For Lysosensor Blue labeling, the cells were initially incubated with 2 μM Lysosensor Blue DND 167 at 37° C. for 60 minutes. Then they were excited with 353 nm light. The emission was collected at 430 nm. Subsequently the same cells were incubated with 10 μM Adriamycin for 20 minutes. The cells were subsequently excited with 488 nm light, the emission >600 nm was collected. The scale bar is 10 μm.

FIGS. 12a-12b show the distribution of lysosomes in MCF-7 and MCF-7adr cells. LAMP-1 is a membrane protein of lysosomes. LAMP-1 is found in punctate organelles throughout the cytoplasm of MCF-7/ADR cells (FIG. 12a) and (FIG. 12b) MCF-7 cells. Analysis of large fields of MCF-7 and MCF-7/ADR cells did not reveal any significant differences in the phenotypic distribution and number of lysosomes per cell. MCF-7/ADR (FIG. 12a) and MCF-7 (FIG. 12b) cells were fixed in paraformaldehyde, permeabilized with saponin, and labeled with an antibody against the LAMP-1 protein, a membrane marker for lysosomes as described in the methods. The distribution of the LAMP-1 antibody was assayed using a secondary fluorescent antibody and visualized using a laser-scanning confocal microscopy. The scale bar is 10 μm.

FIG. 13 shows the chemical structures of four widely used chemotherapeutics. Adriamycin and Daunomycin belong to the anthracycline class of compounds, vincristine and vinblastine are representative of the Vinca alkaloids. Note that these drugs all are weak bases with pK's between 7.2-8.4 and they are all partially hydrophobic and partially hydrophilic. This property allows them to diffuse across lipid bilayers.

FIG. 14a-14b shows that there is a lack of acidification within the subcellular compartments of drug-sensitive MCF-7 cells as assayed by acridine orange. Cells were incubated with 6 μM acridine orange for fifteen minutes. The cells in FIGS. 14a and 14b were observed using confocal microscopy at 37° C. and 5% pCO₂ whereas the cells in FIG. 14b were observed under epifluorescence. Acridine orange is a weak base that accumulates in acidic compartments. At higher concentrations there is a quenching of the fluorescence in the green part of the spectrum resulting in a shift to red-orange fluorescence. FIG. 14a shows that in MCF-7/ADR cells there are many punctate red-orange fluorescing compartments throughout the cytoplasm which is indicative of acidic organelles. FIG. 14b shows that in MCF-7 there is little red-orange fluorescence from acridine orange. This is diagnostic of few acidified organelles. Note also that the nucleus of MCF-7 cells takes up a greater amount of acridine orange than the nucleus of MCF-7/ADR cells. FIG. 14c shows that in MCF-10F cells, a non-transformed human breast epithelial cell line, there are also many punctate red-orange fluorescing compartments distributed throughout the cytoplasm indicative of acidic organelles. The scale bar is 5 μm.

FIGS. 15a-15b shows that specific loading of a pH probe into the cytosol of MCF-7 cells. Cells were scrape loaded with SNARF dextran as described in Example 4. The scale bar is 5 μm. In FIG. 15a 70 kD SNARF-dextran loaded into MCF-7 cells. The fluorescence was excluded from the nucleoplasm and was observed as diffuse cytoplasmic fluorescence. Due to being conjugated to a dextran, it cannot cross internal membranes. Thus it specifically reports the pH of the cytosol. In FIG. 15b MCF-7 cells were loaded with a 10 kD SNARF dextran. The probe is present both in the cytosol and nucleoplasm. The distribution of these probes in MCF-7/ADR cells is similar. Note that in a some cells the SNARF-dextran has a punctate distribution in some areas of the cytoplasm which may be due to an aberrant aggregation of SNARF-dextran. All pH measurements were taken from a region of cytoplasm where there was no aggregation of the probe.

FIGS. 16a-16d shows the effect of Monensin on acidification and Adriamycin distribution in MCF-7/ADR cells. Monensin disrupts the acidification of subcellular compartments in drug-resistant MCF-7/ADR cells and redistributes Adriamycin to the nucleus. The scale bar is 5 μm. In FIG. 16a MCF-7/ADR cells were incubated with acridine orange (6 μM) as described in FIG. 13. There is punctate red-orange fluorescence throughout the cytoplasm indicative of acidified organelles. In FIG. 16b Monensin (5 μM) was added to the solution bathing the cells in FIG. 16a. Thirty minutes after addition of monensin, there was a loss of the red-orange fluorescence observed within cytoplasmic compartments. This is indicative of a loss of acidification. In FIG. 16c Adriamycin was incubated with MCF-7/ADR cells as described in FIG. 10. Adriamycin is seen again accumulating in a perinuclear compartment that co-localizes with the lysosomes, recycling endosomes and TGN compartments (see FIG. 11). In FIG. 16d Monensin was added to the media bathing the cells in FIG. 16c. After thirty minutes, the perinuclear accumulation of Adriamycin has decreased and instead Adriamycin is found to accumulate within the nucleus. The distribution of Adriamycin now resembles that seen in drug-sensitive MCF-7 cells (FIG. 10b).

FIGS. 17a-17h shows the effect of inhibitors of the H+-ATPase on acidification and Adriamycin distribution in MCF-7/ADR cells. Inhibitors of the vacuolar proton ATPases disrupt the acidification of drug-resistant MCF-7/ADR cells and redistribute the Adriamycin to the nucleus as assayed by laser-scanning confocal microscopy. The scale bar in FIGS. 17c and 17d is 2 μM and in all other Figures it is 5 μM. In FIG. 17a MCF-7/ADR cells were labeled with Acridine orange as in FIG. 14. The punctate red-orange fluorescence in the cytoplasm is diagnostic for acidified organelles. The same cells as in FIG. 17a 30 minutes after addition of Bafilomycin A1 (500 nM) are shown in FIG. 17b. Note the disappearance of punctate red-orange cytoplasmic fluorescence indicative of reduced acidification. In FIG. 17c MCF-7/ADR cells were incubated with Adriamycin as in FIG. 10. The Adriamycin fluorescence is observed within punctate cytoplasmic organelles which co-localize with lysosomes (see FIGS. 11g-11i) and with a perinuclear compartment which co-localizes with the TGN (see FIGS. 11d-11f) and the recycling endosomes (see FIGS. 11a-11c). The same cells as in FIG. 17c 30 minutes after addition of Bafilomycin A1 (500 nM) are shown in FIG. 17d. The fluorescence of Adriamycin is substantially decreased in all cytoplasmic compartments and increased in the nucleoplasm. Acridine orange labeled MCF-7/ADR cells are shown in FIG. 17e. Note the red-orange cytoplasmic compartments which are diagnostic of acidified organelles. The same cells as in FIG. 17e 30 minutes after inclusion of concanomycin (100 nM) in the incubation media are shown in FIG. 17f. The punctate red-orange fluorescence from acridine orange accumulation is almost totally dissipated. FIG. 17g shows the fluorescence of Adriamycin in MCF-7/ADR cells.

The same cells as in FIG. 17g 30 minutes after inclusion of concanomycin (100 nM) in the incubation media are shown in FIG. 17h. There is a substantial decrease of Adriamycin fluorescence in punctate cytoplasmic organelles and pericentriolar compartment. In contrast, there is significant increase of Adriamycin fluorescence in the nucleus.

FIG. 18 shows the effect of Tamoxifen on Adriamycin sensitivity of MCF-7/ADR cells. The effects of Tamoxifen on the sensitivity of MCF-7/ADR cells to Adriamycin were studied by incubating cells with Tamoxifen and Adriamycin for 6 hours. Cell viability was measured three days later as described in materials and methods and plotted for cells treated in the absence (●) and presence of Tamoxifen (▪, 5 μM; 5, 10 μM) at varying concentrations of Adriamycin. Tamoxifen, at 5 μM, had little effect on cell viability in the absence of Adriamycin (left-most data point). However, Tamoxifen substantially increased the sensitivity of the cells to Adriamycin.

FIGS. 19A-19F shows the examination of the distribution of the Adriamycin in both drug-resistant MCF-7/adr cells and drug-sensitive MCF-7 cells. Cells were incubated with 5 μM Adriamycin for 30 minutes as described in materials and methods and examined under epi-fluorescence. A bright field image of MCF-7 cells is shown in FIG. 19A. Adriamycin fluorescence was observed for the same field cell under epi-fluorescence in FIG. 19B. The Adriamycin was seen diffuse throughout the cytoplasm with an increased fluorescence in the nucleoplasm of the cells. C. Superimposition of the bright field (green, from FIG. 19a) and Adriamycin (red, from FIG. 19b) images showed the accumulation of Adriamycin in the nucleii of the MCF-7 cells FIG. 19c. A bright field image of MCF-7/ADR cells is shown in FIG. 19d. Adriamycin fluorescence was observed for the same field of cells in FIG. 19e. The Adriamycin was seen both in a perinuclear compartment (the trans-Golgi network and recycling endosome compartments) and in discrete punctate cytoplasmic organelles (the lysosomes). Adriamycin was almost completely excluded from the nucleus. Superimposition of bright field (green, from FIG. 19d) and Adriamycin (red, from FIG. 19e) images showed a cap of Adriamycin fluorescence on one side of the nucleus in the MCF-7/ADR cells in FIG. 19f. The scale bar is 10 μm.

FIG. 20 shows the effects of other drugs which reverse MDR on Adriamycin distribution. The effect of Tamoxifen on the subcellular distribution of Adriamycin in drug-resistant MCF-7/ADR cells was examined with laser-scanning confocal microscopy. Adriamycin fluorescence in three MCF-7/ADR cells is shown in FIG. 20A. Adriamycin was restricted to cytoplasmic organelles. The perinuclear compartment co-localizes with the recycling endosomes and trans-Golgi network and the discrete punctate organelles co-localize with the lysosomes [Example 4]. Adriamycin fluorescence was excluded from the nucleus in the confocal image. This indicated that the slight nuclear fluorescence seen with epi-fluorescence (FIG. 19e) was the result of fluorescence above or below the nucleus. FIG. 20B shows the distribution of Adriamycin in the same MCF-7/ADR cells 30 minutes after the addition of Tamoxifen (10 μM). The Adriamycin concentration was substantially reduced in both the perinuclear compartment and the discrete punctate cytoplasmic organelles. Conversely, the concentration of Adriamycin in the nucleus was substantially increased. Cells were observed at 37° C. in a closed chamber under constant perfusion with 5% CO₂. The scale bar is 10 μm.

FIGS. 21A-21H shows acridine orange labeling of MCF-7 and MCF-7/ADR cells Acridine orange is a weak base which accumulates in acidic compartments. At higher concentrations there is a quenching of fluorescence in the green. Thus, acidic compartments, which accumulate acridine orange have a red-orange fluorescence. The acridine orange fluorescence from MCF-7 cells is shown in FIG. 21A. There was a relatively even green fluorescence with no red-orange fluorescence. This suggests that the cytoplasmic organelles were not acidified. FIGS. 21B-21D show acridine orange fluorescence in MCF-7/ADR cells. The punctate red-orange fluorescence in the cytoplasm indicated acidified organelles. Thirty minutes after addition of Tamoxifen (FIG. 21F with 5 μM Tamoxifen is the same field as in FIG. 21B), verapamil (FIG. 21G with 10 μM verapamil is the same field as FIG. 21C) or cyclosporin A (FIG. 21H with 10 μM cyclosporin is the same field as FIG. 21D) there was a loss of the red-orange cytoplasmic fluorescence in the MCF-7/ADR cells (this is the same field of cells as in 21B). This suggests that each of these treatments resulted in a loss of acidification in cytoplasmic organelles. The cells were incubated with 2 μg/ml acridine orange as described in materials and methods and examined in laser scanning confocal microscopy. The scale bar is 10 μm.

FIGS. 22A-22D shows acridine orange labeling of MCF-7 and MDA and CHO cells. FIG. 22A shows acridine orange in MDA-A1. A red-orange acridine orange fluorescence, indicative of acidic compartments, was observed both in disperse punctate cytoplasmic vesicles and in a peri-nuclear region of the drug-resistant human breast tumor line MDA-A1. This cell line does not express the estrogen-receptor. FIG. 22B shows acridine orange in MDA-A1 cells (21A) after Tamoxifen: The medium perfusing the chamber was changed to include 10 μM Tamoxifen and fifteen minutes later there was a substantial reduction in the red acridine orange fluorescence. This indicated a loss of acidification in the cytoplasmic organelles. FIG. 22C shows acridine orange in CHO cells. Discrete punctate cytoplasmic red acridine orange fluorescence was observed in the CHO cells throughout the cytoplasm, but with an enhanced concentration in the perinuclear region. FIG. 22D shows acridine orange in CHO cells after Tamoxifen: Thirty minutes after the inclusion of Tamoxifen there was a loss of red-orange fluorescence from the cytoplasm of the CHO cells. Cells were incubated with 2 μg/ml acridine orange as described in materials and methods and examined under laser-scanning confocal microscopy. The scale bar is 10 μM.

FIGS. 23A-23B shows the acidification of the lysosomes as probed with the weak base DAMP which accumulates in acidic organelles. Visualization of DAMP in the electron microscope has been used to quantify the pH in Golgi and lysosomes [Barasch et al., J. Cell Biol., 107:2137-2147 (1988); Barasch et al., Nature (London), 352:70-73 (1991)]. Electron micrograph of mouse anti-DNP and gold conjugated anti-mouse antibodies in MCF-7/ADR cells (FIG. 23A). The gold particles indicate accumulation of DAMP within cytoplasmic organelles (at arrow heads). The average density of gold particles was 7.02/μm² of lysosomal area. FIG. 23B shows cells that were incubated with Tamoxifen prior to DAMP had a substantial reduction of anti-DAMP labeling to 2.0 gold particles/μm² of lysosomal area (arrow heads). Cells were incubated with DAMP, then fixed and prepared for immuno-electron microscopy as described in the methods. The scale bar is 1 μM.

FIGS. 24A-24D show the effect of Tamoxifen on in vitro acidification of MCF-7/ADR organelles. FIG. 24A shows organelle acidification as assayed by incubating microsomes with acridine orange. The accumulation of acridine orange results in a quenching of emission in the green, which is observed as a decrease in total fluorescence emission. Microsomes were suspended in acridine orange and the fluorescence was observed. Five minutes after establishing baseline, 1 mM Tris-ATP was added to begin acidification (at 300 seconds). The presence of ATP shifted the total fluorescence. This was followed by a slow decrease of total fluorescence over the subsequent 1200 seconds (▪). To confirm that the decrease was the result acidification, 5 μM nigericin, a protonophore was added to dissipate pH gradients at t=1500 seconds. The acridine orange fluorescence returned the levels recorded immediately upon addition of ATP. When microsomes were preincubated with 8 μM Tamoxifen there was almost no acidification. This effect of blocking acidification was apparent at 1 μM (♦) and increased in a dose-dependent manner (▴ 2 μM; □ 4 μM). Pre-treatment of microsomes with 10 nM Bafilomycin A1 (Δ) also blocked acidification. Plots are normalized by the last five seconds after the pH gradient have been dissipated and in the presence of all added reagents. FIG. 24B show the plot f dose-response curve for effects of Tamoxifen on acidification. Acidification was assayed by quenching of acridine orange fluorescence, as in FIG. 24A. The percentage of quenched acridine fluorescence is calculated by dividing the initial slope of fluorescence quenching at various drug concentrations by that of the control. The effect of Tamoxifen was readily apparent at 1 μM, while at 8 μM, acidification was almost completely absent. FIG. 24C shows the effect of adding Tamoxifen during the acidification: The kinetics of the effect of Tamoxifen and Bafilomycin A1 on acidification were examined. Ten minutes after the addition of 1 mM Tris-ATP, 8 mM Tamoxifen or 100 nM Bafilomycin A1 were added. In the absence of Tamoxifen or Bafilomycin A1, the organelles continued to acidify, as assayed by quenching of acridine orange fluorescence. Addition of Bafilomycin A1 or Tamoxifen rapidly reversed acidification of the organelles. Ten minutes after addition of Tamoxifen or Bafilomycin A1, 5 μM nigericin was added. Plots were normalized at the point where Tamoxifen or Bafilomycin A1 were added. Addition of 8 μM Tamoxifen almost completely dissipated the pH gradient within 5 minutes. This was faster than 100 nM Bafilomycin A1, and much slower than 5 mM nigericin, added at time 1500 seconds. FIG. 24D shows the effect of Tamoxifen on acidification in recycling endosomes. Cells were incubated with FITC-transferrin, which is endocytosed selectively into the endosomal system. After lysing the cells a microsomal fraction was harvested. The microsomes were resuspended and the fluorescence emission at 520 nm from the FITC was monitored in response to excitation at 450 and 490 nm. Upon addition of ATP (t=1080 seconds) there was acidification of the lumen of the microsomes as assayed by decrease in the ratio of the 490:450 nm emission. Nigericin was added (t=2500 sec) to confirm that the fluorescent shift was the result of acidification.

FIGS. 25A-25B shows the kinetics of transport of BODIPY-transferrin to the surface. The kinetics of transport of the transferrin receptor from the recycling endosomes to the surface of the cells was quantified as described in the Example 5. After loading MCF-7/ADR cells with BODIPY-transferrin all unbound transferrin was washed from the cell. The cell-associated BODIPY-transferrin was followed in confocal microscopy. The rate of transport of transferrin to the surface was substantially slowed in cells treated with Tamoxifen (10 μM). After five minutes only 40% of the transferrin was still associated with the MCF-7/ADR cells (◯). In contrast, more than 90% remained with MCF-7/ADR cells that had been incubated with Tamoxifen ▾. Less than 10% of the transferrin remained with the control MCF-7/ADR cells after 25 minutes. More than 60% remained with the Tamoxifen treated cells. The rate of transferrin transport to the surface in MCF-7/ADR cells treated with Tamoxifen was similar to the rate of transferrin transport in drug-sensitive MCF-7 cells (●).

FIGS. 26 and FIG. 27 show the kinetics of transport of BODIPY-sphingomyelin to the surface. The kinetics of transport of the lipid sphingomyelin from the trans-Golgi network to the surface was quantified as described in the Example 5. Cells were incubated with BODIPY-ceramide which is converted by the cell into BODIPY-sphingomyelin. The sphingomyelin transiently accumulates in the Golgi. Fluorescence of the BODIPY sphingomyelin was examined in the MCF-7/ADR cells after removal of BODIPY-ceramide from the cells and medium. Confocal images of the BODIPY fluorescence are shown at various time points in FIG. 26 and the total cell-associated BODIPY fluorescence is quantified in FIG. 27. The BODIPY-sphingomyelin accumulates in a perinuclear position (yellow fluorescence adjacent to the nucleus) in a compartment which has been identified as the trans-Golgi network [Pagano et al., J. Cell Biol., 113:1267-1279 (1991)]. After removal of the BODIPY-ceramide, the BODIPY fluorescence decreases in the MCF-7/ADR cells (left column of FIG. 26 and ◯ in FIG. 27). After two hours the cell-associated sphingomyelin is reduced to almost 20%. In the presence of Tamoxifen (10 μM) the BODIPY-fluorescence decreases more slowly from the MCF-7/ADR cells (middle column of FIG. 26 and in FIG. 27). In the MCF-7 cells (right column of FIG. 26 and in FIG. 27) the rate of transport of the BODIPY-sphingomyelin to the surface is similar to that of the MCF-7/ADR cells with Tamoxifen. The scale bar is 10 μM.

### DETAILED DESCRIPTION OF THE INVENTION

In accordance with the present invention, methods and associated assays are described for the monitoring of the likelihood or onset of multidrug resistance in mammals, and the identification and monitoring of agents serving to alleviate the effects of multidrug resistance particularly in relation to the treatment of cancerous tumor cells.

Although not constrained to any particular model or mechanism the present invention is consistent with the proposition that the protonation, sequestration and secretion (PSS model) of chemotherapeutics within the efflux pathway should not be functional in the acidification-deficient drug-sensitive cells. Thus, the present invention is consistent with the premise that an alkaline pH shift observed in intracellular vesicular compartments of the cell during multidrug resistance and/or a defect in the vesicular transport mechanism are sufficient to account for the observed decreases of cellular accumulation of chemotherapeutics and the observed decreased sensitivity of drug-resistant cells to chemotherapeutics.

As used herein a “secretory compartment” is an intracellular vesicular compartment e.g., an organelle, that is involved in the export of chemical substances including biomolecules such as lipids and proteins from the cell. Examples of secretory compartments include the perinuclear recycling compartment (PRC), the recycling endosomes, the secretory vesicles, and the trans-Golgi network (TGN).

As used herein, a “marker used for determining whether there is a defect in the vesicular transport mechanism” is an indicator whose absence or presence can be determined and/or quantified and used to ascertain the effectiveness of the vesicular transport of an intracellular compartment. For example, the endocytic system can be used to take in any marker including a sugar, e.g., dextran, or a protein, e.g., ferritin, which can be endocytosed. Markers of the present invention include compounds that can be monitored by an intrinsic property such as fluorescent labeled proteins, e.g. a labeled transferrin, and labeled lipids e.g. labeled sphingomyelin. Such labels (exemplified below) can be adsorbed or bound to a particular biomolecule of choice, including through a covalent bond (e.g. a chimeric protein comprising transferrin and green fluorescent protein). The markers of the present invention can also have an intrinsic biological activity which can be determined or be labeled with an enzyme that has a biological activity that can be determined (e.g. a chimeric protein comprising transferrin and luciferase).

As used herein, “measuring the transport” of a marker from an intracellular vesicular compartment can be performed by any means that ascertains the effectiveness of the intracellular vesicular compartment to transport the marker to the cell surface or cell exterior. Such means include determining the absence or presence of the marker in the intracellular vesicular compartment and/or on the cell surface or cell exterior; quantifying the amount of marker remaining in the intracellular vesicular compartment and/or on the cell surface or cell exterior; and measuring the rate of transport of a marker from the intracellular vesicular compartment and/or to the cell surface or cell exterior. These measurements are made with respect to a drug-sensitive tumor cell that has a defect in the vesicular transport mechanism and in which the cell is suspected of developing or already having become multidrug resistant due to an increase in the effectiveness of the intracellular vesicular compartment to transport drugs to the cell surface or cell exterior. Such a multidrug resistant cell is identified when the relative measure of transport of a marker increases, e.g. an increase in the amount of marker transported to the cell surface or cell exterior, and/or the rate of transport of the marker from the intracellular vesicular compartment to the cell surface or exterior increases.

Similarly, as used herein, a “measure of the pH” of an intracellular vesicular compartment can be any determination that can be correlated to the pH of the intracellular vesicular compartment. Such means include measuring the pH directly, or indirectly as further exemplified below. The pH of an intracellular vesicular compartment can be directly determined with an electrode or a calibratable pH indicator or pH sensitive probe. Alternatively the minimum (or maximum) pH can be determined, e.g. using a pH indicator or pH sensitive probe that has an altered property (such as fluorescence or color) below (or above) a particular pH. Such measurements can be made with respect to a drug-sensitive tumor cell that has a defect in the acidification of an intracellular vesicular compartment and in which the cell is suspected of developing or having become multidrug resistant due to a decrease in the pH of the intracellular vesicular compartment. A multidrug resistant cell can be identified when a measure of the pH of an intracellular vesicular compartment indicates a decrease in pH of the intracellular vesicular compartment.

Examples of direct pH sensitive probes include acridine orange and Lysosensor Blue DND-167, exemplified below, which can be used for determining the pH of the intracellular vesicular compartments. pH sensitive probes can also be targeted to specified intracellular vesicular compartments. For example, a pH-sensitive probe can be targeted via specific receptors to the endosomes, e.g., using the transferrin receptor, or to the Golgi, using particular toxins such as verotoxin.

A defect or disruption in the acidification of an intracellular vesicular compartment of a cell can also be determined through an indirect measure of pH such as assaying for the consequences of having a defect in the acidification of an intracellular vesicular compartment. For example, a defect and/or disruption of the acidification of an intracellular vesicular compartment can be determined by detecting a change in the glycosylation of lipids or proteins on the surface of the cell. One means of making this determination is through the use of a lectin. Preferably the lectin is labeled. In one such embodiment the defect and/or disruption of acidification of an intracellular vesicular compartment can be measured by selectively assaying for the presence of sialic acids attached to lipids or proteins on the surface of the cell either directly (e.g., a lectin such as the elderberry lectin, sambucus nigra) or indirectly (e.g., by cell adhesion studies explained below). A decrease in the presence of sialic acids is indicative of a defect or disruption of the acidification of an intracellular vesicular compartment. In this case, the pH optimum for the α2-6 sialyl transferase in mammary tissue is pH 5.9. The pH is usually 5.9 to 6.0 in the relevant intracellular vesicular compartment. Thus, when the intracellular vesicular compartment pH shifts from the pH optimum, there is a decrease in the addition of sialic acids. Again a drug-sensitive tumor cell that has a defect or disruption of the acidification of the intracellular vesicular compartment can be compared with the tumor cell suspected to be developing or has already become multi-drug resistant which no longer has the defect.

In addition, a defect or disruption of the acidification of an intracellular vesicular compartment can be measured indirectly by a change in the secretion of lysosomal enzymes from the cell. When the amount of lysosomal enzymes secreted by a tumor cell is increased, it is indicative of a defect or disruption of the acidification of an intracellular vesicular compartment. A multidrug resistant cell in which the defect or disruption of the acidification is alleviated thus shows a decrease in the amount of lysosomal enzymes secreted.

The PSS mechanism for drug resistance is based on the following five experimentally determined observations and conclusions: 1) Chemotherapeutics accumulate in the acidic organelles of drug-resistant cells and diffuse through the cytosol of drug-sensitive cells. 2) The intracellular organelles of drug-sensitive cells either more alkaline than the wild type cells and/or have a defect in their transport system to the cell surface. 3) Agents that disrupt organelle acidification (protonophores such as monensin, nigericin, or blockers of the H+-ATPase) reverse the drug resistance of MDR tumor cells. 4) Agents that disrupt transport of organelles to the plasma membrane e.g., inhibitors of ceramide synthase) reverse the drug resistance of MDR tumor cells. 5) Agents that reverse the drug-resistance of MDR tumor cells disrupt either organelle acidification or transport through the exocytotic pathway.

As reported herein a widely administered chemotherapeutic drug Adriamycin accumulates in punctate acidic compartments (lysosomes, TGN and recycling endosomes) of the drug-resistant MCF-7/ADR cells, whereas, in the drug-sensitive MCF-7 cells, it is broadly distributed throughout the nucleoplasm and cytoplasm. The pH was quantified in the cytosol, nucleus, recycling endosomes, and lysosomes of both drug-sensitive and drug-resistant cells. The pH profile of the MCF-7/ADR cells was similar to that previously observed for non-transformed cells [Tycko and Maxfield, Cell, 28:643-651 (1982); Yamashiro and Maxfield, J. Cell Biol., 105:2723-2733 (1987); Augenbraun et al., Eur. J. Cell Biol., 61:34-43 (1993)]: the cytosolic pH was neutral and the lysosomes and endosomes were acidified. In contrast, the organelles of drug-sensitive MCF-7 cells had substantially reduced transmembrane pH gradients. Agents that disrupted acidification of organelles (e.g. the H+-ATPase blockers Bafilomycin A1 and concanamycin or the protonophores monensin and nigericin) were found to dissipate the chemotherapeutic drugs from the acidified organelles and reverse drug-resistance.

The diagnostic application of the invention is partially based on the observation that shifting intracellular pH is sufficient to either decrease the concentration of anti-cancer agents in drug-sensitive cells or increase their concentration in drug-resistant cells. Therefore it is one goal of the present invention to identify chemicals that de-acidify the intracellular compartments of the MDR cells and re-sensitize them to anti-tumor drugs. The acidifying agents could then be coupled with anti-tumor drugs during chemotherapy.

One approach derived from the experiments underlying the invention is to load drug-resistant cell lines and non-transformed cells with a pH indicator such as SNARF1. The pH of both cell types are then measured in response to a wide range of dosages of drugs that are used to modify cellular pH. There are two different strategies that will be used to select drugs for testing. Based on the observations on the mechanisms by which MDR proteins affect cellular pH drugs can be tested that interfere with the ability of MDR proteins to affect cellular pH. However, given that the pH regulatory mechanisms of tumor cells are compromised relative to the regulatory mechanisms of normal cells, the large number of drugs that have been developed by the pharmacological industry to affect pH (primarily for the purpose of combating ulcers) can also be tested.

The strategy for identifying compounds that can reverse MDR depends upon finding drugs that have a greater effect on MDR cells is provided. These compounds may be given in conjunction with normal chemotherapeutic agents to kill tumors.

As disclosed herein, acidification of intracellular compartments is essential for correct sorting and transport of proteins during endocytosis and secretion. A human breast cancer line (MCF-7) that is aberrant in acidification of intracellular vesicular compartments is exemplified below. This defect is correlated with a disruption in the organization and function of the trans-Golgi network (TGN) and the pericentriolar recycling compartment. In marked contrast, human breast cancer cells (MCF-7adr) that are resistant to the most widely employed chemotherapeutic drug, adriamycin, appear normal in both acidification of intracellular vesicular compartments and in the organization of the recycling and secretory compartments. Treatment of drug resistant MCF-7adr cells with nigericin and monensin, ionophores which have been shown to disrupt vesicular acidification [Tartakoff, Cell, 32:1026-1028 (1983)], results in sensitization of these cells to adriamycin. Drug sensitivity therefore results from an acidification defect within vesicles of the recycling and secretory pathways. The functional consequence of this defect is the diminished capacity of cells to remove drugs from the cytosol through vesicle mediated protonation, sequestration, and secretion of cytotoxic drugs.

As further disclosed herein, defective acidification within exocytotic compartments in adriamycin-sensitive MCF-7 human breast cancer cells is correlated with fragmentation of the trans-Golgi network (TGN) and disruption of the pericentriolar recycling compartment (PRC). These defects are repaired in drug resistant cells (MCF-7adr). Brief treatments of drug resistant MCF-7adr cells with Tamoxifen reverts their phenotype to resemble the drug-sensitive cells including: (a) disruption of the structure of the TGN and PRC, (b) loss of acidification in exocytotic compartments, (c) redistribution of adriamycin from exocytotic compartments to the nucleoplasm, and (d) increased sensitivity to chemotherapy. Reversal of drug-resistance therefore results from Tamoxifen mediated inhibition of acidification within exocytotic compartments.

Indeed, multidrug resistance (MDR) is a significant clinical problem in the chemotherapeutic treatment of cancer. Chemotherapeutics distribute diffusely through the cytoplasm and nucleoplasm of drug-sensitive cells but are excluded from the nucleus and, instead, concentrated in cytoplasmic organelles of drug-resistant cells. Many chemotherapeutics such as anthracyclines and vinca alkaloids are weak bases which should concentrate in the lumen of acidic cytoplasmic organelles. The potential role of pH in drug sensitivity and resistance was examined herein and a pH profile is quantified for identified subcellular compartments of drug-sensitive and -resistant human breast cancer cells. The results demonstrate that: a) the chemotherapeutic drug Adriamycin concentrates in acidified organelles of drug-resistant MCF-7/ADR cells, whereas it does not concentrate in organelles of drug-sensitive MCF-7 cells; b) the lysosomes and recycling endosomes are not acidified in drug-sensitive MCF-7 cells, c) the cytosol of drug-sensitive MCF-7 cells is 0.4 pH units more acidic than the cytosol of drug-resistant MCF-7/ADR cells; d) disrupting the acidification of the organelles of MCF-7/ADR cells with monensin, Bafilomycin A1 or Concanomycin A is sufficient to change the Adriamycin distribution to that found in drug-sensitive MCF-7 cells rendering the cell vulnerable once again to chemotherapy. These results indicate that acidification of organelles is causally related to drug resistance and is consistent with the hypothesis that sequestration of drugs in acidic organelles and subsequent extrusion from the cell through the secretory pathways contributes to chemotherapeutic resistance.

In multidrug-resistant (MDR) human breast cancer cells, the chemotherapeutic drug Adriamycin accumulates within their acidic organelles and is absent from the nucleoplasm. In contrast, drug-sensitive cells lack acidic organelles and Adriamycin is dispersed throughout the cytoplasm and nucleoplasm. The sensitivity of non-MDR tumor cells to chemotherapeutics is a consequence of their inability to protonate and sequester drugs in their cytoplasmic organelles. Conversely, the reduced sensitivity of MDR cells is the consequence of the protonation and sequestration of drugs within acidic organelles, followed by secretion from the cell. Blocking organelle acidification with monensin, nigercin, Bafilomycin A1 or concanomycin A reverses a cell's MDR [Schindler et al., Biochemistry, 35:2811-2817 (1996); Example 4, below]. As disclosed herein agents that reverse MDR (verapamil, Tamoxifen, cyclosporin A) also reverse organelle acidification, reverse accumulation of chemotherapeutics in the organelles and slow transport through the secretory pathway at the concentrations at which they reverse MDR. The estrogen-receptor antagonist Tamoxifen directly inhibits acidification within isolated secretory organelles, independent of the expression of estrogen receptors. These observations provide evidence for a causal relationship between disruption of acidification within exocytotic compartments, and the reversal of Adriamycin resistance.

In a further aspect of the invention, therapeutic methods and corresponding formulations are contemplated. For example, the agents identified by the assays of the present invention may be administered in conjunction with conventional chemotherapeutic agents, either individually or in a cocktail, or alternately in complex of the agent and the chemotherapeutic. The complex may be prepared in pharmaceutical compositions that in turn, may be administered by those routes conventional for drugs of this type. For example the compositions may be administered by oral or parenteral means, such as intravenous.

The data presented in the following examples support the underlying concept of the invention that an additional mechanism of MDR by which changes in intracellular pH alter the transmembrane partitioning or intracellular sequestration of drugs. Cytosolic pH affects protonation of these drugs (typically weak bases, pKa's between 7.4-8.2 [Di Marco et al., Chem. Biol. Interact., 19:291-302 (1977); Owellen et al., Biochem. Pharm., 26:1213-1219 (1977); Skovsgaard, Biochem. Pharm., 26:215-222 (1977)]), affinity of intracellular sites for drug binding, and/or secretion from organelles which accumulate the drugs. The pH of tumor cells is considerably more acidic than that of normal [Warburg, Science, 123:309-314 (1956)] or MDR [Thiebaut et al., J. Histochem. Cytochem., 38:685-690 (1979)] cells. Drugs which partition across the membrane would be protonated and ionically trapped in the cytosol in their biologically active form (the charged form of these drugs binds to their targets such as DNA [Zunino et al., Chem. Biol. Interact., 24:217-225 (1979); Zunino et al., Biochim. Biophys. Acta, 476:38-46 (1977); Zunino et al., Biochim. Biophys. Acta, 277:489-498 (1972); Di Marco et al., J. Cell Biol., 27:545-550 (1965); Calendi et al., Biochim. Biophys. Acta, 103:25-49 (1965)], RNA [Calendi et al., Biochim. Biophys. Acta, 103:25-49 (1965); Doskocil & Fric, FEBS Letters, 37:55-58 (1973)] and tubulin [Weisenberg & Timasheff, Biochemistry, 9:4110-4116 (1970); Na & Timasheff, Archives of Biochemistry and Biophysics, 182:147-154 (1977)]). The acidic pH of tumor cells would increase their sensitivity to the drugs. The P-glycoprotein, as well as other proteins that are correlated with MDR, could affect the activity of chemotherapeutic agents by modification of pH homeostasis.

Gradients of pH have been used to trap these drugs in liposomes and red blood cells. To test whether the pH gradients observed in tumors and MDR significantly change intracellular drug concentration, the kinetics of accumulation in drug-sensitive cells were examined where the pH was manipulated experimentally to mimic that observed in resistant cells.

For fluorescence spectroscopy the pH indicator is preferably sensitive only to the extremely acidic environments, e.g., as that found in the lysosomes. Examples of such indicators are Lysosensor Blue DND-167 or acridine orange. Acridine orange emission in the red is the assay for formation of a pH gradient across intracellular membranes. Lysosensor Blue DND-167 only emits fluorescence below pH 5.8 and its emission is diagnostic of formation of a pH gradient across the intracellular membranes.

Appropriate pH indicators for fluorescence microscopy include but in no way is limited to the use of any vital pH-indicator, acridine orange, Lysosensor Blue DND-167, SNAFL, SNARE, BCECF, FITC, and DAMP. Appropriate pH indicators for confocal microscopy include but in no way is limited to any vital pH-indicator, acridine orange, Lysosensor Blue DND-167, SNAFL, SNARE, and BCECF, FITC, and DAMP.

Drug Screening

A potential drug can be obtained by a number of means including from a commercially available chemical library such as is available from most large chemical companies including Merck, Glaxo Welcome, Bristol Meyers. Squib, Monsanto/Searle, Eli Lilly, Novartis, and Pharmacia Upjohn. Potential drugs can also be synthesized de novo or obtained from phage libraries.

Phage libraries have been constructed which when infected into host E. coli produce random peptide sequences of approximately 10 to 15 amino acids [Parmley and Smith, Gene 73:305-318 (1988), Scott and Smith, Science 249:386-249 (1990)]. Once a phage encoding a peptide that can act as a potential drug has been purified, the sequence of the peptide contained within the phage can be determined by standard DNA sequencing techniques. Once the DNA sequence is known, synthetic peptides can be generated which are encoded by these sequences.

These peptides can be tested, for example, for their ability to e.g., (1) decrease vesicular transport in a multidrug resistant tumor cell or (2) to interfere with the acidification of an intracellular vesicular compartment of a multidrug resistant tumor cell.

The effective peptide(s) can be synthesized in large quantities for use in in vivo models and eventually in humans to overcome multidrug resistance. It should be emphasized that synthetic peptide production is relatively non-labor intensive, easily manufactured, quality controlled and thus, large quantities of the desired product can be produced quite cheaply. Similar combinations of mass produced synthetic peptides have been used with great success [Patarroyo, Vaccine, 10:175-178 (1990)].

The drug screening methods of the present invention can use a variety of different multidrug resistant tumor cells or tumor cell lines including those readily available from the American Type Culture Collection such as uterine sarcoma cells, leukemia cells, colorectal carcinoma cells, mammary cells (as exemplified below), and neuroblastoma drug-resistant cells.

The present invention also includes kits for screening a potential drug for the treatment of multidrug resistance (MDR). One such kit includes a mammalian multidrug resistant tumor cell and a labeled marker that can be used to measure the transport to the cell surface from the intracellular compartment of the cell. In a preferred embodiment of this type, a protocol is included. Any of the multidrug resistant cells described above can be provided. Similarly, any of the markers of the present invention can be included. In a particular embodiment, labeled transferrin is provided. In another embodiment, labeled ceramide is provided.

Labels

Suitable labels include enzymes, pH-sensitive fluorophores as described in the Examples below, as well as other fluorophores such as (e.g. fluorescein isothiocyanate (FITC), phycoerythrin (PE), Texas red (TR), rhodamine, free or chelated lanthanide series salts, especially Eu³⁺, to name a few fluorophores), chromophores, radioisotopes, chelating agents, dyes, colloidal gold, latex particles, ligands (e.g., biotin), and chemiluminescent agents. When a control marker is employed, the same or different labels may be used for the test and control marker gene.

In the instance where a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re are used, known currently available counting procedures may be utilized. In the instance where the label is an enzyme, detection may be accomplished by any of the presently utilized colorimetric, spectrophotometric, fluorospectrophotometric, amperometric or gasometric techniques known in the art.

Direct labels are one example of labels which can be used according to the present invention. A direct label has been defined as an entity, which in its natural state, is readily visible, either to the naked eye, or with the aid of an optical filter and/or applied stimulation, e.g. U.V. light to promote fluorescence. Among examples of colored labels, which can be used according to the present invention, include metallic sol particles, for example, gold sol particles such as those described by Leuvering (U.S. Pat. No. 4,313,734); dye sole particles such as described by Gribnau et al. (U.S. Pat. No. 4,373,932 and May et al. (WO 88/08534); dyed latex such as described by May, supra, Snyder (EP-A 0 280 559 and 0 281 327); or dyes encapsulated in liposomes as described by Campbell et al. (U.S. Pat. No. 4,703,017). Other direct labels include a radionucleotide, a fluorescent moiety including a green fluorescent protein and its derivatives as described in U.S. Pat. No. 5,625,048 filed Apr. 29, 1997 and WO 97/26333, published Jul. 24, 1997 each of which are hereby incorporated by reference herein in their entireties, or a luminescent moiety. In addition to these direct labeling devices, indirect labels comprising enzymes can also be used according to the present invention. Various types of enzyme linked immunoassays are well known in the art, for example, alkaline phosphatase and horseradish peroxidase, lysozyme, glucose-6-phosphate dehydrogenase, lactate dehydrogenase, urease, these and others have been discussed in detail by Eva Engvall in Enzyme Immunoassay ELISA and EMIT in Methods in Enzymology, 70:419439 (1980) and in U.S. Pat. No. 4,857,453. Suitable enzymes include, but are not limited to, alkaline phosphatase, β-galactosidase, luciferase, horseradish peroxidase. Other labels for use in the invention include magnetic beads or magnetic resonance imaging labels.

Labeling and pH Measurements

Cells and cellular vesicular compartments can be labeled with any number of pH-sensitive compounds for the practice of the present invention. Below are examples of such compounds and methodologies which can be used but are no way meant to limit the compounds or methodologies that can be employed by the present invention.

Adriamycin Labeling: Adriamycin is a small heterocyclic amine (molecular wt. 580 Dalton) with a pK of 8.3 that can diffuse across membranes in the uncharged form. Adriamycin can be excited between 350 nm and 550 nm and emits between 400 nm and 700 nm. As exemplified below cells can be incubated with Adriamycin (10 μM) for 30 minutes at 37° C. and then visualized with a confocal microscope using 488 nm line of the argon laser. For epi-fluorescence, as exemplified below, the cells can be excited with 450-490 nm filter and emission monitored with a 510 nm longpass filter.

Acridine orange labeling: As exemplified below cells can be incubated with acridine orange (6 μM) for 15 minutes and then visualized on the confocal microscope with the 488 nm line of the argon laser. The green and red emissions can be collected using two photo multiplier tubes. Emission light was first passed through a 560 nm short-pass dichroic mirror. Green emission was next passed through a 530 nm/30 nm bandpass filter and collected by the on-axis photo multiplier tube. Red emission was passed through a 610 nm longpass filter and collected by the 90° side photo multiplier tube. In an alternative procedure cells in the presence of acridine orange were examined utilizing an excitation at 488 nm and dual emission confocal images simultaneously recorded using both a 530-30 band pass barrier filter (green fluorescence) and a 605 nm long pass barrier filter (red fluorescence). Optical sections of the fluorescent samples can be recorded at 0.5 micron intervals with a 60×oil immersion objective as exemplified below.

Lysosome labeling: Cells can be incubated with Lysosensor Blue DND 167 [Haugland, in Molecular Probes, Hand Book of Fluorescent Probes and Research Chemicals, 6th ed., Eugene, Oreg., p.278 (1996)] (2 μM, 1 mM stock in water) and then visualized on the confocal microscope using the 353 nm line of the argon laser.

TGN labeling with NBD-ceramide: Cells growing on Labtek coverslip chambers can be incubated in DMEM/20 mM HEPES pH 7.3 containing of NBD-Ceramide (5 μM) at 4° C. for 10 minutes [Pagano et al., J. Cell Biol., 113:1267-1279 (1991)]. They can then be washed and incubated at 37° C. for 30 minutes and then placed on the confocal microscope for observation using the 488 nm line of the Argon laser as exemplified below.

Bodipy-transferrin labeling of the recycling endosome compartment: BODIPY-transferrin can be used to label the recycling endosome compartment for structural studies. Transferrin is endocytosed by specific transferrin receptors on the surface of the cell. After endocytosis the transferrin is transported through the endosomes and then recycled back to the surface. The transferrin receptor is not transported to the lysosomes, so probes that are conjugated to transferrin can be used to selectively monitor the recycling endocytic compartments [Fuller and Simons, J. Cell Biol., 103:1767-1779 (1986); Ghosh and Maxfield, J. Cell Biol., 128:549-561 (1995)]. The endocytic pathway is known to undergo acidification [Schmid et al., J. Cell Biol., 108:1291-1300 (1989)]. Thus, the fluorophore BODIPY can be used as a probe on transferrin since its fluorescence is not very sensitive to pH.

pH measurements: The pH sensitive fluorophores, FITC and SNARF, can be used to measure the pH within endosomes and the cytosol, respectively. Lysosensor Blue DND-167 is a fluorophore that can be used as an independent probe specifically for calibration of the pH within the lumenal compartment of lysosomes. Both FITC and SNARF are ratio metric dyes. The emission intensity of FITC at 530 nm increases with increasing pH with excitation at 490 nm. However, it is unaffected by pH when the fluorophore is excited at 450 nm. Therefore, by taking the ratio of the emission intensities at the two excitation wavelengths, one can obtain a pH value independent of FITC concentration in a particular compartment. FITC is most useful for measurement of pH values from 5.0 to 7.0. SNARF, when excited at 514 nm, emits at two wavelengths: 570 nm and 630 nm. The protonated fluorophore emits at 570 nm and the neutral fluorophore emits at 630 nm. Again, the ratio of the two emissions corresponds to a pH value that is independent of the concentration of the dye in that compartment. SNARF can be reliably calibrated over the pH range of 6.2 to 9.0. The fluorescence of Lysosensor Blue DND-167 is dependent on pH. Lysosensor Blue has a functional group that, when deprotonated, leads to a loss of fluorescence of the molecule. The pK of this group is 5.1 Therefore at pH<5.1, a greater percent of the dye will be protonated and will be fluorescent. There is little fluorescence above pH 5.8. At the end of an experiment, to convert the ratios to pH values the fluorescence emission of each dye can be calibrated with solutions of known pH as exemplified below.

Organelle-specific pH measurements: The pH can be measured in selective cellular compartments by targeting ratio metric pH probes to specific organelles. For example, using the confocal microscope, the pH probe SNARF was excited at 514 nm and its emission was recorded simultaneously on two orthogonal PMT's using a 610 nm dichroic a 570/30 nm bandpass filter and a 630 nm longpass filter. Using a epi-fluorescence microscope with a intensified CCD camera, the pH probe FITC can be excited alternately at 450 nm and 490 nm and emission recorded with a 520/10 bandpass filter.

pH in the recycling endosomes: The transferrin receptor has been used as a selective probe for the recycling endosome pathway [Fuller and Simons, J. Cell Biol., 103:1767-1779 (1986); Roff et al., J. Cell Biol., 103:2283-2297 (1986); Sipe and Murphy, Proc. Natl. Acad. Sci. USA, 84:7119-7123 (1987); Stoorvogel et al., J. Cell Biol., 106:1821-1829 (1988); Dunn et al., J. Cell Biol., 109:3303-3314 (1989); Mayor et al., J. Cell Biol., 121:1257-1269 (1993); McGraw et al., J. Cell Biol., 155:579-594 (1993)]. After endocytosis, the transferrin is transported through the endosomes and then recycled back to the surface without passage through the lysosomes. Thus, the pH of the recycling endosomes can be selectively monitored by conjugating a pH probe, such as FITC or SNARF, to transferrin [Dunn et al., J. Cell Biol., 109:3303-3314 (1989)] Examples 4 and 5, below].

Recycling compartment pH measurement: The probe FITC bound to transferrin can be used to selectively probe the pH of the endocytic compartment. FITC [Schmid et al., J. Cell Biol., 108:1291-1300 (1989); Ghosh and Maxfield, J. Cell Biol., 128:549-561 (1995)]. The cells can be loaded with FITC-transferrin using the same protocol used to label the endocytic compartment with BODIPY-transferrin. The pH can be calibrated from the FITC fluorescence as described herein.].

pH in the lysosomes: The pH in the lysosomes can be assayed both with light and electron microscopy. Light microscopy: Cells can be incubated with FITC-dextran 10 kD (5 mg/ml) (DME/HERES) for 30 minutes, washed 4 times with DME/HERES, incubated for an additional 90 minutes to chase out the endosomes and visualized on a Nikon Diaphot equipped with FITC excitation filters (as exemplified below) [Yamashiro and Maxfield, J. Cell Biol., 105:2723-2733 (1987)]. The pH can then be calibrated. Alternatively cells can be incubated with Lysosensor Blue as described herein. Electron microscopy: The cells can be incubated with the weak base DAMP, fixed, probed with an mouse antibody to DNP (cross-reacts with DAMP) and visualized with gold-conjugated anti-mouse antibodies. This can be used to quantify the pH in different cellular organelles [Barasch et al., J. Cell Biol., 107:2137-2147 (1988); Barasch et al., Nature (London), 352:70-73 (1991)].

pH of the Cytoplasm and Nucleoplasm: The pH within the cytoplasm and nucleoplasm can be selectively probed for example by loading these compartments with the ratio metric pH probe SNARF conjugated to dextrans using a procedure referred to as “scrape loading” [McNeil et al., J. Cell Biol., 98:1556-1564 (1984)]. As exemplified below, the cells can be plated on polystyrene plates at 50% confluency 24-36 hours before loading with dextrans. The medium is then aspirated off the dishes, and the cells are covered with 50 μL of the SNARF dextran at 10 mg/ml concentration. The cells are then scraped off the polystyrene with a rubber scraper and placed in pre-chilled tubes containing 1 ml of media without serum. The cells can be harvested by spinning at a force of 100 g for 5 minutes as exemplified below. The cytosolic pH can be selectively probed by loading the cytosol with a 70 kD SNARF-conjugated dextran. This dextran is too large to enter into organelles or the nucleus. The nucleoplasmic pH can be probed by loading the cytosol with SNARF conjugated to a 10 kD dextran. This is too large to cross cellular membranes, but can enter the nucleoplasm by diffusion across the nuclear pores. Confocal fluorescence microscopy can be used to prepare optical sections through the cell as exemplified below. The fluorescence intensity of the nucleoplasm and cytoplasm could then be quantified.

Acidification of Cellular Microsomes: The acidification of cellular microsomes can be assayed spectrophotometrically. Two different approaches are exemplified below which can be used for assaying acidification are (a) Acidification of the total microsomal preparation using quenching of acridine orange and (b) Acidification of the recycling endosomes by monitoring the fluorescence from a microsomal preparation from cells that had previously endocytosed FITC-transferrin.

Intracellular Vesicular Transport Assays

Transport Assays: Transport of transferrin from recycling endosomes to cell surface Transferrin can be used to selectively label the recycling endosomes of cells [Fuller and Simons, J. Cell Biol., 103:1767-1779 (1986); Roff et al., J. Cell Biol., 103:2283-2297 (1986); Sipe and Murphy, Proc. Natl. Acad. Sci. USA, 91:3497-3504 (1987); Stoorvogel et al., J. Cell Biol., 106:1821-1829 (1988); Dunn et al., J. Cell Biol., 109:3303-3314 (1989); Mayor et al., J. Cell Biol., 121:1257-1269 (1993); McGraw et al., J. Cell Biol., 155:579-594 (1993)]. The endocytic pathway is known to undergo acidification. Thus, the fluorophore BODIPY can be used as a probe on transferrin since its fluorescence is photostable and insensitive to pH. Transport of transferrin can be assayed as previously described [Ghosh and Maxfield, J. Cell Biol., 128:549-561 (1995)] as exemplified below.

Transport of sphingomyelin from TGN to cell surface: BODIPY-ceramide labels endomembranes and its metabolic product, BODIPY-sphingomyelin, accumulates within the Golgi compartments [Pagano et al., J. Cell Biol., 113:1267-1279 (1991)]. When accumulated at high concentrations, BODIPY-sphingomyelin undergoes a green to red shift in fluorescence emission. Excitation can be performed at 488 nm and dual emission imagescan be prepared utilizing the filter set described for acridine orange and a 100×oil immersion objective. Efflux studies with BODIPY-ceramide are exemplified below.

Pharmaceuticals

The present invention also provides methods for treating multidrug resistance in a tumor cell. One such embodiment consists of administering to the tumor cell (or animal) an agent in an amount effective for disrupting the acidification of an intracellular vesicular compartment of the tumor cell and thereby alleviating the multidrug resistance in the tumor cell (or animal). Another such embodiment consists of administering to the tumor cell (or animal) an agent in an amount effective for disrupting the vesicular transport mechanism of an intracellular vesicular compartment of the tumor cell and thereby alleviating the multidrug resistance in the tumor cell. According to the invention, the agent can be part of a therapeutic composition which could also contain a chemotherapeutic agent. The therapeutic composition may be introduced parenterally, e.g., via intravenous injection, and also including, but is not limited to, intra-arteriole, intramuscular, intradermal, subcutaneous, intraperitoneal, intraventricular, and intracranial administration. transmucosally, e.g., orally, nasally, or rectally, or transdermally.

In another embodiment, the therapeutic composition can be delivered in a vesicle, in particular a liposome [see Langer, Science 249:1527-1533 (1990); Treat et al., in Liposomes in the Therapy of Infectious Disease and Cancer, Lopez-Berestein and Fidler (eds.), Liss: New York, pp. 353-365 (1989); Lopez-Berestein, ibid., pp. 317-327; see generally ibid.]. To reduce its systemic side effects, this may be a preferred method for introducing the agent.

In yet another embodiment, the therapeutic compound can be delivered in a controlled release system. For example, the agent may be administered using intravenous infusion, an implantable osmotic pump, a transdermal patch, liposomes, or other modes of administration. In one embodiment, a pump may be used [see Langer, supra; Sefton, CRC Crit. Ref Biomed. Eng. 14:201 (1987); Buchwald et al., Surgery 88:507 (1980); Saudek et al., N. Engl. J. Med. 321:574 (1989)]. In another embodiment, polymeric materials can be used [see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)]. In yet another embodiment, a controlled release system can be placed in proximity of the therapeutic target, i.e., the tissue of interest, thus requiring only a fraction of the systemic dose [see, e.g., Goodson, in Medical Applications of Controlled Release, supra, vol. 2, pp. 115-138 (1984)]. Preferably, a controlled release device is introduced into a subject in proximity of the site of a tumor.

Other controlled release systems are discussed in the review by Langer [Science 249:1527-1533 (1990)].

A subject in whom administration of the agent is an effective therapeutic regimen for retarding or overcoming multidrug resistance is preferably a human, but can be any animal, preferably a mammal. Thus, as can be readily appreciated by one of ordinary skill in the art, the methods and pharmaceutical compositions of the present invention are particularly suited to administration to any animal, particularly a mammal, and including, but by no means limited to, domestic animals, such as feline or canine subjects, farm animals, such as but not limited to bovine, equine, caprine, ovine, and porcine subjects, wild animals (whether in the wild or in a zoological garden), research animals, or for veterinary medical use.

In yet another aspect of the present invention, provided are pharmaceutical compositions of the above. Such pharmaceutical compositions may be for administration for injection, or for oral, pulmonary, nasal or other forms of administration. In general, comprehended by the invention are pharmaceutical compositions comprising an agent in an amount effective for disrupting the acidification of an intracellular vesicular compartment of the tumor cell and/or for disrupting the vesicular transport mechanism of an intracellular vesicular compartment of the tumor cell. Such agents can be administered with pharmaceutically acceptable diluents, preservatives, solubilizers, emulsifiers, adjuvants and/or carriers. Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. The compositions may be prepared in liquid form, may be in dried powder, such as lyophilized form. Alternatively, the agent can be administered in a pill form.

### EXAMPLE 1

### Effect of Cellular pH on MDR

Materials and Methods

Cells: NIH3T3 cells were grown at 37° C. in Dulbecco's Modified Eagle's Medium (DMEM) (Gibco Labs, MD) with 10% fetal calf serum (FCS) (Gemini Bioproducts, Inc, CA). NIH3T3 cell lines that were transfected with mdr-1 were supplemented with 100 nM vincristine sulfate. Myeloma cells (8226: the parental drug-sensitive line and DOX-40 the drug-resistant line) were grown in RPMI (Gibco, MD) with 10% FCS (Gemini Bioproducts). The drug-resistant line was supplemented with 100 nM doxorubicin-HCl (Calbiochem, CA). All media were supplemented with penicillin (Gibco Labs, MD), streptomycin (Gibco Labs, MD) and antimytopic (Gibco Labs, MD) with 2 mM L-glutamine (Gibco Labs, MD) and, unless indicated otherwise, maintained in 5% pCO₂.

Fluorescence and Confocal Microscopy: Fibroblasts (NIH3T3 cells) were grown on coverslips (VWR, 25 mm thickness 0.15 mm) which were placed in a Leiden coverslip chamber (Medical Systems, NY). Myeloma cells were adhered to the same coverslips with Cell-Tak (Collaborative Biomedical Products, Becton Dickinson, MA) according to the manufacturer's instructions. The chamber and solutions were kept at 37° C. Solutions equilibrated with ambient (0.033%), 2%, 5% or 10% CO₂ perfused at a constant velocity. Warmed air (at appropriate pCO₂) was perfused across the surface.

Fluorescence microscopy: The coverslip chamber with the cells was mounted on an Nikon Diaphot inverted microscope and illuminated with a 100 W Hg Lamp (Nikon) with a 97% neutral density filter. For quantification of cell-associated fluorescence, the chamber was mounted on a Zeiss Axiovert 135 inverted microscope with a 100 W Hg light source and a 97% neutral density filter and a Hamamatsu cooled CCD camera #C4880.

Confocal microscopy: The chamber was mounted on an inverted InSight Confocal Microscope (Meridian Instruments, Okemos, Mich.) which used an argon laser for excitation at 488 nm.

Chemicals: Daunomycin (Calbiochem, CA) and doxorubicin (Calbiochem, CA) were made as a 10 mM stock in water and stored at 4° C. SNARF1-AM (Molecular Probes, OR) was stored as a 20 mM stock in anhydrous DMSO (Aldrich, Wis.) and stored at −20° C.

pH measurement: The pH of cells was measured using SNARF1-AM (Molecular Probes, OR) according to the manufacturer's instructions. Fibroblasts grown on cover slip dishes in DMEM with 10% FCS were rinsed in DMEM without FCS, and then incubated in DMEM with 10 μM SNARF1-AM for fifteen minutes. The cells were then placed on an InSight and excited at 488 nm with emission recorded at 570/30 nm and 630/lp nm. A pH calibration curve was constructed by rinsing the cells with 150 mM KCl with 6 μM nigericin and 50 mM sodium phosphate buffered to pH 6.6, 6.8, 7.0, 7.2, 7.4, 7.6, 7.8 and 8.0. Myeloma cells were harvested and then resuspended in medium without FCS. SNARF1-AM was added to a final concentration of 10 g/ml for fifteen minutes at 37° C. The cells were then placed in a dialysis bag (SPECTRAPOR, Fisher Scientific, MW cutoff 12,000-14,000, 1.6 cm diameter) suspended in a 200 ml beaker with RPMI. The RPMI in the beaker was maintained at 37° C. and kept aerated with an aquarium airstone with 0.03%, 2%, 5%, or 10% CO₂ in air. The stirred bathing medium could be changed to vary the concentrations of CO₂ or drugs in the dialysis bag. For measurement in a spectrofluorometer an emission scan was taken from 520-700 nm with excitation at 488 nm and 514 nm. The cells were calibrated, as described above for the fibroblasts, for both excitations and the results at each was compared. For measurement on a fluorescence activated cell-sorter (FACS, Becton-Dickerson FACSTAR ^{PLUS}, MA) the cells were pumped at 0.38 ml/min with an Ismatec peristaltic pump (Cole-Parmer, Ill.) and excited with an argon laser at 514 nm and emission was monitored with filters at 570/26 nm and 630/30 nm. For measuring daunomycin concentration the cells were excited at 488 nm and emission monitored at 570 nm.

Results

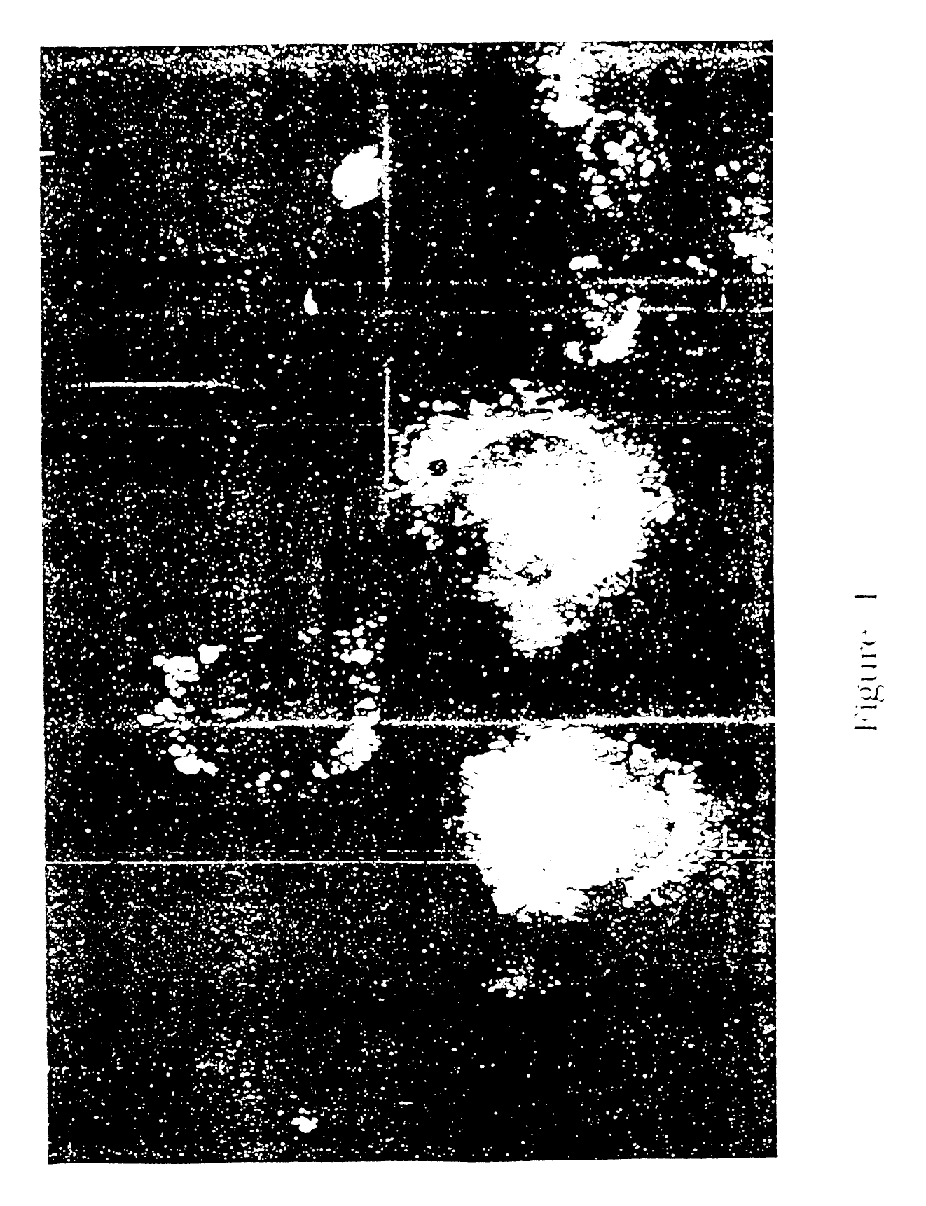

Daunomycin accumulates in cells: Daunomycin, a chemotherapeutic agent, fluoresces maximally at 595 nm when excited at 488 nm. These optical properties enable monitoring the drug in living cells. NIH3T3 fibroblasts were incubated in the presence of 5 M daunomycin for 30 minutes and examined on an inverted fluorescence microscope. Since the fluorescence spectrum of daunomycin is not affected by pH, the fluorescent images of increasing cytosolic daunomycin fluorescence reflect accumulation of the drug. The concentration of daunomycin in the cytosol (FIG. 1) is higher than in the surrounding media, with the highest concentration in the nucleoli and two of the major acidic compartments of the cell (trans Golgi and lysosomal), as has been previously reported [Weaver et al., Exp. Cell Res., 196:323-329 (1991)]. Similar patterns of intracellular accumulation were observed for cells incubated with doxorubicin and with several strains of NIH3T3 fibroblasts and with myeloma cells growing in suspension. Daunomycin binds DNA with great affinity and to a lesser extent RNA [Calendi et al., Biochim. Biophys., 103:25-49 (1965) and Doskocil et al., FEBS Letters, 37:55-58 (1973)]. Binding to tightly packed DNA in the chromatin results in quenching of the daunomycin fluorescence while binding to nucleoli yields fluorescent structures.

The pH is different in drug-sensitive and drug-resistant cells: The NIH3T3 fibroblasts and myeloma cells were loaded with SNARF1-AM, a dye whose fluorescence emission is pH-sensitive. When excited at 514 nm, its emission maximum is at 630 nm in a basic environment and at 570 nm when acidic. Ratioing of fluorescence emission is used as a quantitative measure of the pH, independent of cell volume or dye concentration. The pH of the myeloma cells, as measured in a FACS or spectrofluorimeter, was 7.1 for the drug-sensitive cells (8226) and 7.45 for the drug-resistant cells (DOX-40). The pH of the drug-sensitive NIH3T3 cells (mock transformed with a neomycin marker) was 6.8 while that of those transfected with mdr-1 was 7.25 as measured with a fluorescence confocal microscope.

Changing the pCO₂ rapidly and reversibly shifts cytosolic pH: To mimic the alkaline cellular pH shift that occurs in MDR, the pCO₂ was lowered. CO₂ quickly equilibrates across cellular membranes. The rapid activity of cytosolic carbonic anhydrase and the numerous cellular mechanisms to regulate bicarbonate exchange ensures that changes of pCO₂ rapidly affect cellular pH [Boron et al., Annu. Rev. Physiol., 48:377-388 (1986)]. NIH3T3 fibroblasts were loaded with SNARF1-AM and mounted on an inverted microscope. Changing the pCO₂ from 5% to 2% resulted in a rapid alkaline shift of intracellular pH in the fibroblasts from 6.8 to 7.2. This pH shift was reversible, changing the pCO₂ back to 5% returned the pH to 6.8. The basal pH value of 6.8 is somewhat more acidic than previously reported values for the NIH3T3 cells. However, in those experiments, pH was measured with the cells at an ambient pCO₂ of 0.033%. The pH rises as the pCO₂ is changed from 5% to 0.033% [Lin et al., Am. J. Physiol., 258:C132-C139 (1990)]. The results are consistent with published measurements in 5% pCO₂ [Gillies et al., Proc. Natl. Acad. Sci. USA, 87:7414-7418 (1990)].

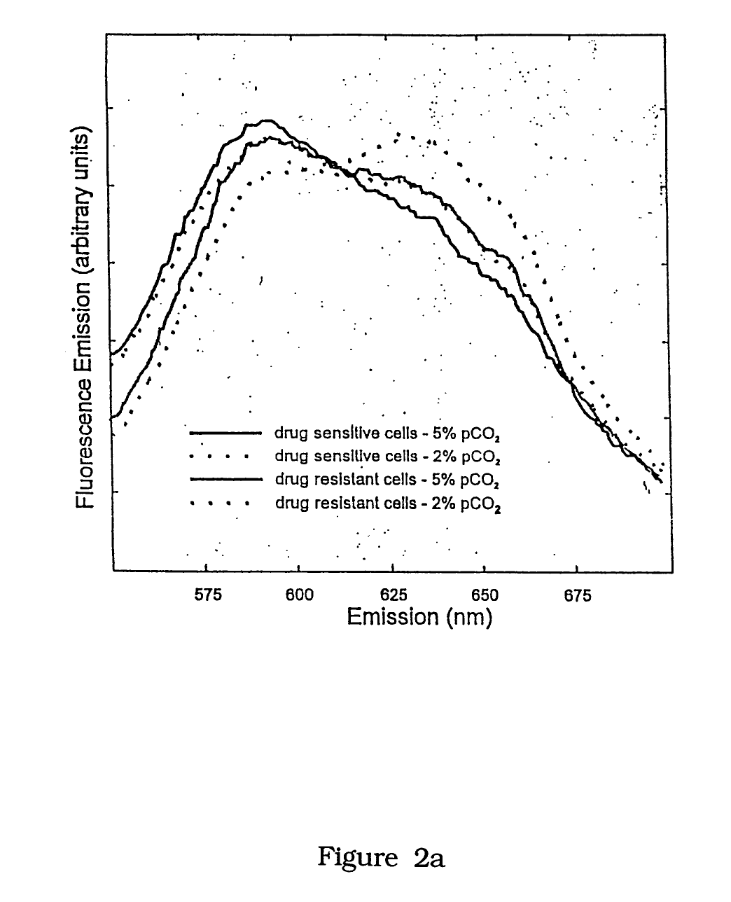

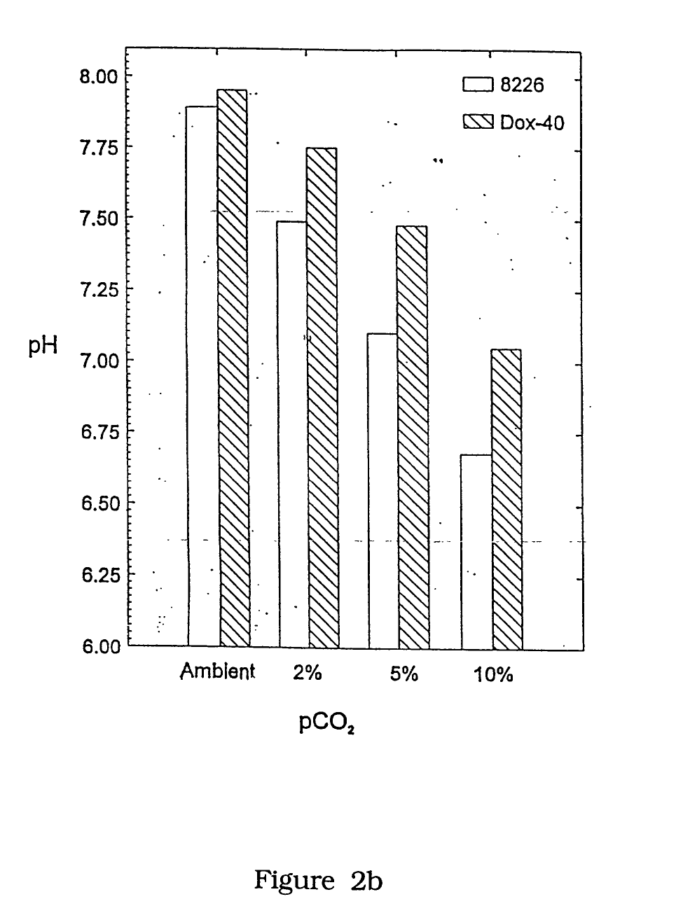

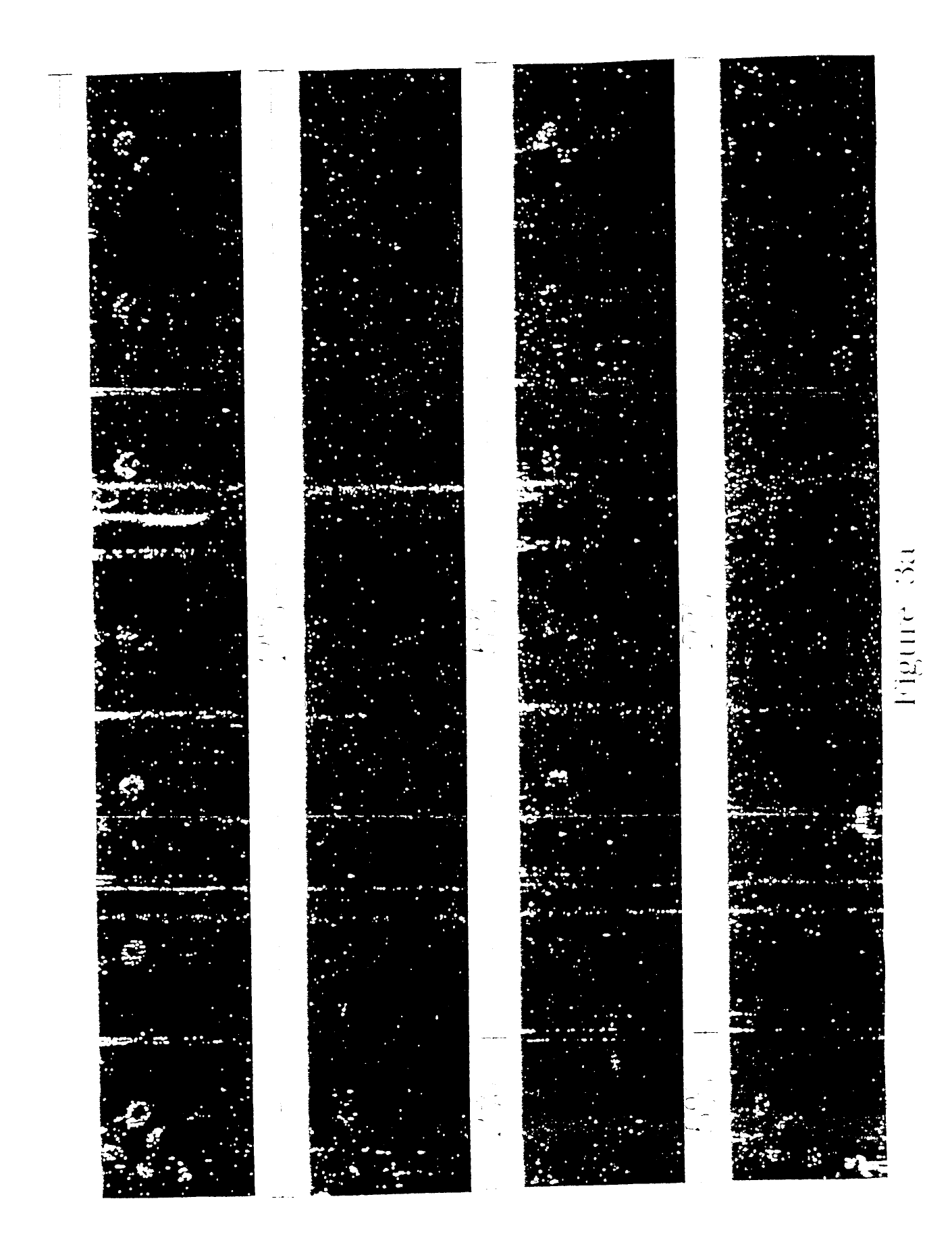

The pH of myeloma cells, grown in suspension, was examined in both a FACS and in a spectrofluorometer. The emission spectrum of SNARF1 in drug-sensitive cells (black line in FIG. 2a) and MDR cells (FIG. 2a gray line) is shown at a pCO₂ of 5% (solid line) or pCO₂ of 2% (dashed line). The pH of sensitive cells incubated with a pCO₂ of 2% (measured as the ratio of the emission at 630 nm to 585 nm, FIG. 2a dashed black line) was indistinguishable from the pH of the resistant cells at a pCO₂ of 5% (solid grey line). This demonstrates that varying pCO₂ can be used to shift the pH of the drug-sensitive cells to a value as alkaline as the resistant cells. Likewise, the pH of the drug-sensitive cells in 5% CO₂ (FIG. 2b) was comparable to the pH in resistant cells at a pCO₂ of 10% (FIG. 2b). Alternatively, the resistant cells could have their intracellular pH shifted to that of the more acidic sensitive cells.

The pH of the drug-sensitive myeloma cells was 7.1 and that of the drug-resistant cells in 5% pCO₂ was 7.45. The pCO₂ was modified in the following manner: 5%, 2%, 0.03%, 2%, 5%, 10%, 5%. The pH values were measured for each level of pCO₂. At a lower pCO₂, the intracellular pH was more alkaline and at higher pCO₂ more acidic. At a pCO₂ of 2%, the pH was 7.45 for the sensitive cells and 7.75 for the resistant cells. This was accompanied by a shift of only 0.04 pH units in the extracellular pH. Cells at 0.03% pCO₂ demonstrate pH of 7.85 (sensitive) and 7.9 (resistant). Returning the cells to a pCO₂ of 5% caused the pH to rapidly revert to the starting level. Further, increasing the pCO₂ to 10% caused an increased acidification to 6.65 (sensitive) and 7.05 (resistant). This sequence of cycling pCO₂ was repeated each time yielding the same intracellular pH values shown in FIG. 2b.

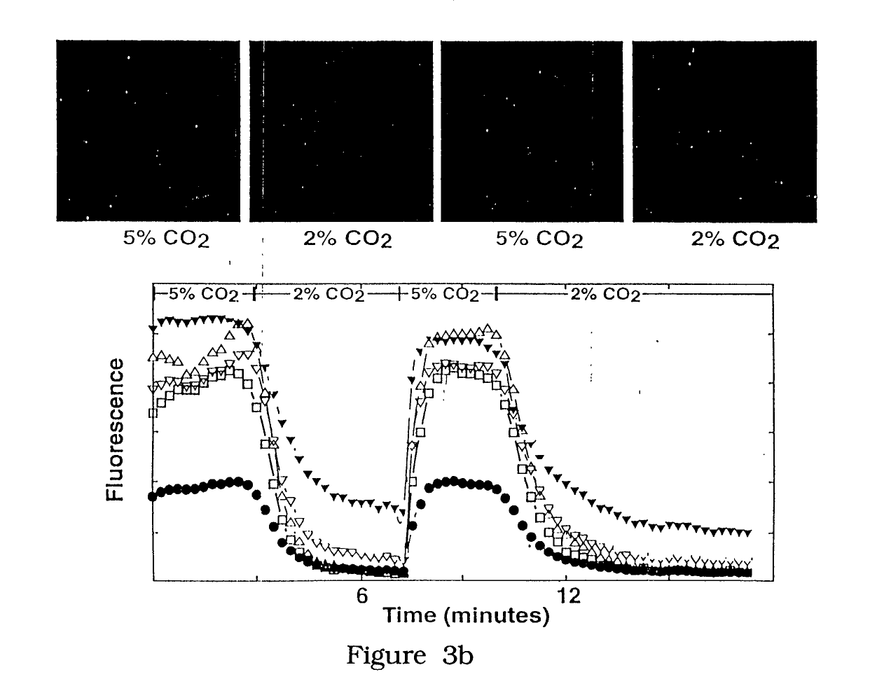

Changing pCO₂ rapidly and reversibly shifts intracellular daunomycin fluorescence: NIH3T3 cells at 5% pCO₂ were incubated with 5 μM daunomycin until the intracellular levels were approximately at a steady-state (FIG. 3a, red background). The pCO₂ perfusing the solution was shifted from 5% to 2% (FIG. 3a, blue background). The daunomycin fluorescence rapidly decreased in the cells. Upon returning the cells to 5% pCO₂ (red background), the daunomycin fluorescence increased to its starting level. The pattern remained unchanged upon repeated cycling between 2% and 5% pCO₂. The intracellular daunomycin fluorescence was quantified for a number of cells (FIG. 3b). In all cases, the cellular fluorescence decreased when the pCO₂ was lowered (more alkaline pH) and the fluorescence increased when the pCO₂ was increased. These changes were rapid, repeatable and reversible.

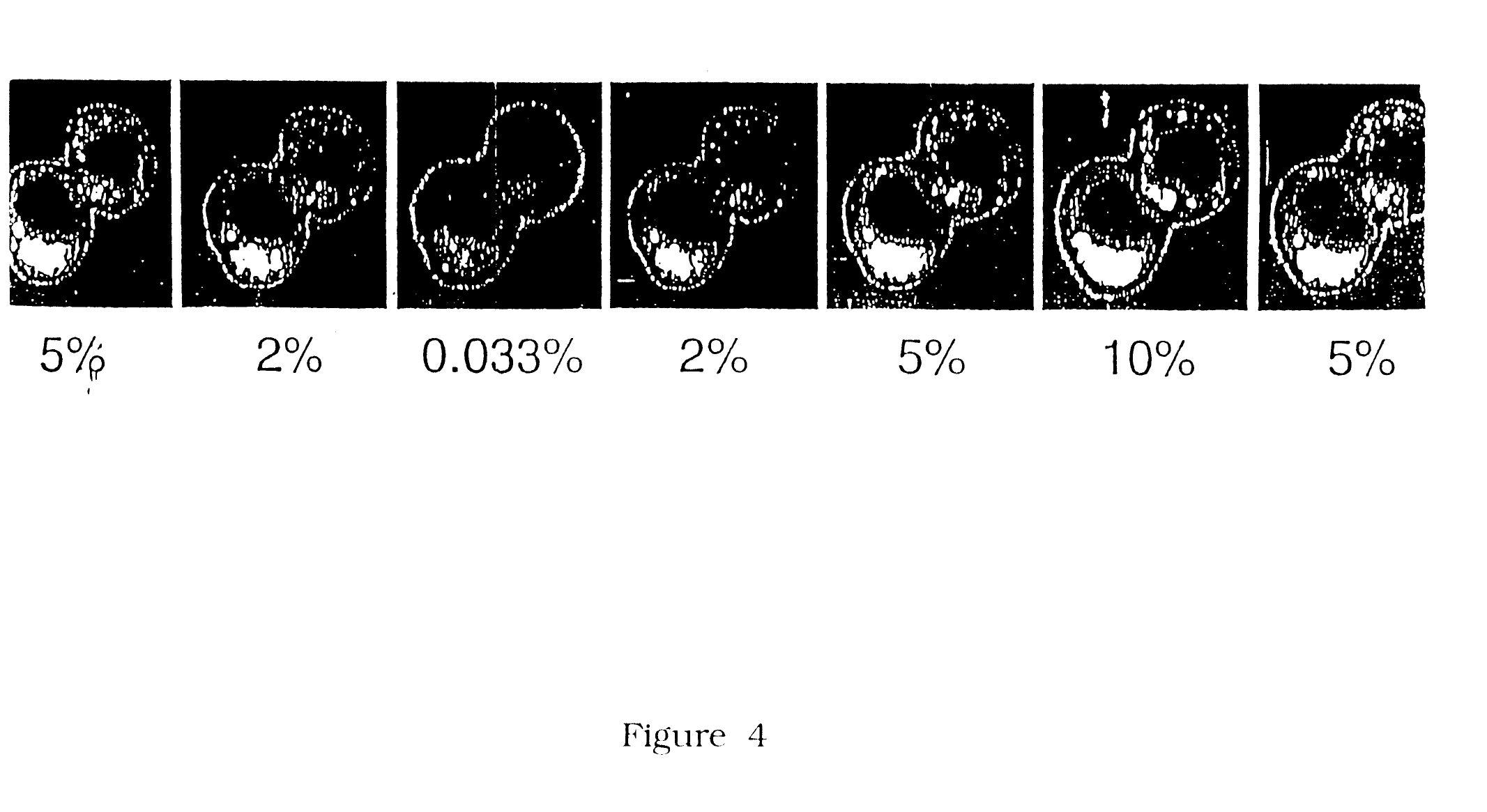

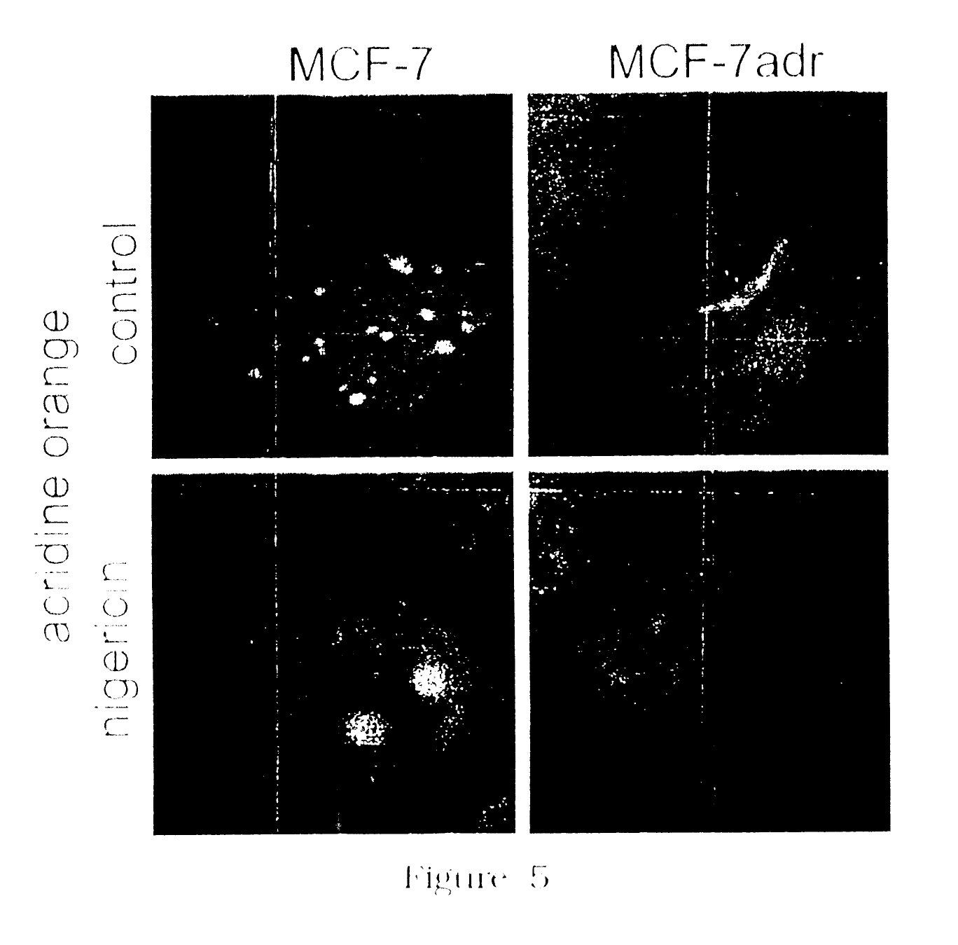

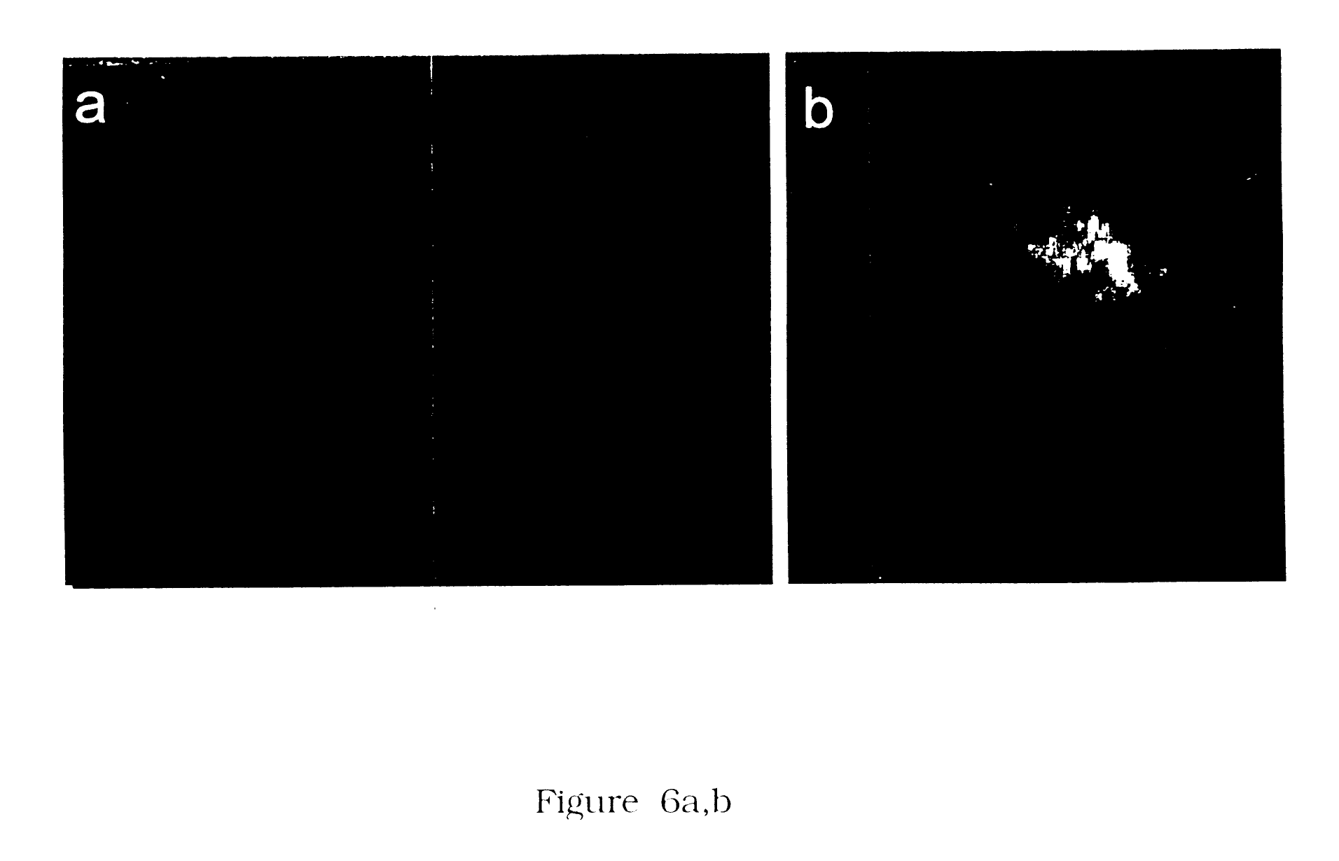

The experiment was repeated with both drug-sensitive (8226) and resistant (DOX-40) myeloma cells with similar results. Cells were loaded with 20 M daunomycin in medium equilibrated with 5% pCO₂. While monitoring the daunomycin fluorescence, the pCO₂ was sequentially shifted to 2%, 0.03%, 2%, 5%, 10% and 5% (FIG. 4a). This cycle was repeated. The cellular daunomycin fluorescence decreased when the pCO₂ was lowered (which shifts the pH alkaline) and increased when the pCO₂ was raised. These changes were completely reversible and occurred at the same rate in all intracellular compartments.

Similar reversible increases of cellular drug levels were also observed when the pH was transiently shifted alkaline with 20 mM NH₄Cl for 2 minutes. Reversible increases of cytosolic drug levels were observed when the pH was transiently shifted acidic with 2.5 mM NaN₃ for 2 minutes.

Discussion

Fluorescent chemotherapeutic agents accumulate in tumor cells (see FIG. 1). This could be a consequence of decreased drug influx, increased intracellular trapping and/or increased drug efflux. There are two general mechanisms for drug transport: active and passive. An active transport model for MDR has been proposed based on the observations that transport is blocked by metabolic inhibitors such as azide and that transport is associated with the expression of the P-glycoprotein, an ATP binding protein which is a member of a family of membrane transporters.

The passive diffusion models are based on the observation that these drugs are sufficiently hydrophobic to cross membranes. The asymmetric distribution of the drugs is assumed to be the consequence of an asymmetry of chemical potential (such as pH, voltage and ionic concentrations). For example, the higher rate of aerobic glycolysis in tumors and the hypoxic conditions surrounding cells within a tumor mass cause an acidic environment [Warburg et al., Science, 123:309-314 (1956)]. This increased proton concentration has two effects. First, the drugs that are weak bases will be protonated and trapped in the cytosol. Second, the binding of each of these drugs to their cytosolic targets, such as tubulin [Weisenberg et al, Biochemistry, 9:4110-4116 (1970) and Na et al., Archives of Biochemistry and Biophysics, 182:147-154 (1977)] or DNA [Zunino et al., Chem. Biol. Interact., 24:217-225 (1979); Zunino et al, Biochim. Biophys. Acta, 476:38-46 (1977); Zunino et al., Biochim. Biophys. Acta, 277:489-498; DiMarco et al., J. Cell Biol., 27:545-550 (1965) and Calendi et al., Biochim Biophys. Acta, 103:2549 (1965)], has an acidic pH optimum. Conversely, an increased pH both decreases intracellular drug accumulation and reduces binding to intracellular targets.

Passive transport of drugs in conjunction with a trapping mechanism is consistent with a number of independent observations. First, in simple systems such as red blood cell ghosts [Dalmark et al., The Journal of General Physiology, 78:349-364 (1981)] and phospholipid vesicles [Mayer et al., Biochim. Biophys. Acta, 857:123-126 (1986)] the transmembrane distributions of these drugs is determined by the pH. Second, the cytosolic pH of tumor cells increases with increased MDR [Keizer et al., J. Natl. Cancer Inst., 81:706-709 (1989)]. Third, transfection of cells with the P-glycoprotein causes an alkaline shift of cytosolic pH [Thiebaut et al., J. Histochem. Cytochem., 38:685-690 (1990)]. Fourth, verapamil, which reverses MDR, partially reverses this shift of cytosolic pH [Keizer et al., J. Natl. Cancer Inst., 81:706-709 (1989)]. Verapamil increases the concentrations of anti-cancer drugs even in cells which do not express P-glycoprotein [Nygren et al., Br. J. Cancer,64: 1011-1018 (1991) and Gruber et al., Leuk. Res., 17:(353-358 (1993)]. Fifth, drug influx is slower in resistant cells [Dano et al., Biochim. Biophys. Acta, 323:466-483 (1973); Ling et al., J. Cell Physiol., 83:103-116 (1978) and Beck et al., Mol. Pharmacol., 24:485-492 (1983)] which is consistent with differences in the rate of trapping and inconsistent with an active efflux model. Sixth, drugs which acidify the cytosol such as amiloride reverse MDR [Epand et al., Br. J. Cancer, 63:247-251 (1991)].

As shown, the passive trapping hypothesis can account for changes in cellular accumulation of chemotherapeutic agents that are weak bases. None are negatively charged but some, such as colchicine, are neutral. Each of these drugs has an intracellular target. Binding of colchicine to its target, the extremely acidic carboxy terminus of tubulin [Mukhopadhyah et al., Biochemistry, 29:6845-6850 (1990)] is pH dependent with an optima of pH 6.7-6.8 [Wilson, Biochemistry, 9:4999-5007 (1970)]. Any alkaline shift of the pH decreases the binding of colchicine and could protect the cell from this chemotherapeutic agent.

Multiple forms of non-P-glycoprotein MDR have been observed. The passive transport theory predicts that each affects a common feature—regulation of cellular pH. One protein responsible for non-P-glycoprotein-mediated MDR has recently been cloned and demonstrated to be a vacuolar H⁺-ATPase subunit [Ma et al., Biochem. Biophy. Res. Commun., 182:675-681 (1992)]. Other mechanisms for MDR may use pH to affect drug distribution either by selective sequestration, i.e. drug uptake by lysosomes, or modifications in the secretory pathway [Beck et al., Biochem. Pharm., 36:2879-2887 (1987)]. Consistent with this hypothesis is the observation of an increase in non-specific adsorptive endocytosis in anthracycline- and vinca alkaloid-resistant cells [Sehested et al., J. Natl. Cancer Inst., 78:171-179 (1987)], as well as an increase in membrane traffic in daunomycin-resistant cells [Sehested et al., Br. J. Cancer, 56:747-751 (1987)]. In drug-resistant cells, there is a significant rate of exocytosis of lysosomal enzymes, suggesting a modification of the endocytic pathway. Furthermore, an enhanced rate of exocytosis of vesicles containing a H⁺-ATPase could be a means by which cytosolic pH is raised, as has been observed in plant and animal cells [van Adelsberg et al., J. Cell Biol., 102:1638-1645 (1986) and Hager et al., Planta, 185:527-537 (1991)].

These results demonstrate the passive trapping model is sufficient to account for the enhanced sensitivity of tumors to anti-cancer drugs and the decreased sensitivity in MDR. When the pH of drug-sensitive cells is shifted to the level observed in drug-resistant cells, they no longer accumulate chemotherapeutic agents. Likewise, when drug-resistant cells are shifted to the level observed in drug-sensitive cells, they accumulate chemotherapeutics. Although, our results neither directly prove nor disprove the hypothesis that the P-glycoprotein is an ATP-driven drug efflux pump or flippase they demonstrate the existence of alternate pathways for MDR.

As described in detail above, there are a number of potential therapeutic implications from this work. If tumor cells are compromised in their ability to regulate pH, they may be more susceptible than healthy cells to pharmacological approaches that modify pH regulation. Thus, approaches that affect pH may potentiate the effects of the chemotherapeutic agents and, in this manner, reverse MDR.

### EXAMPLE 2

### Defective pH Regulation of Acidic Compartments in MCF-7 Cells is Normalized in MCF-7 ADR Cells

Introduction

Cancer cells are more sensitive to chemotherapeutic drugs than normal cells. The development of drug resistance in tumors treated with chemotherapeutics is accompanied by changes in cell physiology. This includes overexpression of numerous cellular proteins, changes in the subcellular distribution of the chemotherapeutics and an alkaline shift of cellular pH. It has been suggested that the alkaline shift could be causally related to drug-resistance. Most of the chemotherapeutics are weak bases with pKa's of 7-8. Thus, they would be expected to accumulate in tumor cells which are more acidic than normal, or drug-resistant cells [Simon Proc. Natl. Acad. Sci. USA, 91:1128-1132 (1994)]. The pH difference between drug resistant tumor cells and their parental cell lines is sufficient to quantitatively account for the decreases in drug-accumulation observed in drug-resistant cells [Simon et al., Proc. Natl. Acad. Sci. USA, 91:1128-1132 (1994)]. However, it has recently been reported for one particular cell line, the MCF-7 breast cancer cell, that the pH difference is only 0.2 between the drug-resistant and drug-sensitive cells [Altenberg, Proc. Nat. Acad. Sci. USA. 90: 9735-9738 (1993)]. This is too small a pH gradient to account for the differences in cellular drug accumulation.

Described herein are the structural and functional alterations within MCF-7 and MCF-7adr cell lines that are correlated with either enhanced drug sensitivity or resistance. These differences are observed as changes in: (a) the organization of the secretory compartment, TGN, (b) the activity and organization of the endosomal/pericentriolar recycling compartments, (c) secretion of lysosomal enzymes, and (d) the cytoplasmic and vesicular pH. Each of these cellular changes could be affected by a disruption of the acidic gradients in the TGN and recycling endosome.

In agreement with the previous report [Altenberg, Proc. Nat. Acad. Sci. USA. 90: 9735-9738 (1993)], the total cellular difference of pH between the MCF-7 and MCF-7 adr cells was only 0.3 pH units. However, there were substantial differences in subcellular pH. Specifically in the MCF-7 cells, there is a complete absence of subcellular pH gradients. In contrast, the drug-resistant MCF-7adr cell line has restored pH gradients. The cytosolic pH of the MCF-7adr cells is significantly more alkaline than the cytosolic pH of the MCF-7 cells and the organellar pH is significantly more acidic in the MCF-7adr cells. Thus, as with all other drug-resistant cell lines, chemotherapeutic drugs are excluded from the cytosolic compartments by pH gradients. Drugs that reach the cytosol are trapped in the acidic secretory pathway and rapidly passed from the cell. Disrupting the pH gradients of the secretory pathway reversed the drug-resistance of the cells.

Experimental Procedures

ReagentsL Acridine orange was purchased from Aldrich (Milwaukee, Wis.). The fluorescent reagents, Bodipy-ceramide, the acetoxymethylesters of both carboxy SNARF and SNAFL-calcein, and Bodipy-lactalbumin, were from Molecular Probes (Eugene, Oreg.). Adriamycin was from Calbiochem (La Jolla, Calif.). Monensin and nigericin were from Sigma (St. Louis, Mo.).

Tissue culture: Cells were seeded and grown in Dulbecco Modified Eagle's (DME) media containing 10% fetal calf serum (phenol red free) in Lab-Ten culture chambers (Nunc, Naperville, Ill.) maintained in an incubator at 37° C. and 5% CO₂. Human breast cancer cells (MCF-7) and the adriamycin resistant line (MCF-7adr) were obtained from Dr. William W. Wells of the Department of Biochemistry, Michigan State University. The media for the MCF-7adr cells was supplemented with adriamycin (0.5 μg/ml). Cells were utilized 3-4 days following plating.

Confocal fluorescence imaging of intracellular pH and acidic compartments: Acridine orange (2 μg/ml media; 4 mg/ml stock in water) was added directly to the chambers and the cells were incubated with the dye at 37° C. for 30 minutes. Cells in the presence of acridine orange were then examined at room temperature with an Insight Bilateral Laser Scanning Confocal Microscope (Meridian Instruments, Okemos, Mich.). Excitation was at 488 nm (argon ion laser beam) and dual emission confocal images were sequentially recorded utilizing both a 530-30 band pass barrier filter (green fluorescence) and a 605 nm long pass barrier filter (red fluorescence). Acridine orange demonstrates a concentration dependent long wavelength shift in the fluorescence emission; it shows a red fluorescence when accumulated to a high concentration within acidic cellular compartments and a green fluorescence when bound at lower concentration to membranes and/or nucleic acids. Optical sections of the fluorescent sample were recorded at 0.5 micron intervals. Typical individual sections are presented to demonstrate the distribution of acridine orange within the cytoplasmic and vesicular compartments. The acetoxymethylester derivative of SNAFL-calcein (15 μg/ml) (Molecular Probes, Eugene, Oreg.) (a radiometric fluorescent probe for pH) was added to both MCF-7 and MCF-7adr cells. The ester linked fluorescent probe enters the cell passively where the esters are hydrolyzed by esterases located in the cytoplasm and intracellular vesicles. The SNAFL-calcein is then ionically trapped within the cytoplasm and vesicular compartments. The cells were incubated at 37° C. for 45 minutes and then examined with the Insight confocal fluorescence. Optical sections were obtained utilizing two different filter settings for emission (530-30 band pass barrier filter and 630 long pass filter) and a single excitation wavelength (488 nm) as previously described for carboxy SNARF-1 [Simon et al., Proc. Natl. Acad. Sci., 91:1128-1132 (1994)]. The pixel intensities obtained at the two different emission intensities were divided to obtain a ratio image of the internalized pH probe [Simon et al., Proc. Natl. Acad. Sci., 91:1128-1132 (1994)]. These images were then compared to standard curves that were obtained in the following manner. To obtain a quantitative relationship between emission ratios and pH, each SNAFL-calcein stained cell line was exposed to a buffer at a known pH containing nigericin/high K⁻ (18 (M150 mM KCl) [Simon et al., Proc. Natl. Acad. Sci., 91:1128-1132 (1994)]. This treatment equilibrates all the internal compartments of the cell to the pH of the incubating buffer. By sequentially changing the pH buffer that is bathing the cells, a pH curve was generated for each cell line that demonstrated the relationship between the SNAFL-calcein fluorescence emission ratio and pH. These values were then incorporated into a pH imaging routine that provides a direct read-out of pH values for individual intracellular compartments that are queried on the computer screen. Cells treated with monensin were exposed to the drug (10 μg/ml of media) for 30 minutes at 37° C. prior to labeling with SNAFL-calcein as described above. All cells were examined at room temperature.

Fluorescence labeling and confocal imaging of endosomal and secretory compartments: Bodipy-ceramide (Bodipy-Ceramide; Molecular Probes, Eugene, Oreg.) has been demonstrated to label Golgi compartments. Conversion of Bodipy-ceramide to Bodipy-sphingomyelin (in cis-Golgi) is associated with the movement of the newly synthesized fluorescent lipid to the trans-Golgi network (TGN). As the Bodipy-sphingomyelin concentration increases within the TGN and secretory vesicles, a long wavelength shift in fluorescence occurs that results in red fluorescent structures (TGN and secretory vesicles) against a green fluorescent background. Cells were incubated with Bodipy-ceramide (3 μg/ml) for 15 minutes at 37° C., washed once with fresh media and then examined in optical section at room temperature with confocal fluorescence microscopy. Excitation was at 488 nm and dual emission images were prepared utilizing the filter set described for acridine orange (FIG. 5). To examine internalization, Bodipy-lactalbumin (Bodipy-Lac, Molecular Probes, Eugene, Oreg.) was used as a fluid phase marker. Cells were incubated with Bodipy-Lac (2 mg/ml) for 90 minutes at 37° and then washed once with cold media and rapidly examined with confocal fluorescence microscopy (excitation at 488 nm (λex) and emission (λem) at 530 nm (using a 30 nm band bass filter)).

Cell viability assays: The media was removed 60 hr. after plating the cells and replaced with fresh media supplemented with various concentrations of adriamycin (Calbiochem, CA) and monensin (solubilized in DMF 0.1%) (Sigma, St. Louis). After 6 hr the media was removed, the cells rinsed, and then fed with fresh media not containing drugs. The cells were fed daily for three days and then the DNA content of the adherent cells was quantified fluorometrically by Hoechst 33258 fluorescence. Media was aspirated and the wells rinsed with Hanks Balanced Salt Solution (HBSS, phenol red free). The cells were sonicated in hypotonic media (0.1×HBSS) for 30 seconds. The homogenate from each well was collected and Hoechst 33258 was added to a final concentration of 1 μg/ml. Fluorescence was measured on an SLM Aminco-Bowman series 2 luminescence spectrometer with a λₑₓ of 356 nm and a λₑₘ of 492. Calf thymus DNA was used for calibration.

Results

The fluorescent pH sensitive probe carboxy SNARF-1 (acetoxymethylester form) [Whitaker et al., Anal. Biochem., 194:330-344 (1991)] (Molecular Probes, Eugene, Oreg.) was employed to measure the intracellular pH in both MCF-7 and MCF-7adr cells. This probe partitioned within the cytoplasm of both cell lines. The cytoplasmic pH for MCF-7 cells was 6.8±0.1 (10 cells, 3 separate confocal sections) and for MCF-7adr cells 7.1±0.1 (10 cells, 3 separate confocal sections) (Table 1) consistent with other published measurements reporting a more acidic cytoplasm for drug sensitive cells [Simon et al., Proc. Natl. Acad. Sci. USA, 91:3497-3504 (1994)]. The more acidic cytoplasmic pH measured in MCF-7 cells suggested that the drug sensitive cells were manifesting an aberrant regulation of intracellular pH that might be representative of other changes in pH within intracellular vesicular compartments. This was examined with acridine orange, a probe previously employed to determine the presence of acidic intracellular compartments [Barasch et al., Nature, 352:70-73 (1991)]. As observed in FIG. 5, MCF-7 cells have few orange stained vesicles within the cytoplasm (FIG. 5, top row left). In sharp contrast, intensely red stained vesicles are observed in MCF-7adr cells in both the pericentriolar region of the cytoplasm and dispersed throughout the cytoplasm (FIG. 5, top row right). Treatment of either cell type with nigericin (7.5 μM) eliminated fluorescent staining of both cytoplasmic vesicles and vesicles within the pericentriolar region (FIG. 5, second row).

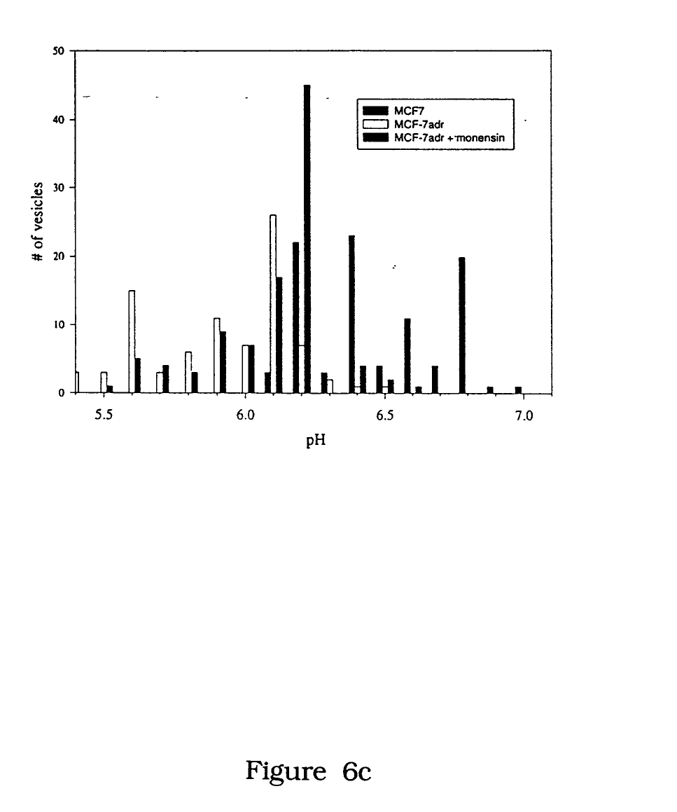

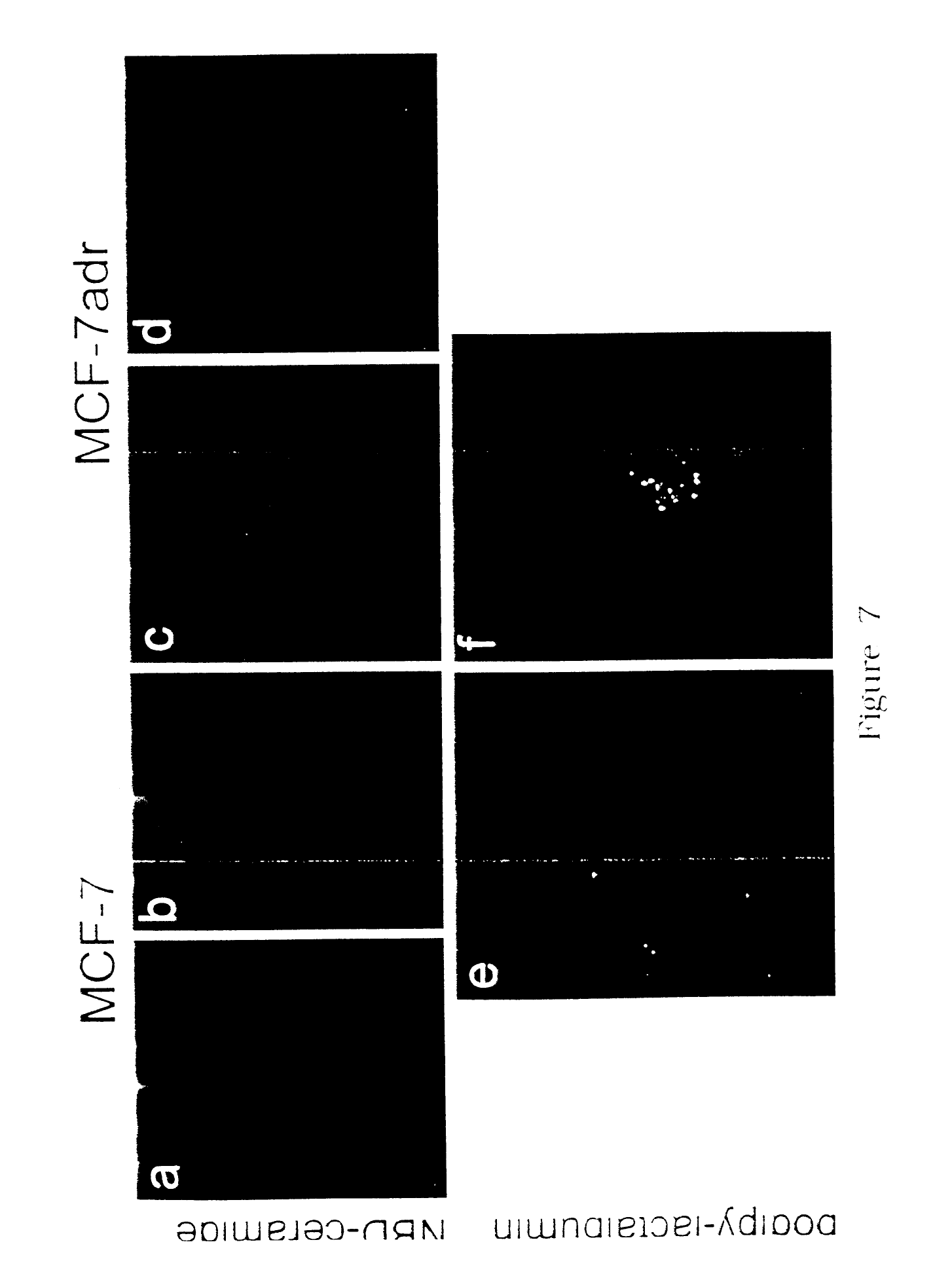

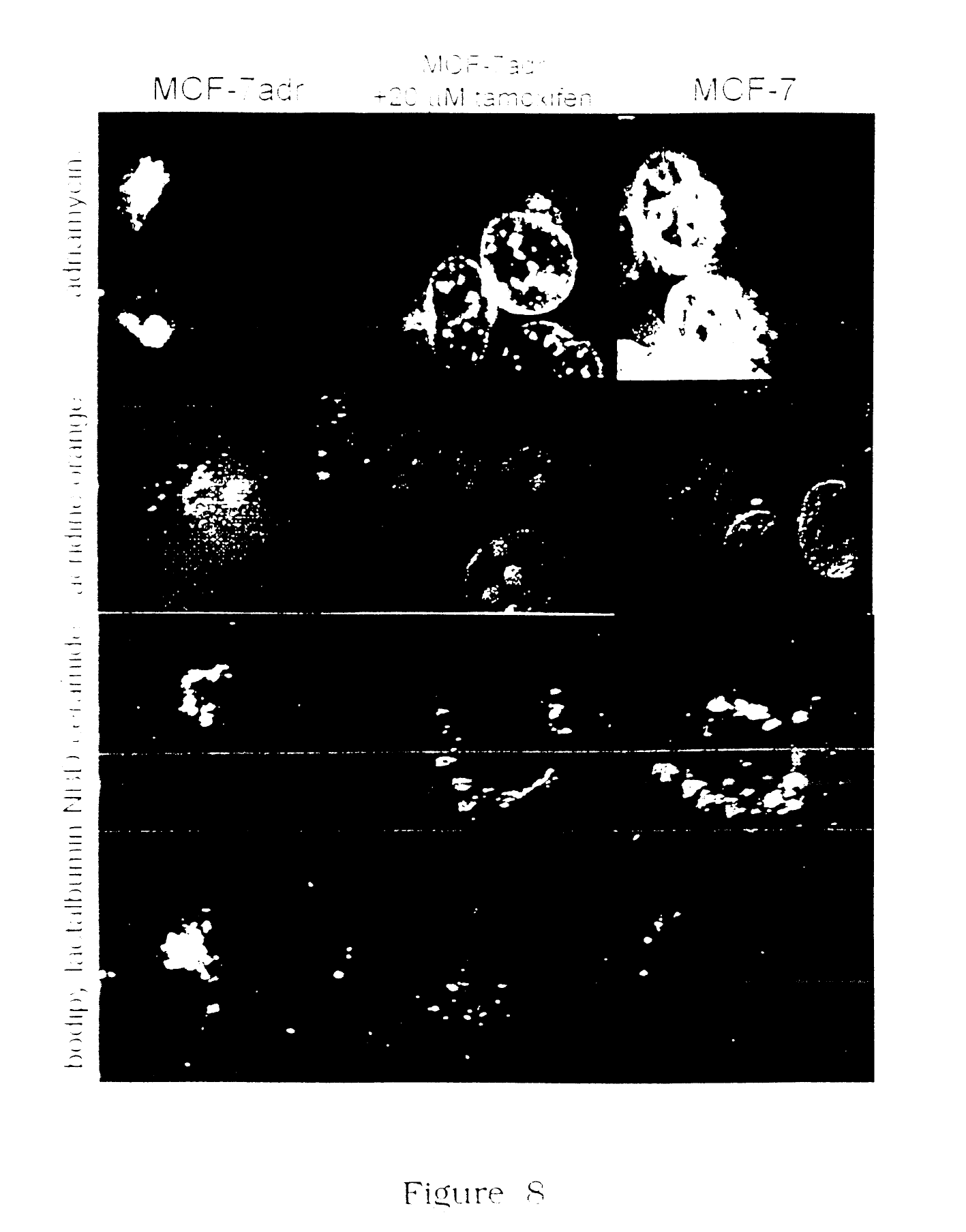

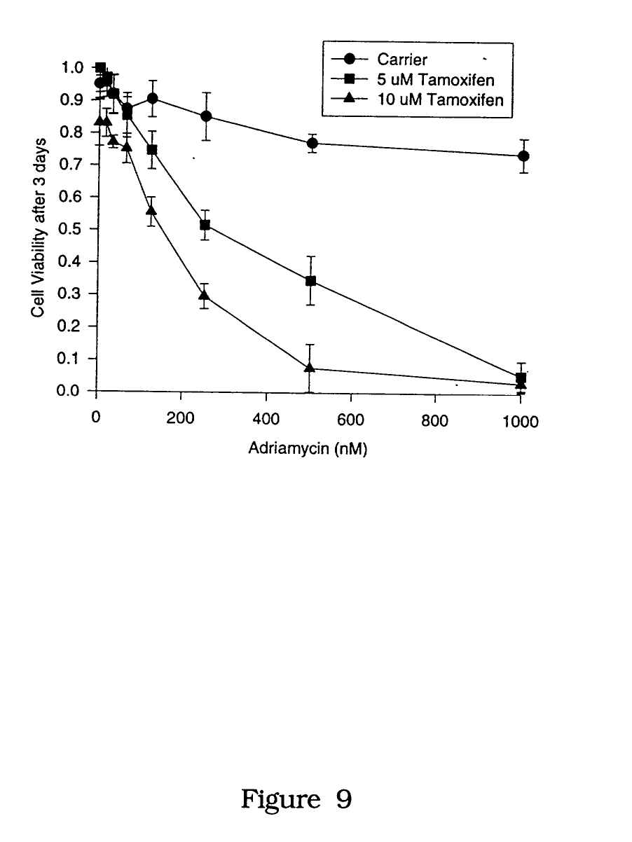

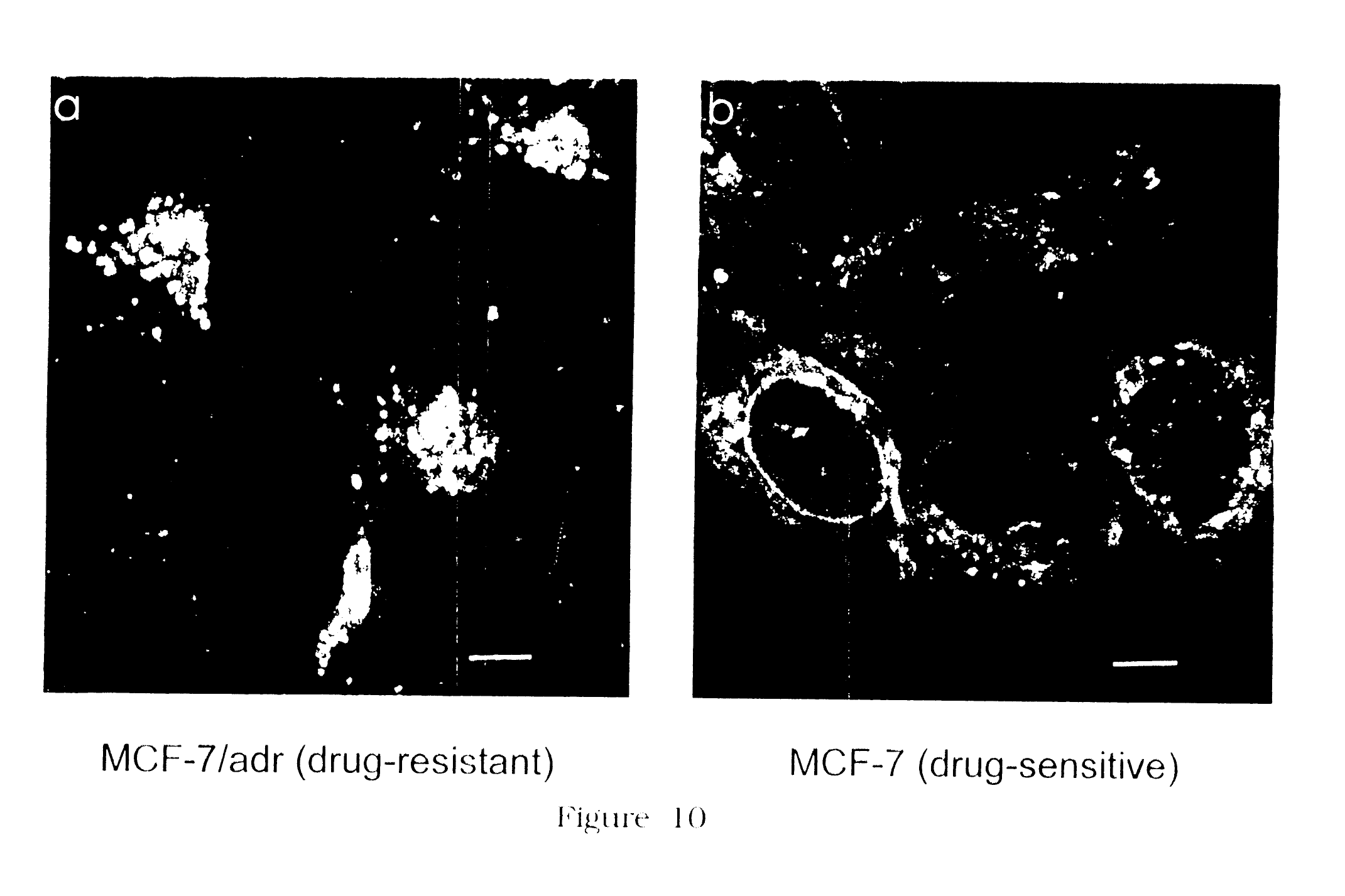

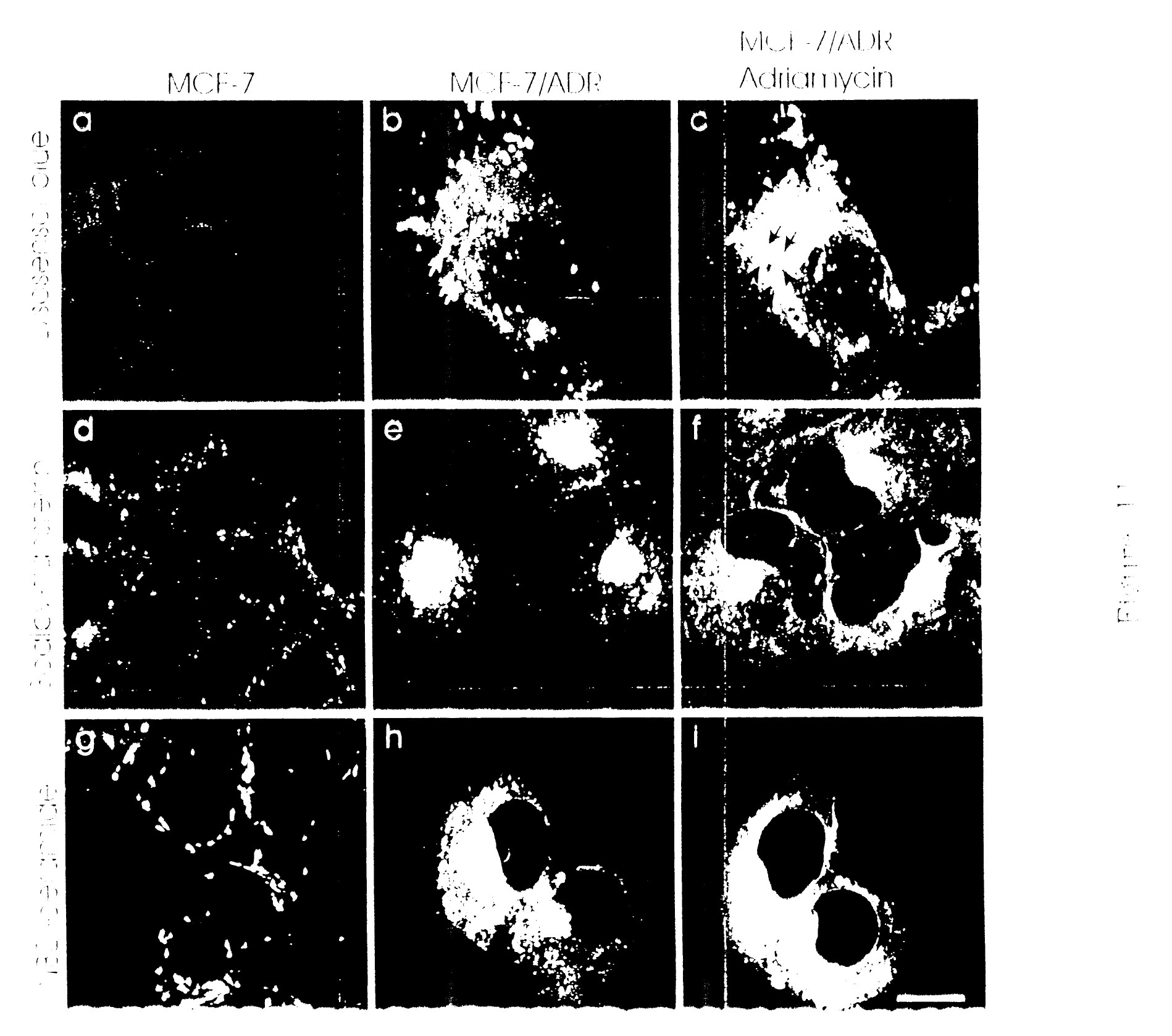

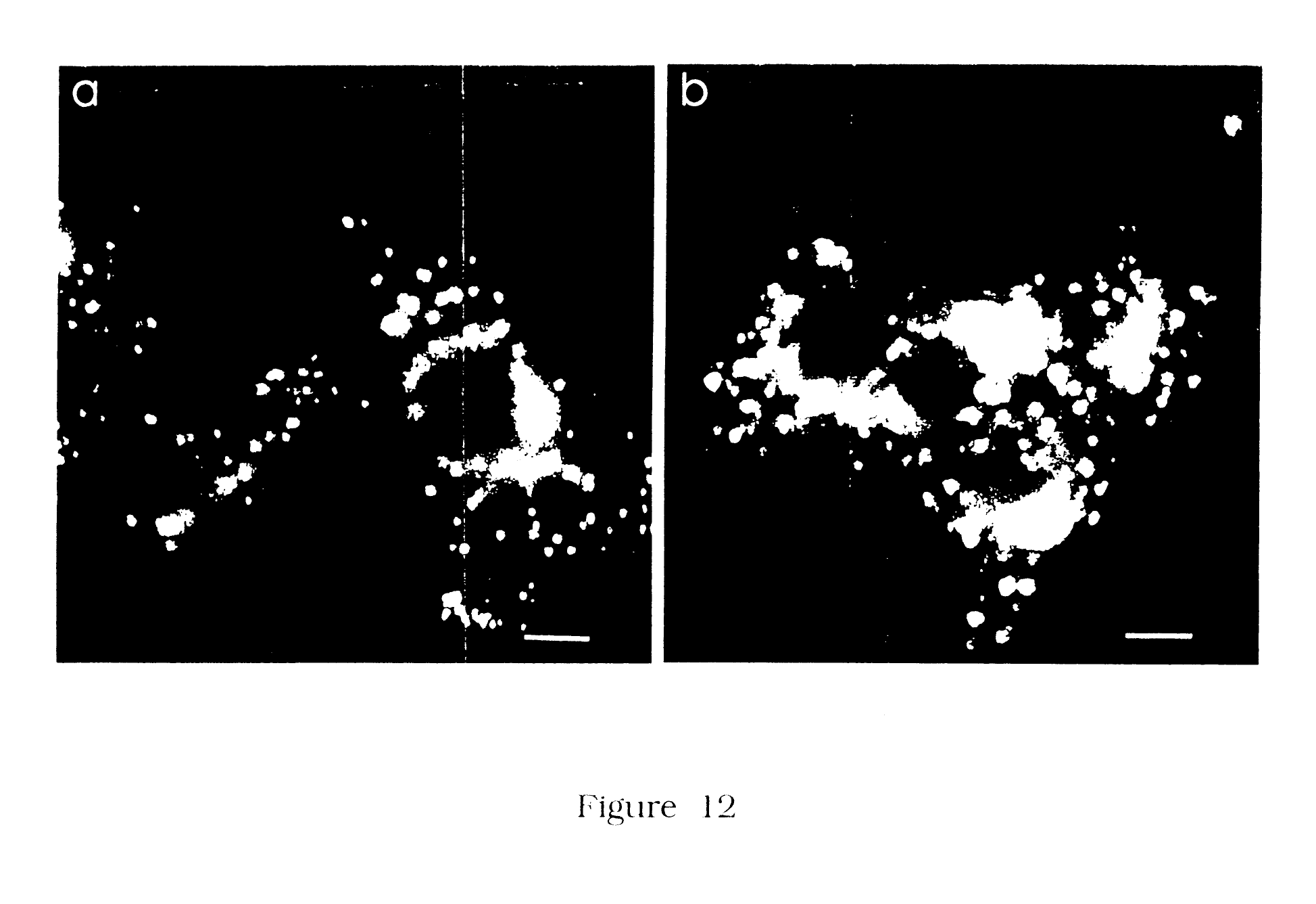

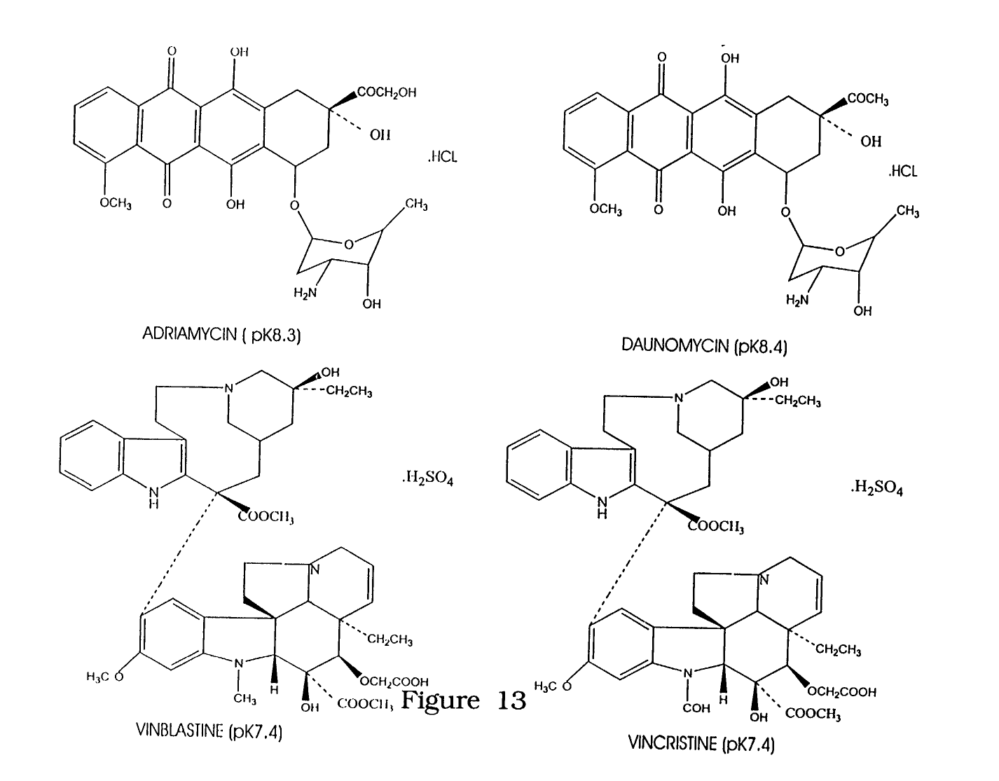

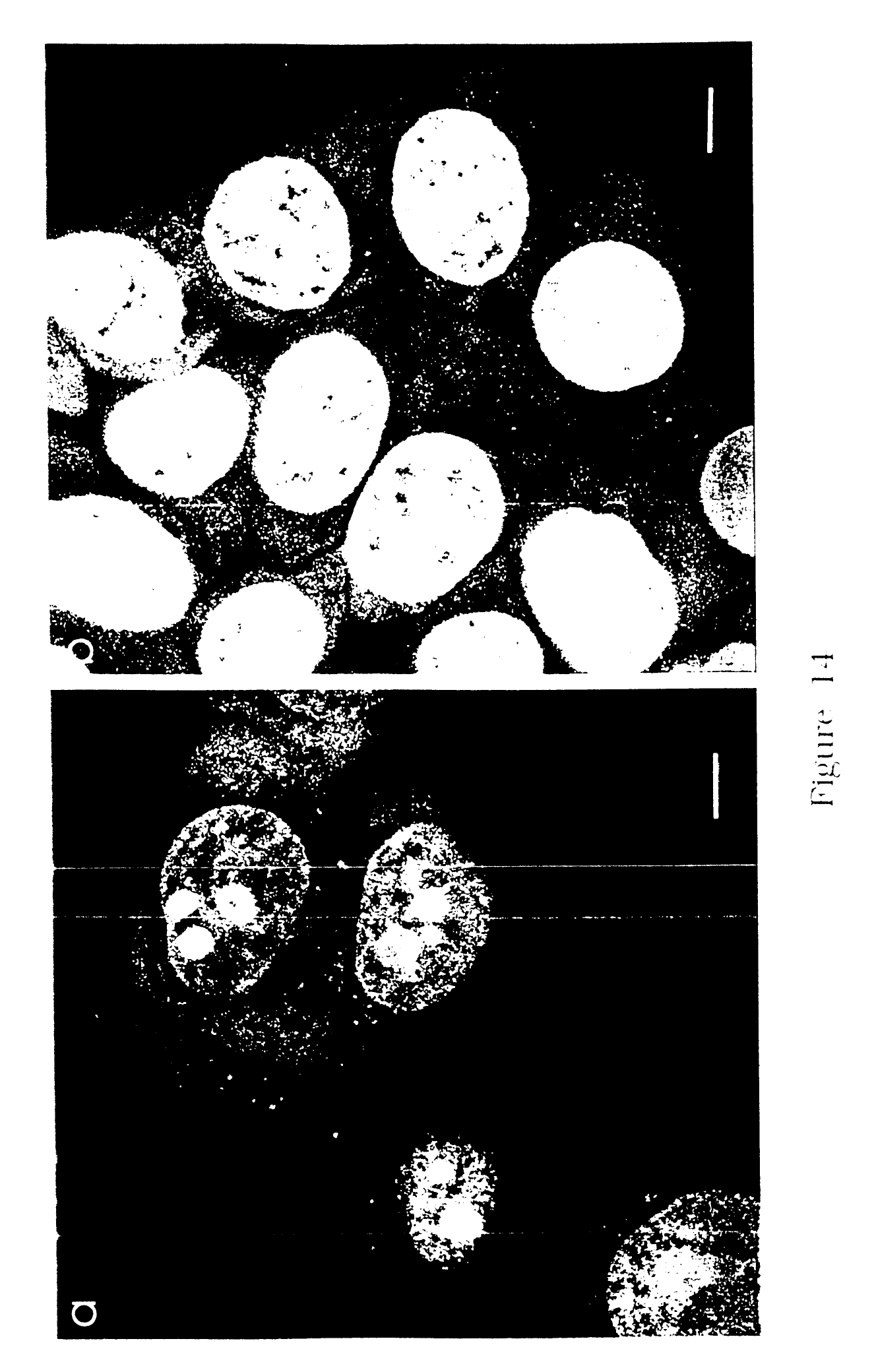

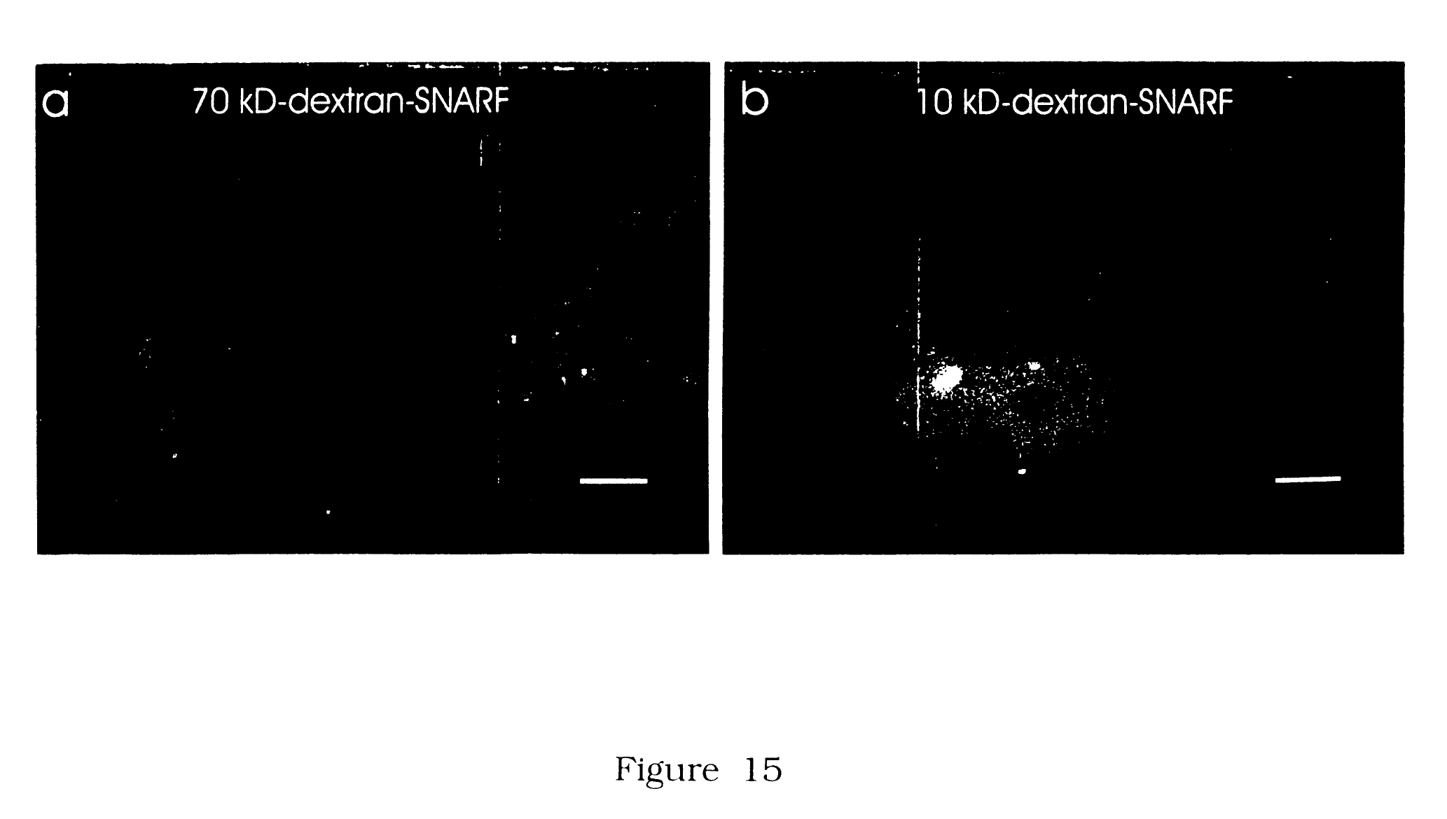

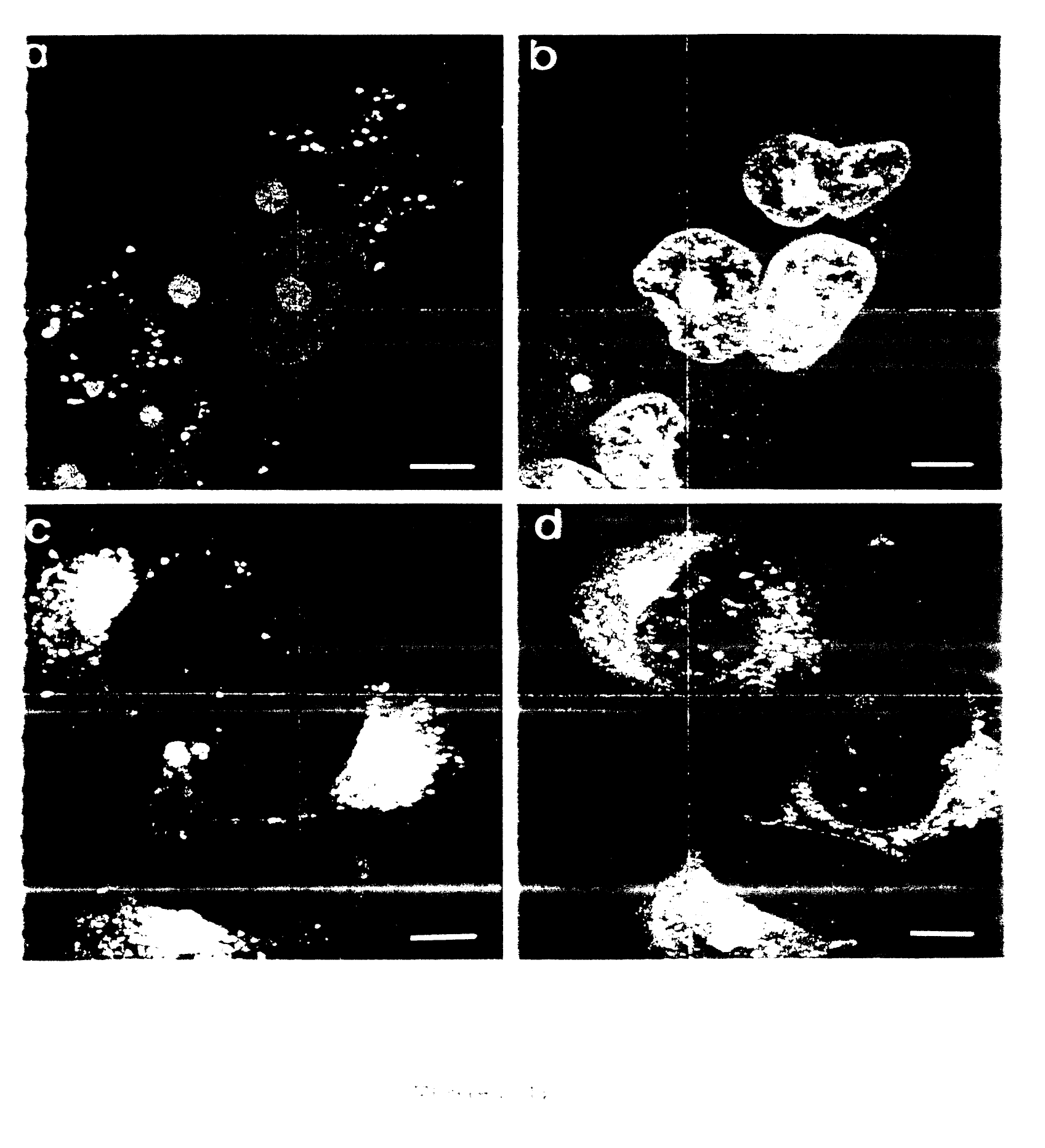

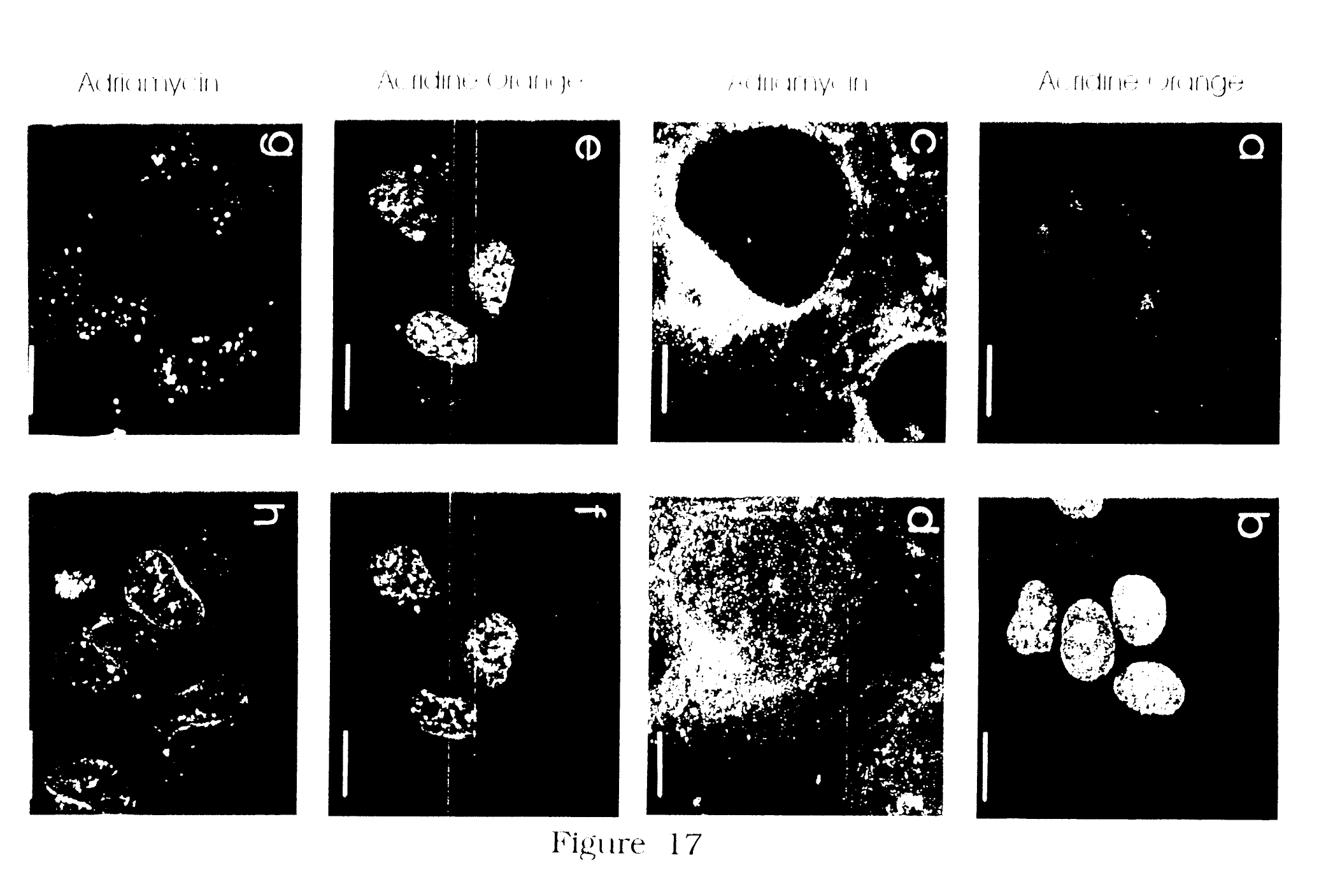

To quantify the pH in intravesicular compartments the fluorescent pH probe SNAFL-calcein (acetoxymethylester form) was used [Whitaker et al., Analytical Biochemistry, 194:330-344 (1991)] (Molecular Probes, Eugene, Oreg.). SNAFL-calcein was observed to differentially localize within the two cell types. In MCF-7 cells, the probe was shown to distribute predominantly in the cytoplasm of cells with accumulation in a few vesicles (FIG. 5, bottom row left). Again, in sharp contrast, little cytoplasmic labeling was observed in the MCF-7adr cells (FIG. 5, bottom row right), but considerable vesicular labeling was demonstrated particularly in the pericentriolar region. This was also observed for staining of MCF-7adr cells with acridine orange (FIG. 5, top row). To quantify the pH within these vesicular compartments, the fluorescence emission ratio of SNAFL-calcein was determined for individual vesicles within a population of MCF-7 and MCF-7adr cells (20 cells per cell type) [Simon et al., Proc. Nail. Acad. Sci. USA, 91:1128-1132 (1994)]. These values were then compared to a standard curve (see legend to FIG. 5). As shown in the pH histogram (FIG. 6, Table 1), the vesicular compartments are significantly more acidic in MCF-7adr cells than in the sensitive MCF-7 cells; further, the pH gradients between the cytoplasm and the lumenal compartments is considerably larger in MCF-7adr cells (Table 1).

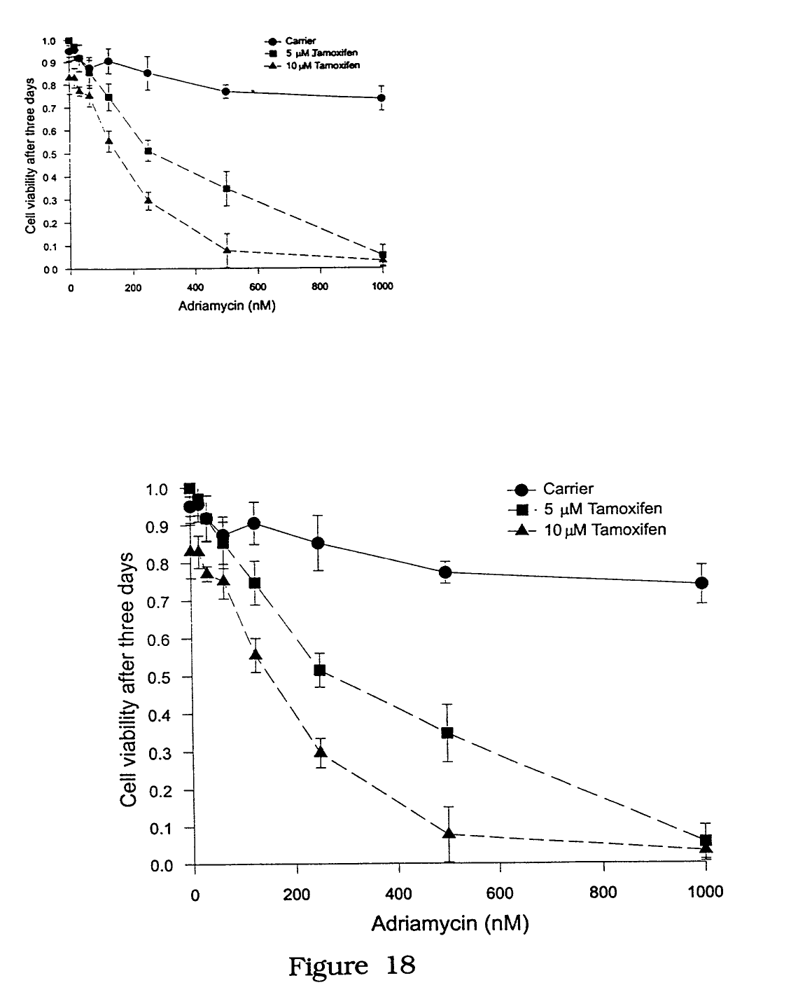

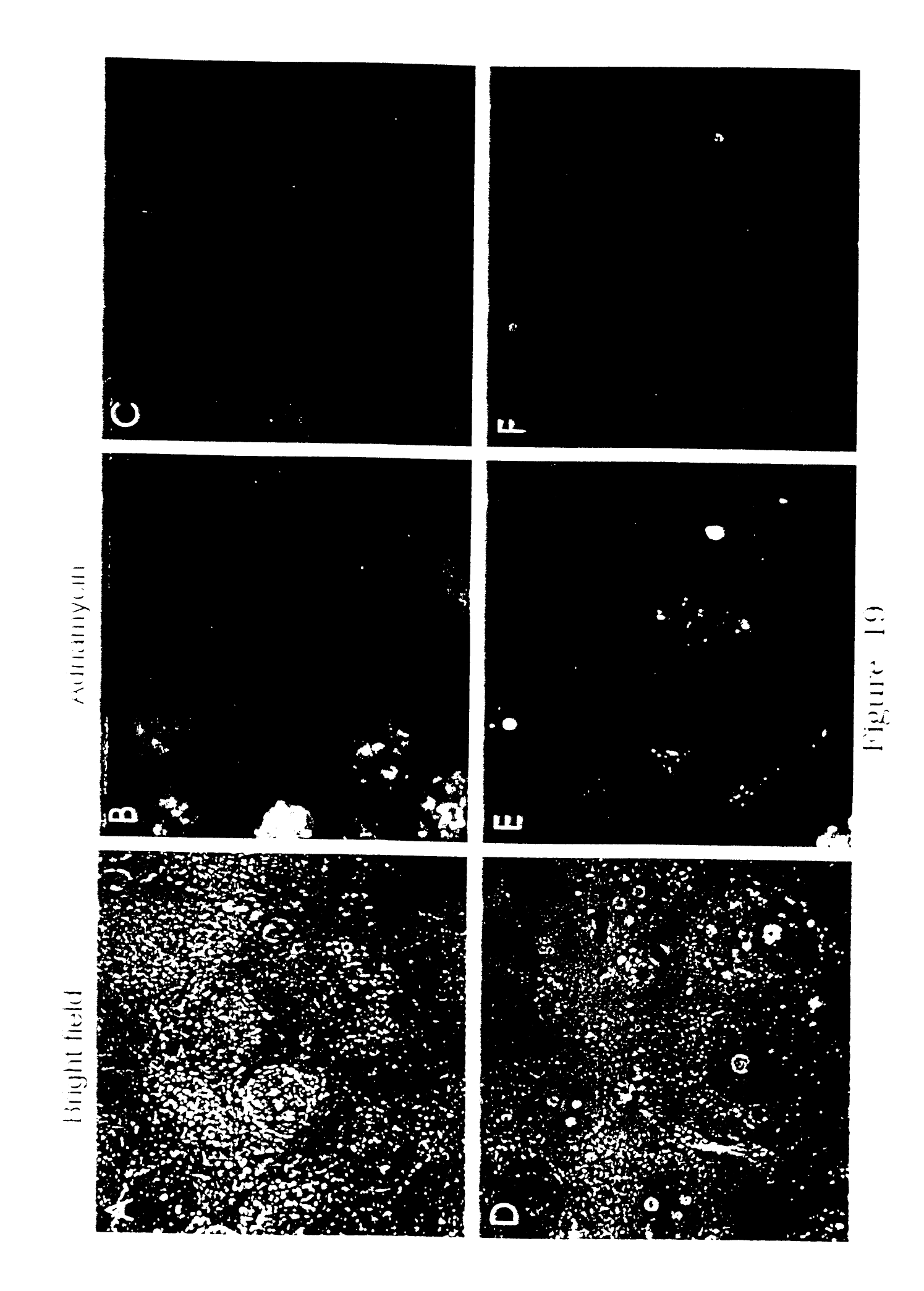

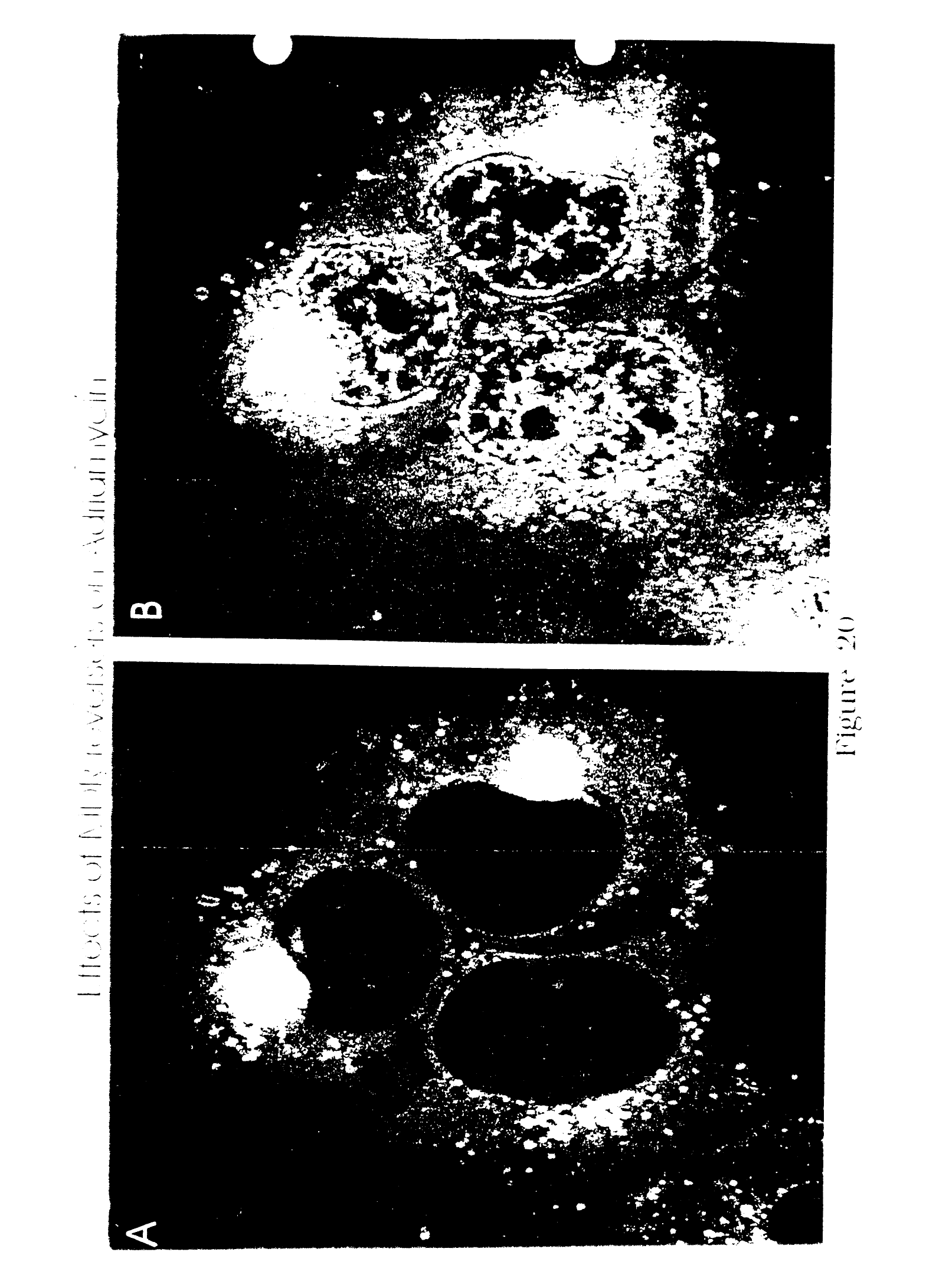

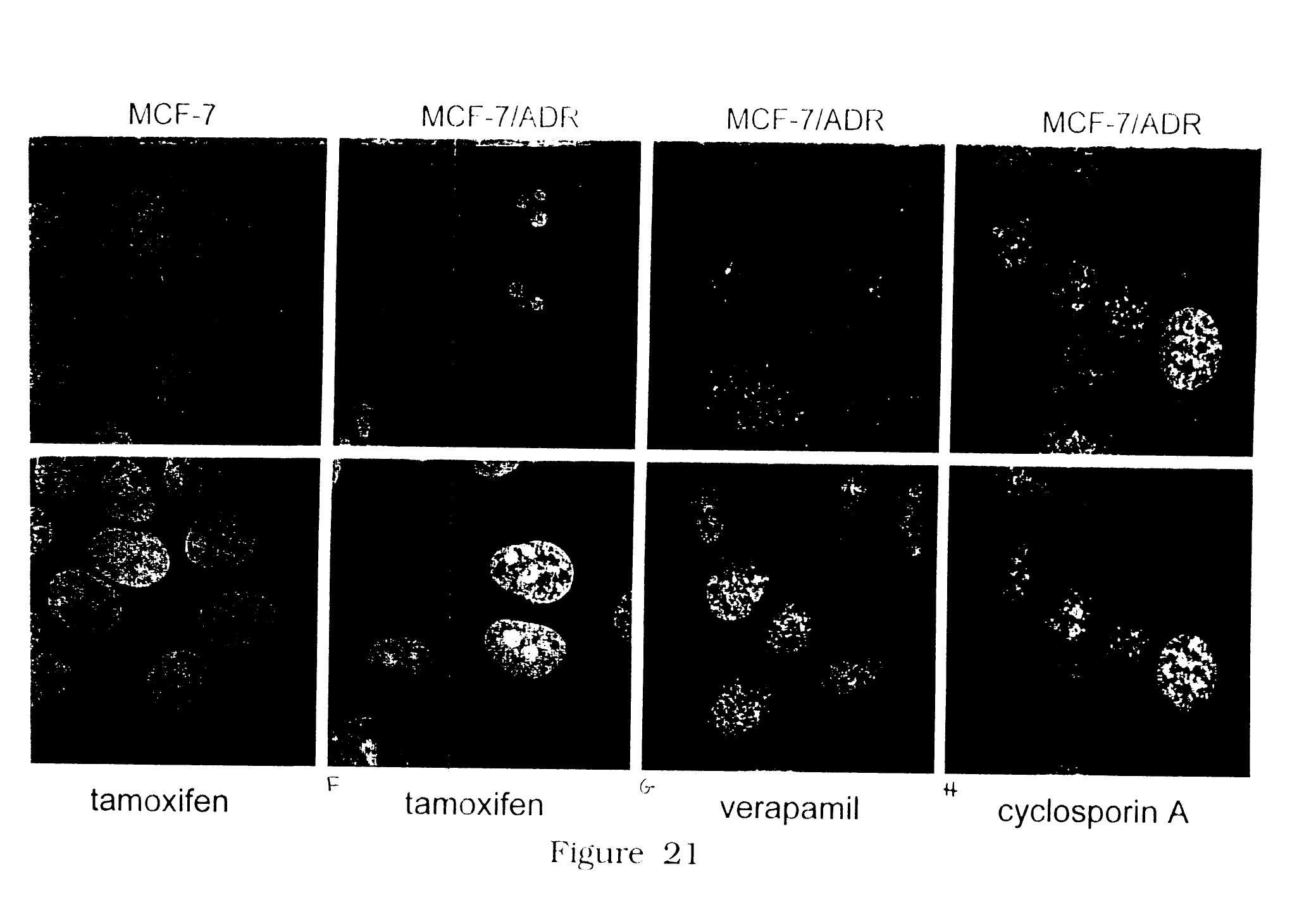

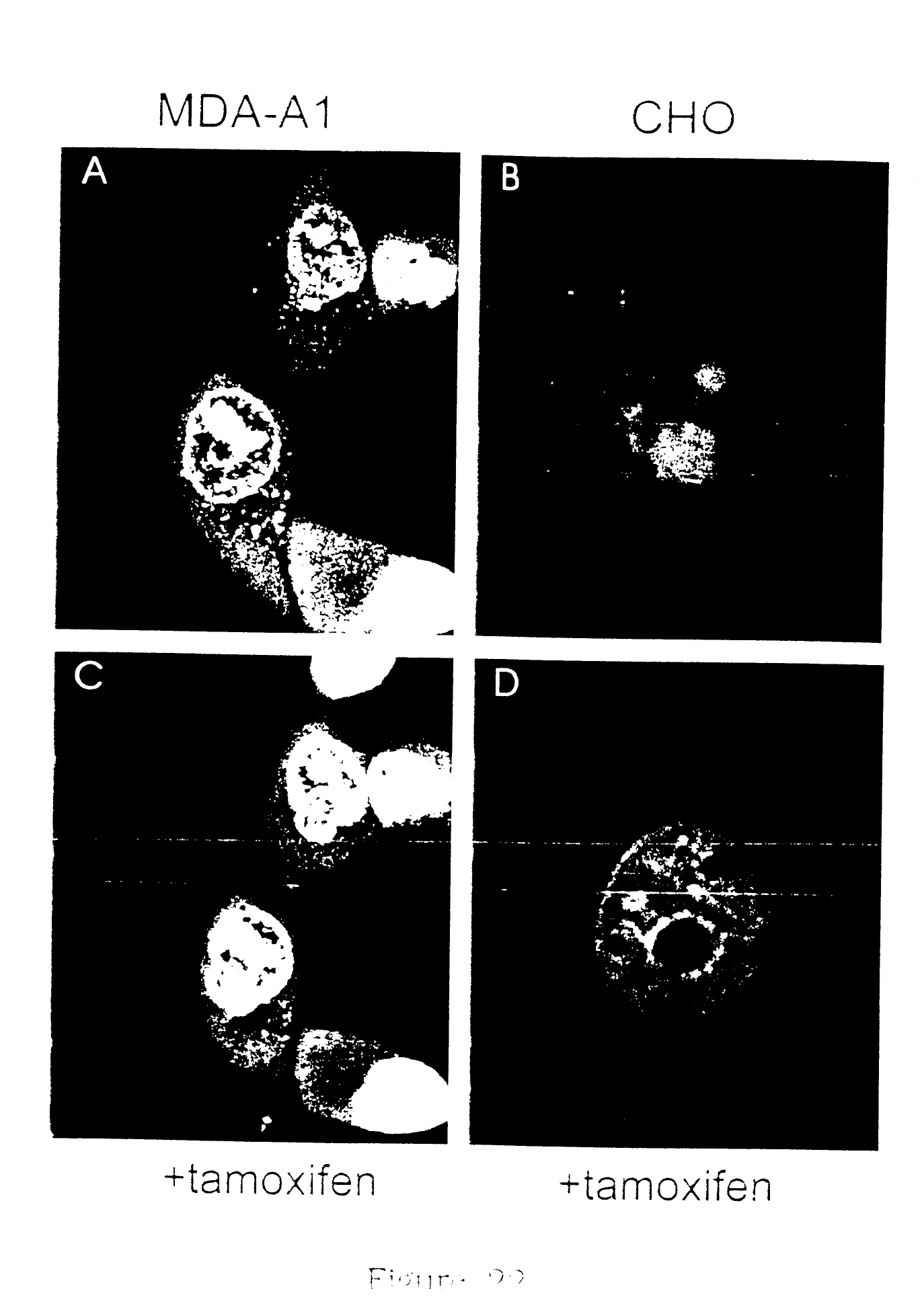

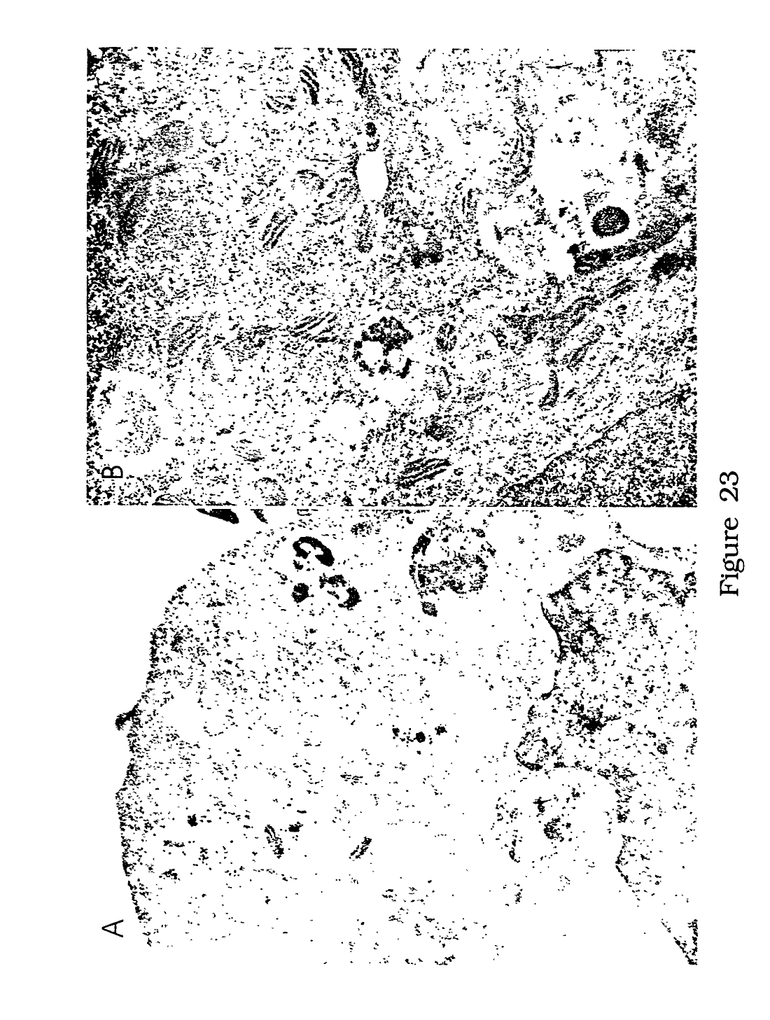

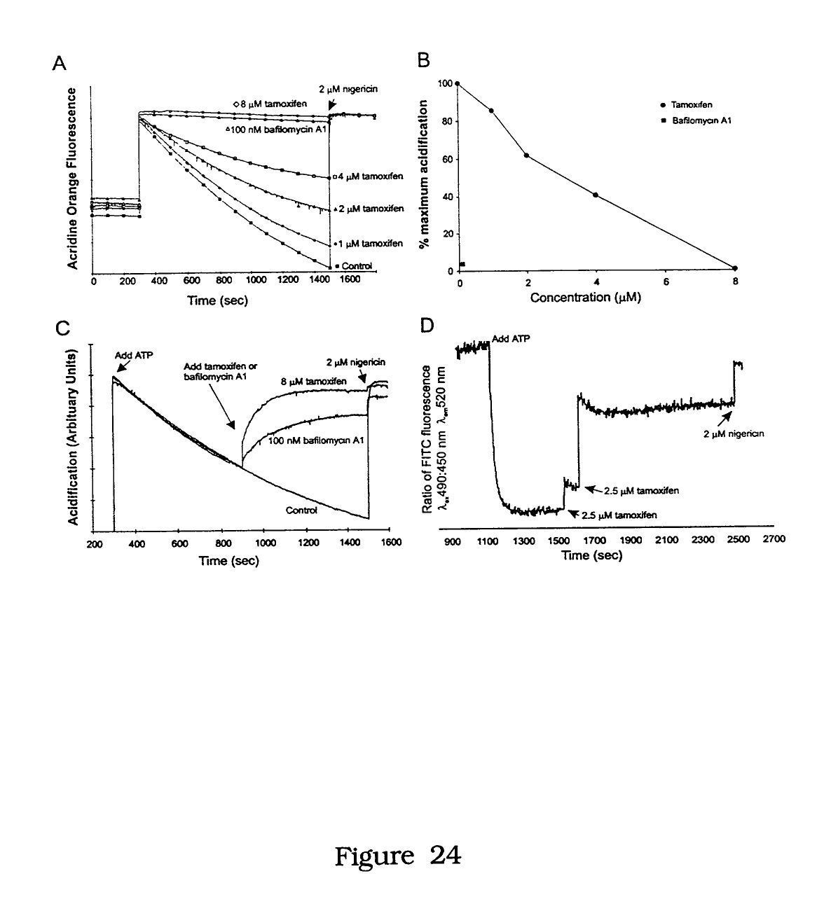

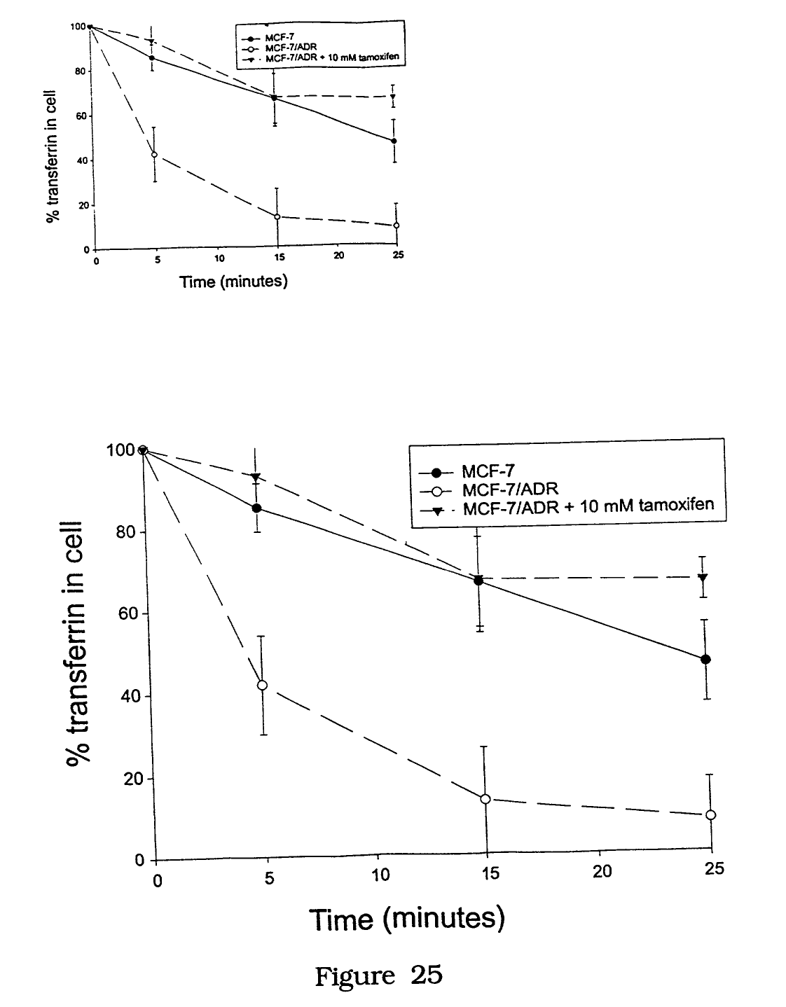

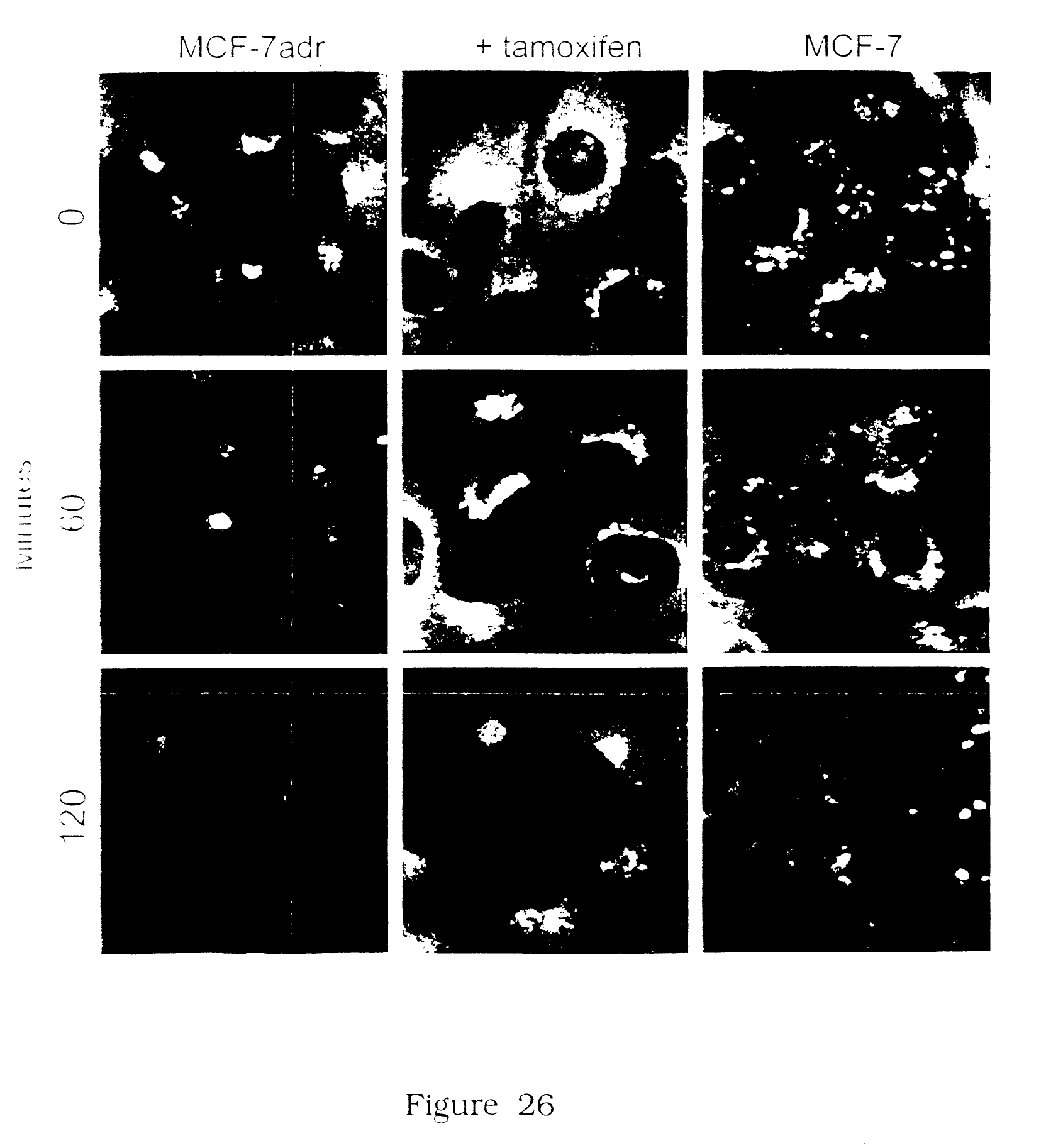

These measurements indicate that drug sensitive MCF-7 cells do not contain appropriately acidified late endosomes (pH 5.2-5.8), sorting endosomes (pH ˜6.0), endosomes (pH ˜6.0-6.3) [Mellman et al., Ann. Rev. Biochem., 55:663-700 (1986); Maxfield et al., Intracellular traficking of proteins (1991) and vanDeurs et al., International Review of Cytology, 117:131-177 (1989)], or secretory vesicles (pH ˜5.8) [Russell et al., J. Biol. Chem., 256:5950-5953 (1981)]. Thus, the MCF-7adr line more closely resembles normal, non-tumor cells, in this regard. The possibility was next examined as to whether the aberrant vesicular pH observed in MCF-7 cells is reflected in altered organization and activity of these compartments. Bodipy-ceramide (Bodipy-Ceramide) (Molecular Probes, Eugene, Oreg.), a fluorescent marker for the trans-Golgi network and secretory vesicles, showed a dispersed tubulo-vesicular distribution in MCF-7 cells (FIG. 7a) [Pagano et al., Journal of Cell Biology, 113:1267-1279 (1991)]. A large number of vesicular and cisternal structures appeared to be interconnected and possibly budding from a thin reticular network (enlarged image in FIG. 7b). In contrast, MCF-7adr cells labeled with Bodipy-Ceramide demonstrate asymmetrically localized pericentriolar structures characteristic of the trans-Golgi network (FIG. 7c). In addition, small labeled secretory vesicles (arrows) were found within the cytoplasm (FIG. 7d). Such vesicles were not easily detected in the MCF-7 cells (FIG. 7a). The organization and activity of the pericentriolar recycling compartment [Maxfield et al., in Intracellular Trafficking of Proteins, Steer & Hanover, ed., Cabridge U. Press, 157-182 (1991)] was examined with Bodipy-lactalbumin (Bodipy-LAC; Molecular Probes, Eugene, Oreg.), which was used as a fluorescent fluid phase marker. Steady state labeling of MCF-7 cells (FIG. 7e) showed uptake of fluorescent protein into peripheral vesicular compartments. No accumulation or aggregation of fluorescent vesicles was observed in association with a compartment within the pericentriolar region of the cytoplasm. In contrast, MCF-7adr cells (FIG. 7f) showed fluorescent vesicles associated with a labeled pericentriolar compartment.

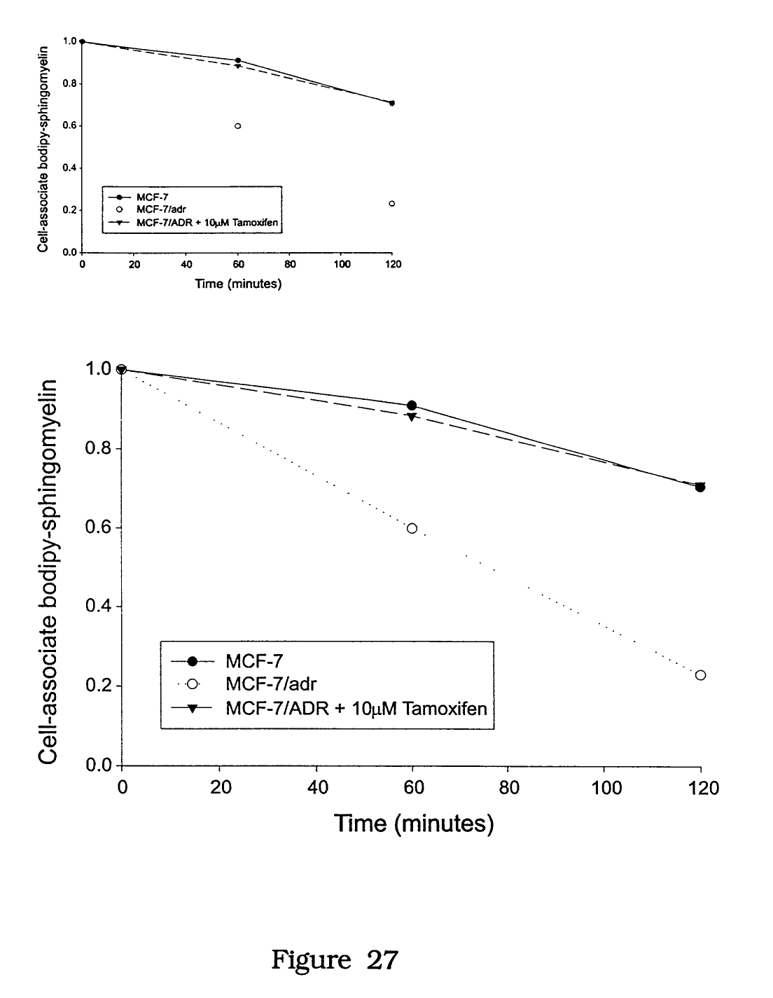

These observations suggested that the sensitivity of drug resistant cells to adriamycin might be overcome by chemically interfering with the capacity of these cells to acidify the vesicles that mediate internalization and secretion. As demonstrated in FIG. 8, addition of monensin to MCF-7adr cells at non-cytotoxic concentrations increased the drug sensitivity of the MCF-7adr cells to the level observed for drug-sensitive cells. The enhanced drug sensitivity of MCF-7adr cells treated with monensin was directly correlated to an alkalization of acidic vesicular compartments to pH values observed for drug sensitive MCF-7 cells (FIG. 6). Nigericin and amiloride functioned in a similar manner. These changes did not result from disruption of the Golgi since Brefeldin A (which breaks down the Golgi, but does not disrupt transport from the trans-Golgi or CURL to the cell surface [Lippincott-Schwartz et al., Cell, 67:601-616 (1991)] did not affect drug sensitivity.

Organelle acidification affects intracellular targeting, e.g. fusions of endosomes, secretory vesicles, and lysosomes; uncoupling of ligands from membrane receptors; processing and degradation of proteins; targeting of lysosomal enzymes; and glycosylation and packaging of secretory glycoproteins/glycolipids [Mellman et al., Biochem, 55:663-700 (1986); Maxfield et al., Intracellular trafficking of proteins, 157-182 (1991) and vandeurs et al., International Review of Cytology, 117:131-177 (1989)]. This communication demonstrates that the human breast cancer cell line (MCF-7) is defective in both the acidification of intracellular vesicles (FIG. 5) and the organization of the pericentriolar recycling compartment and the TGN (FIG. 7). These abnormalities appear repaired in the adriamycin resistant variant of this cell line (MCF-7adr). The role of a collapsed trans-vesicular pH gradient as the primary factor in producing drug sensitivity was strongly supported by results with ionophores. Monensin and nigericin enhanced the drug sensitivity of adriamycin resistant cells (MCF-7adr).

A mechanism for the pH shift observed in this study is suggested from work on parallel systems that show a similar shift in the lumenal pH of intracellular organelles. In endocytotic and secretory compartments an electrogenic ATPase coupled to a Cl⁻ conductance is responsible for maintaining the low pH [Barasch et al., Nature, 352:70-73 (1991); Van Dyke et al, J. Phsiol. Cell Physiol., 266:C81-C94 (1994) and Al-Awqati et al, Exp. Biol. 172:245-266 (1992)]. In cystic fibrosis, a decreased chloride conductance in the trans-Golgi compartment and recycling endosomes causes an alkaline shift of organellar pH [Barasch et al., Nature, 352:70-73 (1991) and Al-Awqati et al., Exp. Biol., 172:245-266 (1992)] In the absence of chloride, the lumenal pH shifts 0.4-0.6 units alkaline in multivesicular bodies (MVB), CURL vesicles, and receptor recycling compartments [Van Dyke et al., J/Physiol. Cell Physiol., 266:C81-C94 (1994)].

Analogously, an aberrant chloride conductance in the organelles of MCF-7 cells may cause the alkaline pH shift which is similar in magnitude to those observed in the previously cited examples (see Table 1). Likewise, the activation of a chloride conductance, or expression of a Cl⁻ conductance channel, in the MCF-7adr cells may then normalize the pH within acidic compartments. Adriamycin and a large number of drugs utilized for chemotherapy are weak bases which can be protonated and, thus, trapped in acidic compartments. Drug sensitivity of MCF-7 cells may be a consequence of an inability to protonate, sequester and then secrete these drugs (PSS model). Drug resistance is then an “ionic rehabilitation” of the normally acidic intracellular compartments through the expression of proteins (e.g. chloride channels or proton pumps) that compensate for this defect in acidification within tumor cells. One candidate protein for acidic rehabilitation is the p-glycoprotein which is expressed in many drug resistant cells, including the MCF-7adr. P-glycoprotein has been reported to function as a Cl⁻ channel [Valverde et al., Nature, 355:830-833 (1992)] or modify chloride conductance and is observed in the Golgi, vesicular and plasma membranes [Willingham et al., J. Histochem. Cytochem, 35:1451-1456 (1987) and Molinari et al., J. Cancer, 59:789-795 (1994)].

While multidrug resistance is likely to be the consequence of diverse mechanisms [Simon et al., Proc. Natl. Acad. Sci USA, 91:3497-3504 (1994)], the ability to reverse drug-resistance by drugs that alkalinize the pH in acidic compartments of the endosomal and secretory systems indicates that protonation, sequestration and secretion are the principle elements of the primary mechanism for drug resistance in the MCF-7 breast cancer line. Any manipulations that either affect acidification or transport through these organelles should affect drug-sensitivity. It is possible that the Golgi, particularly the secretory compartments, may normally play a role in protecting all cells from environmental toxins that are weak bases.

### EXAMPLE 3

### Tamoxifen Reverses Drug Resistance in MCF-7 Cells

Introduction

Treatment of tumors with chemotherapeutics can result in the appearance of cells displaying an acquired drug resistance to a spectrum of drugs. This resistance is termed multidrug resistance (MDR) [Simon et al., Proc. Natl. Acad. Sci USA, 91:3497-3504 (1994)]. It has been demonstrated that intracellular vesicular compartments do not acidify in drug sensitive MCF-7 human breast cancer cells but do acidify normally in drug resistant cells. This “ionic rehabilitation” of intravesicular pH in resistant cells could greatly facilitate the protonation, sequestration, and secretion of drugs through the normal activity of the recycling and secretory compartments. Acidification and efficient functioning of the tubulovesicular compartments comprising the efflux pathway is lost in the drug sensitive cells. As predicted from this hypothesis, agents which disrupt organelle acidification (e.g. monensin, nigericin and amiloride) are effective in reversing the resistance of MDR tumor cells [Simon et al., Proc. Natl. Acad. Sci USA, 91:3497-3504 (1994); Simon et al., Proc. Natl. Acad. Sci USA, 91:1128-1132 (1994)]. Here it is demonstrated that tamoxifen, an anti-estrogen which can reverse adriamycin resistance in vitro and in vivo [Simon et al., Proc. Natl. Acad. Sci USA, 91:3497-3504 (1994); Berman et al., Blood, 77:818 (1991) and Kirk et al., Biochem. Pharmacol., 48:277 (1994)], also disrupts the acidification and structure of the exocytotic compartments.

Results

Tamoxifen changes the intracellular distribution of chemotherapeutics in adriamycin resistant MCF-7 (MCF-7adr) as observed with confocal microscopy. The majority of adriamycin in MCF-7adr cells is sequestered within tubulovesicular compartments in pericentriolar region of the cell, a minimal level is found in the cytoplasm, and no fluorescence is observed in the nucleoplasm (FIG. 8, top row left). In contrast, in the drug sensitive (MCF-7) cells adriamycin is diffuse through the cell with an accumulation in the nucleus (FIG. 8, top row right). Treatment of MCF-7adr cells with tamoxifen (50 μM for 15 min.) shifts the adriamycin distribution to that observed in the MCF-7 cells (FIG. 8, top row middle). A similar redistribution of adriamycin to the nucleus has been reported following treatment with monensin and verapamil, two modifiers of drug resistance.

The pH of intracellular vesicular compartments was examined with acridine orange, a fluorescent reagent that accumulates in acidic compartments and undergoes a shift in fluorescence emission to longer wavelengths as a function of increased concentration [Barasch et al., Nature, 352:70-73 (1991)]. MCF-7adr cells show a pericentriolar localization of acridine orange staining, indicative of the uptake of acridine orange into acidic compartments (FIG. 8, 2nd row left) and no acidic compartments are observed in the MCF-7 cells (FIG. 8, 2nd row right). Treatment of the MCF-7adr cells with tamoxifen results in the loss of acridine orange staining within the pericentriolar region (FIG. 8, 2nd row middle) which was also seen following treatment with monensin and verapamil. These changes in acidification parallel the redistribution of adriamycin.

The effects of tamoxifen on the structure of the acidic exocytotic compartments was explored with fluorescent probes and confocal microscopy. To examine the structure and organization of the TGN in living cells, the fluorescent probe Bodipy-ceramide was exogenously added to cells in culture. In the Golgi, Bodipy-ceramide is converted to Bodipy-sphingomyelin which then migrates to the TGN. Accumulation of this metabolite in the TGN results in a long wavelength shift in its fluorescence emission (orange in FIG. 8, fourth row) and “red” labeling of the TGN and secretory vesicles. As observed in drug resistant cells (MCF-7-adr), the “red” TGN forms a crescent shaped structure within the pericentriolar region of the nucleus (FIG. 8, fourth row, left). This has been observed for Bodipy-ceramide labeling in a variety of cell types [Pagano et al., Journal of Cell Biology, 113:1267-1279 (1991)]. Drug sensitive MCF-7 cells show a pronounced disorganization of the TGN (FIG. 8, fourth row right) with an increase in tubulo-vesicular structures. These structures may represent defective formation or tethered secretory vesicles. A similarly disorganized TGN architecture has been observed in cells during mitosis and in cells treated with okadaic acid [Lucocq et al., J. Cel Sci., 103:875 (1992) and Horn et al., Biochem. J., 301:69 (1994)]. In all instances, a disrupted TGN architecture is shown to result in either no or defective secretion [Lucocq et al., J. Cell Sci., 103:875 (1992) and Horn et al., Biochem. J, 301:69 (1994)]. Treatment of the MCF-7adr cells with tamoxifen produces a similar fragmentation of TGN structure (FIG. 8, fourth row, middle).

Labeling of MCF-7 adr cells with bodipy-lactalbumin, a marker for the intracellular compartments involved in fluid phase endocytosis shows uptake of the dye-protein complex and localization within endosomes and elements of the pericentriolar recycling compartment (PRC) (FIG. 8, third row). Such localization was previously reported for other probes of the recycling pathway in a variety of cells [Koval et al., J. Cell. Biol., 108:2169 (1989) and Mayor et al., J. Cell Biol., 121:1257 (1993)]. In contrast, MCF-7 cells show only a very diffuse labeling with bodipy-lactalbumin (FIG. 8, 3rd row right). Treatment of MCF-7adr cells with tamoxifen disrupts the structure of the PRC to resemble the labeling in the MCF-7 cells (FIG. 8, 3rd row middle). Similar aberrant organization for the PRC has been described for endocytosis mutants [McGraw et al., J. Cell Physiol., 155:579 (1993)].

To examine the effect of tamoxifen on cytotoxicity, cell viability measurements were performed as described in FIG. 9. The addition of tamoxifen to MCF-7adr cells resulted in a significantly enhanced sensitivity to adriamycin.

An acidic pH within exocytotic compartments results in the protonation, sequestration and concentration of the drug within these vesicles. Rapid secretion of drug is then achieved through the normal activity of the recycling/secretory pathway. Abnormal alkalization of these acidic exocytotic compartments in adriamycin sensitive cells results in the accumulation of drug within the nucleus and cytoplasm leading to cytotoxicity. The ability of tamoxifen to block, or reverse, acidification of these organelles provides an effective means to inhibit drug accumulation within exocytotic compartments and in this manner increase cytosolic and nucleoplasmic concentration of cytotoxic drugs. Tamoxifen has been demonstrated to block ATP-dependent chloride channels [Zhang et al., J. Clin. Invest., 94:1690 and Ehring et al., J. Gen. Physiol., 104:1129 (1994)] and Cl⁻ is an important counterion for allowing the establishment of proton gradients across the membranes of endosomal and secretory vesicles. The observation that the multi-drug resistance protein Pgp is heavily localized within the Golgi in MCF-7adr cells [Willingham et al., J. Histochem. Cytochem, 35:1451-1456 (1987) and Molinari et al., Int. J. Cancer, 59:789-795 (1994)] suggest that it may be responsible for the “ionic rehabilitation” of the secretory compartments in MCF-7 cells and the resultant drug resistance phenotype.

### EXAMPLE 4

### Defective Acidification in Human Breast Tumor Cells and Implications for Chemotherapy

| | Abbreviations |
|---|---|
| DMEM: | Dulbecco's modified eagle medium |
| MDR: | multidrug resistance |
| MRP: | multidrug resistance associated protein |
| Pgp: | P-glycoprotein |
| PSS: | Protonation, sequestration and secretion |
| SNARF: | seminaphthorhodafluor |
| TGN: | trans-Golgi network |

Introduction

Successful chemotherapy requires that tumor cells be more sensitive to chemotherapeutic agents than normal cells of the body. However, the main impediment to such treatment of cancer is the development of resistance by the tumor not only to the drugs administered but also to a host of other structurally and mechanistically diverse drugs to which the tumor has not been exposed [Biedler and Riehm, Cancer Res., 30:1174-1184 (1970)]. This phenomenon has been termed multidrug resistance (MDR).

Research during the past 20 years has discovered many genetic differences between drug-resistant and drug-sensitive tumor cells [Simon and Schindler, Proc. Natl. Acad. Sci. USA, 91:3497-3504 (1994b)]. These include changes in the type and amount of cellular lipids and in the expression of proteins including p-glycoprotein (Pgp) [Ling, Can. J. Genet. Cytol., 17:503-515 (1975); Gottesman and Pastan, Annu. Rev. Biochem., 62:385-427 (1993)], MDR associated protein (MNP) [Cole et al., Science, 258:1650-1654 (1992); Slovak et al., Cancer Res., 53:3221-3225 (1993)], glutathione S-transferase [Harris and Hochhauser, Acta Oncol., 31:205-213 (1992); Efferth and Volm, Cancer Lett., 70:197-202 (1993); Volm and Mattern, Onkologie, 16:189-194 (1993); De la Torre et al., Anticancer Res., 13:1425-1430 (1993); Ripple et al., J. Urol., 150:209-214 (1993)], protein kinase C [Posada et al., Cancer Commun., 1:285-292 (1989); Ahmad et al., Mol. Pharmacol., 42:1004-1009 (1992); Chaudhary and Roninson, Oncol. Res., 4:281-290 (1992); Efferth and Volm, Anticancer Res., 12:2209-2212 (1992); Lelong et al., Biochemistry, 33:8921-8929 (1994); Hardy et al., EMBO Journal, 14:68-75 (1995)], DNA topoisomerase II [Friche et al., Cancer Res., 51:4213-4218 (1991); Beck, Cancer Treat. Rev., 17 Suppl A:11-20 (1990); Beck, J. Natl. Cancer Inst., 81:1683-1685 (1989)], and proton ATPase [Ma and Center, Biochemical and Biophysical Research Communications, 182:675-681 (1992)]. In addition to these genetic differences, drug-resistant and sensitive cells have many phenotypic and cell biological differences. Chief among them are differences in their pH profile for the cell cytoplasm and intracellular compartments [Keizer and Joenje, J. Natl. Cancer Inst., 81:706-709 (1989); Thiebaut et al., Journal of Histochemistry and Cytocheinistry, 38:685-690 (1990); Roepe et al., Biochemistry, 33:11008-11015 (1994); Simon et al., Proc. Natl. Acad. Sci. USA, 91:1128-1132 (1994a)]. The cytoplasmic pH of drug-sensitive cells has been consistently found to be more acidic than that of drug-resistant cells. Further, drug-sensitive cells lack many acidified intracellular organelles seen in drug-resistant and non-transformed cells [Schindler et al., Biochemistry, 35:2811-2817 (1996)]. Finally, there are dramatic differences in the intracellular distribution of chemotherapeutic drugs: In drug-sensitive cells, chemotherapeutic drugs are diffuse throughout the cytoplasm and nucleus. In contrast, in drug-resistant cells chemotherapeutics accumulate only within discrete cytoplasmic organelles; almost none is detectable in the nucleus [Willingham et al., Cancer Res., 46:5941-5946 (1986); Hindenburg et al., Cancer Res., 49:4607-4614 (1989); Gervasoni, Jr., et al., Cancer Res., 51:4955-4963 (1991); Lankelma et al., Biochim. Biophys. Acta Mol. Cell Res., 1093:147-152 (1991); Weaver et al., Exp. Cell Res., 196:323-329 (1991); Jaffrézou et al., Cancer Res., 52:6440-6446 (1992); Coley et al., Br. J. Cancer, 67:1316-1323 (1993); Rutherford and Willingham, Journal of Histochemistry & Cytochemistry, 41:1573-1577 (1993)].

Many chemotherapeutic drugs, such as the anthracyclines and vinca alkaloids, are weak bases with pKa values between 7.4 and 8.4 [Burns, Analytical Profiles of Drug Substances, 1:463-480 (1972); Beijnen, Analytical Profiles of Drug Substances, 17:221-258 (1988)]. They are membrane permeable in their neutral form and membrane impermeable when protonated. When these drugs diffuse into acidified liposomes or acidified red blood cell ghosts, they become protonated, thus membrane impermeable, and sequestered [Dalmark and Storm, J. Gen. Physiol., 78: 349-364 (1981); Dalmark and Hoffmann, Scand. J. Clin. Lab. Invest., 43:241-248 (1983); Mayer et al., Biochim. Biophys. Acta, 857:123-126 (1986); Beck, Biochem. Pharm., 36:2879-2887 (1987); Burke et al., Mol. Pharmacol., 31:552-556 (1987)]. Likewise, upon entering any of the acidic compartments of the cell (such as the lysosome, recycling endosomes, TGN or secretory vesicles) they should become protonated, and sequestered within these compartments. Based on these observations, the protonation, sequestration, and secretion (PSS) model has been proposed to account for the relative sensitivity of tumor cells to weak base chemotherapeutics and the resistance of MDR cells [Example 5]. The PSS hypothesis postulates that acidified organelles (in MDR and non-transformed cells) protonate chemotherapeutic drugs, thereby sequestering them from the nucleoplasm and cytosol (the aqueous phase of the cytoplasmic compartment). The drugs are subsequently secreted from the cell through the normal pathways of vesicular traffic and secretion. The model proposes that the enhanced sensitivity of tumor cells to chemotherapeutics is a consequence of a reduced acidification within these organelles and, thus, a reduced ability to sequester the drugs away from the cytosol and nucleoplasm. The PSS hypothesis makes the following four predictions: (1) chemotherapeutics should accumulate within the acidic secretory organelles of drug-resistant cells; (2) there should be a significant quantitative difference between drug-sensitive and MDR tumor cells in either the organellar acidification or transport; (3) agents that disrupt organellar acidification should reverse drug-resistance, and (4) agents that reverse drug resistance should either block acidification or block secretion from acidified organelles.

Materials and Methods

Materials: Bodipy-transferrin, Lysosensor Blue DND-167, FITC-transferrin, seminaphthorhodafluor (SNARF)-dextran, NBD-ceramide, and FITC-dextran were from Molecular Probes (Eugene, Oreg.). Adriamycin was from Calbiochem (San Diego, Calif.). Concanomycin A was from Fluka (Milwaukee, Wis.). Bovine insulin and L-glutamine from Gibco (Gaithersburg, Md.) and FBS was from Gemini Bio-Products (Calabasas, Calif.). The anti LAMP-1 serum was from the Developmental Hybridoma Bank (Johns Hopkins University, Baltimore, Md.) and goat anti mouse secondary antibody Fab fragments conjugated to phycoerythrin were from Jackson Immunochemicals (West Grove, Pa.). All other reagents were from Sigma (St. Louis, Mo.).

Tissue culture: MCF-7 and MCF-7/ADR cells were obtained from Dr. William Wells of the Department of Biochemistry at Michigan State University. They were maintained in Modified Eagle's medium with phenol red, Bovine insulin 10 μg/mL and L-glutamine and 10% FBS in a humidified incubator at 37° C. and 5% pCO₂ (Forma Scientific, OH). In addition, the MCF-7/ADR cells were continuously maintained in 0.8 μM Adriamycin. The MCF-10F cells were obtained from the American Type Culture Collection (Rockville, Md.)

Cell Imaging: Unless otherwise stated, cells were incubated in Dulbecco's modified eagle medium (DMEM) without phenol red or serum and with 20 mM HEPES pH 7.3 and the fluorescent dye at 37° C. in Labtek (Naperville, Ill.) coverglass chambers for imaging.

Confocal microscopy: Unless specifically stated, all imaging measurements were performed on a Meridian confocal microscope, equipped with an Argon laser (Meridian Inc, Okemos Mich.). Cells were visualized with an Olympus 60×/1.4 NA oil objective, and the data were collected with two R3896 photomultiplier tubes (Hamamatsu Photonics, Hamamatsu City, Japan). Cells were kept in a 37° C. chamber with superfused humidified air containing 5% pCO₂.

Epifluorescence microscopy: A Nikon Diaphot fluorescence microscope was used for the pH measurements within the lumens of recycling endosomes. The microscope was equipped with a 100 W Hg lamp and Uniblitz shutter (Vincent and Associates, Rochester N.Y.). The shuttering of the light source was controlled with a computer. A filter holder was manufactured to hold 450 nm and 490 nm excitation filters. The data were collected on a Hamamatsu 4910 intensified charged coupled device (Hamamatsu Photonics). Cells were kept at 37° C. with a Bioptiks objective heater (Butler, Pa.) and superfused with humidified air at 37° C. with 5% CO₂.

Adriamycin Labeling: Adriamycin is a small heterocyclic amine (molecular wt. 580 Dalton) with a pK of 8.3 that can diffuse across membranes in the uncharged form. Adriamycin can be excited between 350 nm-550 nm and emits between 400 nm-700 nm. Cells were incubated with Adriamycin (10 μM) for 30 minutes at 37° C. and then visualized with the confocal microscope using 488 nm line of the argon laser.

Acridine orange labeling: Cells were incubated with acridine orange (6 μM) for 15 minutes and then visualized on the confocal microscope with the 488 nm line of the argon laser. The green and red emissions were collected using two photomultiplier tubes. Emission light was first passed through a 560 nM short-pass dichroic mirror. Green emission was next passed through a 530 nm/30 nm bandpass filter and collected by the on-axis photomultiplier tube. Red emission was passed through a 610 nm longpass filter and collected by the 90° side photomultiplier tube.

Lysosome labeling: Cells were incubated with Lysosensor Blue DND 167 [Haugland, in Molecular Probes, Hand Book of Fluorescent Probes and Research Chemicals, 6th ed., Eugene, Oreg., p.278 (1996)] (2 μM, 1 mM stock in water) for 60 minutes, and then visualized on the confocal microscope using the 353 nm line of the argon laser. In some experiments the cells were subsequently washed and then incubated with Adriamycin (10 μM) for 30 minutes.

TGN labeling with NBD-ceramide: Cells growing on Labtek coverslip chambers were incubated in DMEM/20 mM HEPES pH 7.3 containing of NBD-Ceramide (5 μM) at 4° C. for 10 minutes [Pagano et al., J. Cell Biol., 113:1267-1279 (1991)]. They were then washed twice with DMEM/20 mM Hepes pH 7.3/10% FBS and incubated at 37° C. for 30 minutes and placed on the confocal microscope for observation using the 488 nm line of the Argon laser.

Bodipy-transferrin labeling of the recycling endosome compartment: BODIPY-transferrin was used to label the recycling endosome compartment for structural studies. Transferrin is endocytosed by specific transferrin receptors on the surface of the cell. After endocytosis the transferrin is transported through the endosomes and then recycled back to the surface. The transferrin receptor is not transported to the lysosomes, so probes that are conjugated to transferrin can be used to selectively monitor the recycling endocytic compartments [Fuller and Simons, J. Cell Biol., 103:1767-1779 (1986); Ghosh and Maxfield, J. Cell Biol., 128:549-561 (1995)]. The endocytic pathway is known to undergo acidification [Schmid et al., J. Cell Biol., 108:1291-1300 (1989)]. Thus, the fluorophore BODIPY was used as a probe on transferrin since its fluorescence is not very sensitive to pH. The cells were loaded with 150 μg/ml of BODIPY transferrin in DMEM/20 mM HEPES pH 7.3 for 25 minutes in a humidified incubator at 37° C. and 5% CO₂ [Ghosh and Maxfield, J. Cell Biol., 128:549-561 (1995)]. It was determined from competition binding studies with unlabelled transferrin that this amount of BODIPY transferrin specifically labels the recycling endocytic compartment in MCF-7 and MCF-7/ADR cells while giving a detectable signal under epifluorescence. The cells were washed rapidly three times with DMEM/HEPES and three times with Hanks Balanced salt solution (HBSS) containing 20 mM HEPES, pH 7.3. In some experiments the cells were subsequently incubated at 37° C. for 30 minutes to allow the transferrin to recycle to the surface and then the cells were incubated with Adriamycin (10 μM) at 37° C. for 30 minutes.

pH measurements: The pH sensitive fluorophores, FITC and SNARF, were used to measure the pH within endosomes and the cytosol, respectively. Lysosensor Blue DND-167 is a third fluorophore that was used as an independent probe specifically for calibration of the pH within the lumenal compartment of lysosomes. Both FITC and SNARF are ratiometric dyes. The emission intensity of FITC at 530 nm increases with increasing pH with excitation at 490 nm. However, it is unaffected by pH when the fluorophore is excited at 450 nm. Therefore, by taking the ratio of the emission intensities at the two excitation wavelengths, one can obtain a pH value independent of FITC concentration in a particular compartment. To convert the ratios to pH values, the cells were calibrated using monensin and nigericin with buffers of known pH (see below). FITC is most useful for measurement of pH values from 5.0 to 7.0.

SNARF, when excited at 514 nm, emits at two wavelengths: 570 nm and 630 nm. The protonated fluorophore emits at 570 nm and the neutral fluorophore emits at 630 nm. Again, the ratio of the two emissions corresponds to a pH value that is independent of the concentration of the dye in that compartment. SNARF can be reliably calibrated over the pH range of 6.2 to 9.0. The fluorescence of Lysosensor Blue DND-167 is dependent on pH. Lysosensor Blue has a functional group that, when deprotonated, leads to a loss of fluorescence of the molecule. The pK of this group is 5.1 Therefore at pH<5.1, a greater percent of the dye will be protonated and will be fluorescent. There is little fluorescence above pH 5.8.

At the end of each experiment, the fluorescence emission of each dye was calibrated with solutions of known pH. For pH calibration of endosomes, the cells were incubated in solutions of 150 mM NaCl, 20 mM HEPES,5 mM KCl, 1 mM MgSO4 buffered at pH's 5, 6, 6.5, 7, containing monensin (20 μM) and nigericin (10 μM) for 5 minutes before recording the fluorescence . For the pH calibrations of cytosol and nucleoplasm, the cells were incubated in solutions of 140 mM KCl, 10 mM MOPS, 5 mM MgSO4, 1 mM CaCl2 buffered at pH's 6, 7, 7,5 containing nigericin (20 μM).

Recycling compartment pH measurement: The probe FITC bound to transferrin was used to selectively probe the pH of the endocytic compartment. FITC [Schmid et al., J. Cell Biol., 108:1291-1300(1989); Ghosh and Maxfield, J. Cell Biol., 128:549-561 (1995)]. The cells were loaded with FITC-transferrin using the same protocol used to label the endocytic compartment with BODIPY-transferrin. The fluorescence was recorded in Hanks buffered salt solution (HBSS) buffered with 20 mM HEPES at pH 7.3. The pH was calibrated from the FITC fluorescence as described above.

Lysosome pH: To measure the pH within the lysosomes, the cells were incubated with 5 mg/mL of FITC dextran 10 kD for 30 minutes [Yamashiro and Maxfield, J. Cell Biol., 105:2723-2733 (1987)]. Then the cells were washed 4 times in DMEM with 20 mM HEPES pH 7.3, and incubated in this medium for 90 minutes. They were then visualized on a Nikon Diaphot equipped with FITC excitation filters (see above). The pH was calibrated from the FITC fluorescence as described above. Alternatively the cells were incubated with Lysosensor Blue as described above.

pH of the Cytoplasm and Nucleoplasm: The pH within the cytoplasm and nucleoplasm was selectively probed by loading these compartments with SNARF conjugated to dextrans using a procedure referred to as “scrape loading” [McNeil et al., J. Cell Biol., 98:1556-1564 (1984), hereby incorporated by reference in its entirety]. Briefly, the cells were plated on polystyrene plates at 50% confluency 24-36 hours before loading with dextrans. The medium was aspirated off the dishes, and the cells were covered with 50 μL of the SNARF dextran at 10 mg/ml concentration. The cells were then quickly scraped off the polystyrene with a rubber scraper and placed in pre-chilled tubes containing 1 mL of media without serum. The cells were harvested by spinning at a force of 100 g for 5 minutes. The medium was aspirated and replaced again with prechilled media without serum and the cells harvested again by spinning. Finally the medium was aspirated and replaced with one containing serum and the cells were plated on poly-lysine coated glass cover-slip chambers. The cytosolic pH was selectively probed by loading the cytosol with a 70 kD SNARF-conjugated dextran. This dextran is too large to enter into organelles or the nucleus. The nucleoplasmic pH was probed by loading the cytosol with SNARF conjugated to a 10 kD dextran. This is too large to cross cellular membranes, but can enter the nucleoplasm by diffusion across the nuclear pores. Confocal fluorescence microscopy was used to prepare optical sections through the cell. The fluorescence intensity of the nucleoplasm and cytoplasm could then be quantified. The fluorescence from the SNARF-conjugated dextrans was recorded 24-36 hours after scrape loading. The pH was calibrated from the fluorescence as described above.

Immunofluorescence: LAMP-1: For immunolocalization of lysosomes anti-LAMP-1 serum was employed as described by Hoock et al. [J. Cell Biol., 136:1059-1070(1997)]. Cells were fixed with 2% paraformaldehyde in 50 mM phosphate buffer pH 7.8 containing lysine (9 mg/mL) for 2 hours. They were then permeabilized with 0.01% saponin for 5 minutes. Anti-LAMP-1 sera was used undiluted for 30 minutes at room temperature. Cells were washed extensively with PBS and then incubated for 15 minutes with goat anti mouse secondary antibody Fab fragments conjugated to phycoerythrin at 1:150 dilution at room temperature. Cells were washed in PBS and visualized with the confocal microscope using excitation wavelength 488 nm.

Results

Adriamycin distribution in drug-resistant MCF-7/ADR and drug-sensitive MCF-7 cells: The protonation, sequestration and secretion hypothesis disclosed herein predicts that weak base chemotherapeutics should accumulate in the acidic secretory organelles of drug-resistant cells. Adriamycin was chosen as the model chemotherapeutic drug to characterize the subcellular distribution of these agents in drug-sensitive and drug-resistant tumor cells because its natural fluorescence allows it to be tracked visually and it is widely administered in the treatment of many different types of cancers. MCF-7 and MCF-7/ADR cells were employed as a pair of drug-sensitive and drug-resistant cell lines respectively. They are human breast carcinoma cells that are used as an in vitro model system for breast cancer.

The drug-resistant MCF-7/ADR cell line is derived from the MCF-7 cell line by selection in the chemotherapeutic Adriamycin [Vickers et al., Molecular Endocrinology, 2:886-892 (1988)]. MCF-7/ADR cells are also cross-resistant to a number of other chemotherapeutic drugs including vincristine, vinblastine and colchicine.

In the drug-sensitive MCF-7 cells, Adriamycin fluorescence was seen throughout the cytoplasm and nucleoplasm (FIG. 10b). Some localized increased fluorescence of Adriamycin can be seen in both the cytoplasm and nucleoplasm. One of the primary targets for Adriamycin is in the nucleus where it binds to DNA and inhibits the DNA metabolic enzyme topoisomerase II, thereby blocking DNA replication and transcription [Di Marco et al., Antiboit. Chemother., 23:12-20 (1978); Zunino et al., Biochim. Biophys. Acta, 476:38-46 (1977); Harris and Hochhauser, Acta Oncol., 31:205-213 (1992)]. In contrast, in the drug-resistant MCF-7/ADR cells, there was little Adriamycin fluorescence in the nucleoplasm and an apparent reduction of fluorescence in the cytoplasm (FIG. 10a). Instead, the drug was found to be accumulated in a perinuclear region and within punctate compartments throughout the cytoplasm.

Adriamycin co-localizes with the acidic compartments of Lysosoines, recycling endosomes and the TGN, in MCF-7/ADR cells. Since Adriamycin is a weak base, it is expected to accumulate inside acidic compartments. To determine if it accumulated in the lysosomes, the most acidified cellular compartment, cells were sequentially labeled with Adriamycin and Lysosensor Blue DND167. Lysosensor blue is a membrane permeable pH probe whose fluorescence emission is significantly reduced at pH>5.8 (see above). Thus, it selectively fluoresces only in the most highly acidic compartments of living cells such as lysosomes. Discrete punctate fluorescence in the cytoplasm was observed in cells incubated with Lysosensor Blue (FIG. 11b). These lysosomes appeared to be somewhat evenly distributed through the cytoplasm. The same cells were then incubated with Adriamycin and excited at 488 nm and the emission collected above 550 nm (FIG. 11c). Lysosensor Blue is not excited at 488 nm, therefore it did not interfere with the Adriamycin signal. Most lysosomes (arrows in FIG. 11b) were labeled with Adriamycin (arrows in FIG. 11c). In addition to the lysosomal compartment there was an additional cytoplasmic region adjacent to the nucleus that was heavily labeled with Adriamycin (FIG. 11c).

In many cell types the TGN and the recycling endosome compartment are found adjacent to the nucleus. These two compartments are also known to be acidic [Glickman et al., J. Cell Biol., 97:1303-1308 (1983); Mellman et al., Annu. Rev. Biochem., 55:663-700 (1986); Kim et al., J. Cell Biol., 134:1387-1399 (1996)]. Therefore, specific fluorescent probes were used to determine if the perinuclear Adriamycin accumulation in the MCF-7/ADR cells co-localized with these compartments. Due to the broad excitation and emission spectrum of Adriamycin, it was not possible to simultaneously label a cell with Adriamycin and available fluorescent markers for endosomes and the TGN. Therefore, co-localization was done by sequential labeling with Adriamycin and the organelle markers.

To examine whether Adriamycin co-localized with the recycling endosome compartment, cells were first labeled with BODIPY transferrin by receptor mediated endocytosis. In MCF-7/ADR cells, transferrin labeling revealed that the recycling endosomal compartment has a perinuclear localization (FIG. 11e). The transferrin label was subsequently chased out (half time=5 minutes) with unlabeled transferrin. Next the cells were incubated with Adriamycin for 20 minutes. In each cell the region that labeled with BODIPY transferrin was also labeled with Adriamycin (FIG. 11f). Thus, the Adriamycin accumulation co-localized with the recycling endosomes.

In some cell types the TGN is in close proximity with the recycling endosome compartment [Presley et al., J. Cell Biol., 122:1231-1241 (1993); McGraw et al., J. Cell PHysiol., 155:579-594 (1993)]. To test if Adriamycin co-localized with the TGN in the MCF-7/ADR cells, the cells were sequentially labeled with Adriamycin and NBD-ceramide, a vital stain for the Golgi [Lipsky and Pagano, Science, 228:745-747 (1985)]. A field of cells was labeled with Adriamycin (10 μM) (FIG. 11i). The cells were then washed with Adriamycin-free media until they no longer showed any Adriamycin labeling. Next, the cells were labeled with NBD-ceramide (FIG. 11h). Again, the region labeled with NBD-ceramide was also labeled with Adriamycin. Thus, Adriamycin accumulation is co-localized with the acidic organelles of the drug-resistant MCF-7/ADR cells: the recycling endosome compartment, the TGN and the lysosomes.

Location of the lysosomes, recycling endosome compartment, and the TGN in drug-sensitive MCF-7 cells. There was little organellar labeling of Adriamycin in drug-sensitive MCF-7 cells (FIG. 10b). Therefore the presence and distribution of each of these organelles in these cells was tested to determine whether the organelles were missing, or whether they were present but failing to accumulate chemotherapeutics. The distribution of the recycling endosome compartment was probed with BODIPY-transferrin and the TGN was probed with NBD-ceramide [Lipsky and Pagano, Science, 228:745-747 (1985)]. In the drug-sensitive MCF-7 cells both the recycling endosome compartment (FIG. 11d) and the TGN (FIG. 11g) were distributed throughout the cytoplasm. Their distribution was very different from the MCF-7/ADR cells where both compartments were distinctly perinuclear in location and polarized to one side of the nucleus. Furthermore, unlike the MCF-7/ADR cells, in the MCF-7 cells, Adriamycin was not concentrated in any of these compartments (FIG. 10b). Instead the Adriamycin distribution was diffusely cytoplasmic and nucleoplasmic.

The distribution of lysosomes in MCF-7 and MCF-7/ADR cells was compared using immunofluorescence for the lysosomal integral membrane protein, LAMP-1 [Chen and Schnell, J. Biol. Chem., 272:6614-6620 (1997)]. LAMP-1 was chosen as an indicator rather than Lysosensor Blue because Lysosensor Blue requires acidified lysosomes for labeling. Both MCF-7/ADR cells (FIG. 12a) and MCF-7 cells (FIG. 12b) had punctate LAMP-1 labeled compartments in the cytoplasm. There was little qualitative or quantitative difference in the distribution of LAMP-1 labeling between the two cell types. In some MCF-7 and MCF-7/ADR cells there was also a perinuclear staining for LAMP-1 which could be indicative of lysosomes or TGN since this membrane protein is sorted to the lysosomes from the TGN. Together the results demonstrate that the recycling endosomes, TGN and lysosomes are present in the MCF-7 cells, but they do not accumulate Adriamycin.

Subcellular pH profiles of MCF-7 and MCF-7/ADR cells: Many of the chemotherapeutic drugs such as Adriamycin, vincristine, vinblastine, daunomycin and mitoxantrone are heterocyclic amines (see FIG. 13) [Vigevani and Williamson, Analytical Profiles of Drug Substances, 9:245-274 (1980); Burns, Analytical Profiles of Drug Substances, 1:463-480 (1972); Beijnen, Analytical Profile of Drug Substances, 17:221-258 (1988)]. With pK values at, or just above physiological pH, they are weak bases and are membrane permeable only in the non-charged form. This has been tested empirically in single membrane model systems of liposomes [Mayer et al., Biochim. Biophys. Acta, 1025:143-151 (1990)] and anucleate red blood cells [Dalmark and Storm, J. Gen. Physiol., 78:349-364 (1981); Dalmark and Hoffmann, Scand. J. Clin. Lab. Invest., 43:241-248 (1983)]. In liposomes in which the lumenal compartments are more acidic relative to the external medium, there is a net lumenal accumulation of Adriamycin. The magnitude of this pH dependent lumenal accumulation closely approximates theoretical calculations. For example, Adriamycin with pKa of 8.3 accumulates approximately 100 fold in a liposome with a lumenal pH of 6 and an external pH of 8 [Mayer et al., Biochim. Biophys. Acta, 1025:143-151 (1990)]. As shown above (FIGS. 11b, c, e, f, h, i), Adriamycin accumulation co-localized with each of the acidic organelles of the cell. However, Adriamycin did not accumulate within these same organelles in the drug-sensitive MCF-7 cells. Therefore, the pH was examined in the cytosol and organelles of MCF-7 and MCF-7/ADR cells to determine whether the failure to concentrate Adriamycin in the organelles of MCF-7 cells was the consequence of a failure to acidify.

Qualitative pH profile monitored with Acridine Orange. Acridine orange is a fluorescent weak base that is frequently used as a probe for acidification of organelles [Barasch et al., J. Cell Biol., 107:2137-2147 (1988); Cain and Murphy, J. Cell Biol., 106:269-277 (1988); Yoshimori et al., J. Biol. Chem., 266:17707-17712 (1991); Zelenin, In Flourescent and luminescent probes for biological activity, W. T. Mason, ed., San Diego, Calif.: Academic Press, pp.83-99 (1993)]. When accumulated in high concentrations in acidic compartments, it undergoes a spectral shift from green to red [Zelenin, 1993, supra]. MCF-7 and MCF-7/ADR cells were incubated with acridine orange. In drug-resistant MCF-7/ADR cells, there was a red-orange fluorescence in discrete cytoplasmic organelles (FIG. 14a). The red-orange fluorescence indicates acridine orange accumulation and thus an acidified compartment. This fluorescent pattern is similar to that observed in various non-transformed cells such as the MCF-10F cells ( FIG. 14c), parietal cells [Berglindh et al., American Journal of Physiology, 238:G165-G176 (1980)], paramecium [Allen and Fok, J. Cell Biol., 97:566-570 (1983)], pituitary cells [Kreis et al., European Journal of Cell Biology, 49:128-139 (1989)] and Xenopus oocytes [Fagotto and Maxfield, J. Cell Sci., 107:3325-3337 (1994)]. The MCF-10F cells originated from a female patient with normal non-malignant breast tissue. These cells have a normal or near normal karyotype [Soule et al., Cancer Res., 50:6075-6086 (1990); Calaf and Russo, Carcinogenesis, 14:483-492 (1993)]. The cytosol and nucleoplasm of MCF-10F cells show a diffuse green fluorescence with discrete punctate red-orange organelles distributed throughout the cytoplasm. This pattern has been reported in many other non-transformed cells of non-mammary origin as well.

The acridine orange fluorescence in the drug-sensitive MCF-7 cells had, in contrast, significantly less red-orange fluorescent compartments indicating many fewer acidic vesicles (FIG. 14b). The fluorescence of acridine orange does not give any information as to the identity of the acidic compartments or the absolute value of the pH within these compartments. Therefore, subsequent experiments utilized ratiometric pH probes that could be targeted to specific organelles and whose pH could be calibrated in situ.

pH within the recycling endosome compartment. To measure the pH within the recycling endosome compartment, the cells were loaded with FITC transferrin. FITC is a fluorophore sensitive to pH within the range of 5.0 to 7.0 [Murphy et al., J. Cell Biol., 98:1757-1762 (1984)]. It was found that the drug-resistant MCF-7/ADR cells had an average recycling endosome compartment pH of 6.1±0.1, whereas the drug-sensitive MCF-7 cells had an average recycling endosome compartment pH of 6.6±0.1 (Table 1).

pH within the lysosomes: The pH of the lysosomes in MCF-7 and MCF-7/ADR cells was measured with LysoSensor Blue. The fluorescence of this probe is strongest at pH<5.1 and disappears at pH>5.8. As shown above, both MCF-7/ADR (FIG. 11b) and MCF-7 (FIG. 11a) cells were labeled with this probe and images then taken using the confocal microscope. In the MCF-7 cells there was virtually no fluorescence above background (FIG. 11a). This suggests that there are no compartments in MCF-7 cells with pH values below 5.8. On the other hand, when the MCF-7/ADR cells were labeled with the lysosomal pH probe, there were many fluorescent punctate organelles throughout the cytoplasm (FIG. 11b). The pH of the lysosomes in MCF-7/ADR cells was independently measured with FITC conjugated to dextran, a fluid phase probe that accumulates in lysosomes [Yamashiro and Maxfield, J. Cell Biol., 105:2723-2733 (1987)]. The cells were loaded for 30 minutes with FITC-dextran and the probe was subsequently chased for 90 minutes into the lysosomes. The average pH obtained for the lysosomes using this method was pH 5.1±0.1 (Table 1). This method could not be used for measuring the pH of lysosomes in MCF-7 cells because of the low uptake of the fluid phase marker. However the lack of acidic lysosomes as monitored by LysoSensor Blue in MCF-7 cells correlated with the lack of punctate Adriamycin fluorescence (FIG. 10b) as well as with the lack of punctate red fluorescence from acridine orange (FIG. 14b) within the cytoplasm of these cells. Therefore, while Lysosensor Blue failed to label lysosomes of MCF-7 cells (FIG. 11a), immunolocalization indicates that these cells have comparable numbers of lysosomes as MCF-7/ADR cells (FIG. 12). This demonstrates that the lysosomes are present in these drug-sensitive cells, but they fail to acidify.

pH within the cytoplasm and nucleoplasm. Earlier studies have shown that many drug-resistant cell lines have higher cytoplasmic pH than their drug-sensitive counterparts [Keizer and Joenje, J. Natl. Cancer Inst., 81:706-709 (1989); Boscoboinik et al., Br. J. Cancer, 61:568-572 (1990); Thiebaut et al., Journal of Histochemistry & Cytochemistry, 38:685-690 (1990); Roepe et al., Biochemistry, 33:11008-11015 (1994); Simon et al., Proc. Natl. Acad. Sci. USA, 91:1128-1132 (1994a); Simon and Schindler, Proc. Natl. Acad. Sci. USA, 91:3497-3504). In addition, changing the cytoplasmic pH by shiffing the pCO2 of the media results in a corresponding shift in cytoplasmic Adriamycin concentration [Example 1].When Adriamycin is incubated with cells, the first intracellular compartment into which it diffuses is the cytosol. The cytosolic pH determines the proportion of Adriamycin that will be protonated and unable to diffuse back across the plasma membrane.

All of these cytoplasmic pH measurements were made with cell permeant pH probes (e.g. BCECF-AM, SNARF-AM) that are de-esterified in the cytosol. There are a number of reasons, however, why these are not good probes for measuring cytosolic pH. First, some of the probes are internalized by endocytosis. Thus, they would be reporting a weighted sum of cytosolic and endosomal pH. Second, they accumulate in intracellular organelles which contain esterases and organic anion transporters that have been proposed to transport these probes [Di Virgilio et al., Cell Calcium, 11:57-62 (1990)]. Therefore, their fluorescence is a weighted average of the cytosolic and organellar pH. Third, these probes have been suggested as substrates for P-glycoprotein [Homolya et al., J. Biol. Chem., 268:21493-21496 (1993); Holló et al., Biochim. Biophys. Acta Bio-Membr., 1191:384-388 (1994)]. This would considerably complicate comparisons of results between the MCF-7 and MCF-7/ADR cells (which express P-glycoprotein).

To specifically measure the cytosolic and nucleoplasmic pH, an ideal probe would be large, membrane impermeable, and rapidly and selectively introduced into the cytosol. To generate such a probe, SNARF was conjugated to a dextran of 10 or 70 kd. The probe was scrape loaded into the cytosol by scraping adherent cells off the surface of polystyrene with a spatula [McNeil et al., J. Cell Biol., 98:1556-1564 (1984)]. The procedure takes place rapidly at 4° C., a temperature at which endocytic activity is minimal. The scraping causes a temporary shearing of the plasma membrane which allows the normally impermeant macromolecules to diffuse into the cytosol. Since the pH probe SNARF is conjugated to a dextran, once introduced into the cytosol it does not cross cellular membranes. To measure the pH within the cytosol, the probe was conjugated to a 70 kD dextran which is too large to pass through the nuclear pores (FIG. 15a). To measure the pH within the nucleoplasm, the probe was conjugated to a 10 kD dextran which is too large to cross membranes into organelles, but still small enough to pass through the nuclear pores (FIG. 15b).

The results of these measurements show that MCF-7 cells have an average cytosolic pH 6.75+/−0.3 which is 0.4 units lower than the cytosolic pH of MCF-7/ADR cells (pH 7.15+/−0.1) when the extracellular medium is buffered at pH 7.3 (Table 1). The pH of the nucleoplasm in both cell types was 0.1-0.3 pH units more alkaline than the cytosol pH (Table 1). This is consistent with other recent reports that find a more alkaline nucleoplasmic pH [Seksek and Bolard, J. Cell Sci., 108:1291-1300 (1996)]. The higher cytosolic pH of MCF-7/ADR cells results in a lower plasma membrane ΔpH and contributes to a greater organellar ΔpH. This is consistent with the hypothesis that the distribution of the weak base Adriamycin (FIG. 10) is affected by pH gradients across membranes.

Effect of ionophores and blockers of the H+-ATPase on the response of MCF-7/ADR cells to Adriamycin: The results presented above demonstrate a large difference in the subcellular pH profile between the drug-resistant and drug-sensitive cell types. The PSS hypothesis predicts that these differences in acidification are causally related to the drug-resistance phenotype. To test this hypothesis, experiments were performed to test whether changing the subcellular pH profile of drug-resistant MCF-7/ADR cells to that of drug-sensitive MCF-7 cells would affect the distribution of the chemotherapeutic drug Adriamycin. To do this, the sodium/proton exchanger monensin [Tartakoff and Vassalli, J. Cell Biol., 79:694-707 (1978); Maxfield, J. Cell Biol., 95:676-681 (1982); Mollenhauer et al., Biochim. Biophys. Acta, 1031:225-246 (1990); Schindler et al., Biochemistry, 35:2811-2817 (1996)] and the proton ATPase inhibitors Bafilomycin A1 and Concanomycin A [Maxfield, J. Cell Biol., 95:676-681 (1982); Dröse and Altendorf, Journal of Experimental Biology, 200:1-8 (1997)] were employed to block acidification. Acridine orange labeled MCF-7/ADR cells (FIG. 16a) when treated with monensin (FIG. 16b) did not exhibit the red spectral shift within the vesicles, indicating the loss of acidification in these compartments. Similar results were seen with the potassium/proton-exchanger nigericin. Likewise, after MCF-7/ADR cells labeled with acridine orange (FIG. 17a, 17e) were incubated with Bafilomycin A1 (FIG. 17b) or Concanomycin A (FIG. 17f), the punctate red labeling of acidic organelles was lost. None of these agents have any direct effect on the fluorescent properties of acridine orange.

The distribution of Adriamycin in MCF-7/ADR cells (FIG. 16c, 17c, 17g) was monitored upon addition of either monensin (FIG. 16d) or Bafilomycin A1 (FIG. 17d) or Concanomycin A (FIG. 17h). Treatment with monensin, Bafilomycin A1 or Concanomycin A, redistributed Adriamycin to the nucleoplasm. It should be noted that there is also a simultaneous decrease in the perinuclear accumulation of Adriamycin. This distribution of Adriamycin is similar to that observed in the drug-sensitive MCF-7 cells (FIG. 10b).

Discussion

Most chemotherapeutic agents have sites of action in the nucleus or in the cytosol. Therefore, their toxicity depends upon their concentration in either of these two compartments. The protonation, sequestration and secretion (PSS) hypothesis proposes that the concentration of weak base chemotherapeutics in both the cytosol and nucleoplasm is regulated by the ability of cytoplasmic organelles to sequester the drugs away from the cytosol. The PSS hypothesis is based on the assumption that chemotherapeutic drugs entering acidic organelles should become protonated, thereby sequestered from the cytosol, and secreted. Adriamycin co-localizes on the light microscopic level with the acidic organelles of living drug-resistant MCF-7/ADR cells including the lysosomes, recycling endosome compartment, and the TGN. This result is consistent with an ultrastructural study of drug resistant NIH 3T3 cells which showed that the golgi and lysosomes were labeled by a photo-precipitate of Adriamycin. [Rutherford and Willingham, Journal of Histochemistry and Cytochemistry, 41:1573-1577 (1993)].

Chemotherapy relies upon tumor cells being more sensitive to chemotherapeutics than non-transformed cells. One factor that contributes to this enhanced sensitivity is a failure of the PSS mechanism in tumor cells. The results presented here demonstrate two aberrations of cellular pH regulation in the MCF-7 drug-sensitive tumor cells: First, there is a failure to acidify organelles as measured both qualitatively (FIG. 14) and quantitatively (Table 1). Second, the cytosol in MCF-7 cells is 0.4 pH units more acidic than the cytosol of MCF-7/ADR cells. As described below, both of these features will increase the concentration of chernotherapeutics in the cytosol and nucleoplasm of drug-sensitive tumor cells relative to the concentrations in drug-resistant or non-transformed cells.

Effects of plasma membrane ΔpH on drug-distribution: The pH gradient across the plasma membrane of MCF-7 cells is 0.55 units whereas in drug-resistant MCF-7/ADR cells it is 0.15 units. Therefore at equilibrium conditions, there will be approximately 2.5-fold greater accumulation of Adriamycin in the cytosol of MCF-7 cells than in that of MCF-7/ADR cells.

Effects of organelle membrane ΔpH on drug-distribution. The pH gradients across the organelles of the MCF-7 cells was almost zero (Table 1). Thus the organelles could not accumulate chemotherapeutics. In contrast, the pH gradient between the recycling endosome compartment and the cytosol of MCF-7/ADR is calculated to be an average of 1.0 pH unit, which would cause a ten-fold accumulation in this organelle. The ΔpH between the lysosome and cytosol in MCF-7/ADR cells is greater than 2.0 pH units. Thus, the lysosomes of MCF-7/ADR cells should accumulate at least a 100-fold more Adriamycin than the cytosol.

If the distribution of Adriamycin reached an equilibrium, then the cytosolic concentration would be solely dependent on the ΔpH across the plasma membrane and extracellular drug concentration. Acidic organielles would accumulate high levels of drugs, but could not lower the cytosolic concentration. However, a large body of work has shown that many acidic organelles including the TGN and recycling endosomes continuously secrete their contents by exocytosis. This active process, if fast relative to diffusion of extracellular drug into the cytosol, will keep cytosolic and nuclear drug levels low. Drug concentrations would not reach equilibrium distribution but remain at a steady state due to the continuous cycling of acidic organelles. Thus organellar acidification would lower the concentration of Adriamycin in the cytosol and nucleoplasm Of drug-resistant and non-transformed cells. In fact this mechanism can account for the difference in Adriamycin distribution observed between MCF-7 and MCF-7/ADR cells (FIG. 10). In drug-resistant MCF-7/ADR cells, Adriamycin was sequestered within subcellular organelles, decreasing the drug concentration within the nucleoplasm and, accordingly in the cytosol as well (The high density of organelles throughout the cytoplasm makes it impossible to resolve Adriamycin fluorescence selectively from the cytosol. The nucleoplasmic concentration approximately reflects the free cytosolic concentration since Adriamycin should be freely permeable through both the nuclear envelope and the nuclear pores, whose size cut-off is 25 nm [Feldherr et al., J. Cell Biol., 99:2216-2222 (1984)]). In drug-sensitive cells, in the absence of an organellar mechanism for sequestration, a greater percentage of incoming Adriamycin remained in the cytosol with access to binding sites within the nucleus.

The PSS hypothesis proposes that cytosolic/nucleoplasmic drug concentrations are a function of the ΔpH and drug-permeability of the plasma membrane and the ΔpH, and drug-permeability of the organellar membrane and the rate of exocytosis. Hence nuclear/cytosolic drug levels would be increased by: 1) elevated plasma membrane ΔpH which would increase cytosolic drug accumulation; 2) decreased organellar ΔpH which would decrease sequestration, and 3) decreased rate of secretion which would permit the drug levels to equilibrate across organelle membranes. The PSS hypothesis predicts that, at a steady state, dissipation of plasma membrane ΔpH will decrease drug-sensitivity whereas dissipation of organellar ΔpH and/or decreasing the rate of secretion will increase drug-sensitivity.

Multidrug resistance in tumors could stem from a number of cell biological changes. The most frequently proposed mechanism for MDR in tumors is a plasma membrane based efflux pump that utilizes ATP to transport chemotherapeutics [Gottesman and Pastan, Annu. Rev. Biochem., 62:385-427 (1993)]. This idea is based on studies in cell lines that express two of the proteins implicated in multidrug resistance: Pglycoprotein (Pgp) and the Multidrug resistance associated protein (MRP). The evidence includes the observations that: (1) addition of azide to these cells increases nuclear accumulation of chemotherapeutics, (2) both Pgp and MRP have ATP-binding domains; (3) chemotherapeutic drugs modified with photoactive groups can be used to label Pgp. The PSS mechanism tested in this paper may be an additional mechanism for drug-resistance working separately from the Pgp and MRP drug-efflux pumps.

It is also possible that both Pgp and MRP contribute to the PSS mechanism by affecting organelle pH. Indeed, it has been demonstrated that tranfection of cells with Pgp results in an alkaline shift of the cytosolic pH [Thiebaut et al., Journal of Histochemistry & Cytochemistry, 38:685-690 (1990)] consistent with the pH shift seen in resistant cells (Table 1). One clue to the mechanism for this pH shift may come from the structural homology between Pgp, MRP and the ATP binding cassette family of proteins which includes the cystic fibrosis transmembrane conductance regulator (CFTR), a chloride channel expressed in lung epithelial cells. In cells defective in CFTR, there is reduced acidification of the Golgi and recycling endosome compartments [Barasch et al., Nature (London), 352:70-73 (1991)]. Acidification of organelles requires a proton ATPase. However, proton pumping alone is not sufficient to acidify to levels seen in organelles such as endosomes and lysosomes. The membrane potential caused by the proton gradient blocks the activity of the proton ATPase. Therefore anion channels are necessary to bring neutralizing negative charge to dissipate the membrane potential and keep the proton ATPase pumping protons [Glickman et al., J. Cell Biol., 97:1303-1308 (1983); Al-Awqati et al., Society of General Physiologists Series, 44:283-294 (1989)]. Both Pgp and MRP have been observed to be components of intracellular membranes [Baldini et al., European Journal of Cell Biology, 68:226-239 (1995); Molinari et al., Int. J. Cancer, 59:789-795 (1994)]. They might either form ion channels or modify the activity of ion channels [Abraham et al., Proc. Natl. Acad. Sci. USA, 90:312-316; Diaz et al., Pflugers Arch., 422:347-353 (1993); Ehring et al., J. Gen. Physiol., 104:1129-1161 (1994); Gill et al., Cell, 71:23-32 (1992); Hainsworth et al., Pflugers Archiv.—European Journal of Physiology, 432:234-240 (1996); Hardy et al., EMBO Journal, 14:68-75 (1995); Jirsch et al., Cancer Res., 53:4156-4160 (1993); Thévenod et al., J. Biol. Chem., 269:24410-24417 (1994); Valverde et al., Nature (London), 355:830-833 (1992)]. If the MCF-7 cells were missing a counter-ion transport, this would limit acidification of the organelles. The presence of Pgp or MRP may facilitate a counter ion transport allowing restoration of acidification within these organelles in the MCF-7/ADR cells. However, there is still disagreement over whether Pgp forms an ion-channel [Dong et al., Cancer Res., 54:5029-5032 (1994); Ehring et al., J. Gen. Physiol., 104:1129-1161 (1994); Tominaga et al., J. Biol. Chem., 270:27887-27893 (1995)].

There are differences in the pH gradients across both the plasma membrane and organellar membranes between drug-sensitive and -resistant cells (Table 1). Results presented here indicate that the cellular organelles are critical determinants of cellular sensitivity to weak base chemotherapeutics. Monensin disrupts pH gradients across all cellular membranes [Mollenhauer et al., Biochim. Biophys. Acta, 1031:225-246 (1990)]. Dissipation of plasma membrane ΔpH should decrease the cytosolic concentration of Adriamycin in MCF-7/ADR cells and, conversely, dissipation of organellar ΔpH should increase cytosolic and nucleoplasmic Adriamycin. The observation that monensin increases the nucleoplasmic concentration of Adriamycin suggests that organellar pH gradients make a greater contribution to Adriamycin distribution than plasma membrane pH gradients. Further, both Bafilomycin A1 and Concanomycin A, which specifically block the vacuolar proton ATPase in endosomes and lysosomes of eukaryotic cells [Dröse and Altendorf, Journal of Experimental Biology, 200:1-8 (1997); Maxfield, J. Cell Biol., 95:676-681 (1982)] and Golgi [Kim et al., J. Cell Biol., 134:1387-1399 (1996)], increase Adriamycin levels in the nucleus (FIG. 8d,h). Thus, the drug-resistant phenotype—both sequestration of drugs into cytoplasmic organelles and the sensitivity of cells to the weak-base chemotherapeutics—is causally dependent upon organelle acidification. The pH gradient across the plasma membrane may make significant contributions to the sensitivity of drug resistant cells to non-weak base chemotherapeutic drugs such as colchicine and taxol. The binding of colchicine to tubulin is pH dependent and is favored at more acidic pH [Mukhopadhyay et al., Biochemistry, 29:6845-6850 (1990)]. Similarly, an acidic pH favors the stabilization of microtubules by taxol [Ringel and Horwitz, Journal of Pharmacology & Experimental Therapeutics, 259:855-860 (1991)]. Thus, the acidic cytoplasmic pH of tumor cells increases the activity of chemotherapeutic drugs. The more neutral pH of non-transformed and MDR cells decreases their activity.

In the PSS model, acidification of organelles plays a direct role in the accumulation of weak base chemotherapy agents. Currently, it is not known if sequestering chemotherapeutic drugs within subcellular organelles followed by secretion is sufficient to account for all the drug resistance of MCF-7/ADR cells. However, disrupting the acidification of drug-resistant MCF-7/ADR cells with the protonophore monensin or H+-ATPase blockers Bafilomycin A1 and Concanomycin A reverses the subcellular Adriamycin distribution to that seen in drug-sensitive MCF-7 cells (FIGS. 16, 17) and increases Adriamycin toxicity [Schindler et al., Biochemistry, 35:2811-2817 (1996)]. Thus, if there are mechanism(s) of resistance that do not depend upon organellar pH, then they are not sufficient to maintain drug-resistance.

TABLE 1
Summary of endosome, lysosome, cytosol and nucleoplasm pH

| compartment | endosome | lysosome | lysosome | cytosol | nucleoplasm |
|---|---|---|---|---|---|
| probe | FITC- | FITC- | Lysosensor | SNARF-70 kD | SNARF-10 kD |
| | transferrin | dextran | Blue | dextran | dextran |
| pH MCF-7 | 6.6 ± 0.1 | ND | >>5.8 | 6.75 ± 0.3 | 7.1 +/− 0.1 |
| ΔpH to cytosol | 0.15 | ND | <<0.9 | — | −0.35 |
| ΔpH to media | 0.7 | ND | <<1.5 | 0.55 | 0.2 |
| pH MCF-7/ADR | 6.1 ± 0.1 | 5.1 ± 0.1 | <<5.8 | 7.15 ± 0.1 | 7.2 +/− 0.1 |
| ΔpH to cytosol | 1.05 | 2.05 | >>1.35 | — | −0.05 |
| ΔpH to media | 1.2 | 2.2 | >>1.5 | 0.15 | 0.1 |

### EXAMPLE5

### Agents that Reverse Adriamycin Resistance in Human Breast Cancer Cells Disrupt Cellular Mechanisms of Drug Sequestration and Exocytosis

| | Abbreviations |
|---|---|
| CHO: | Chinese Hamster Ovary |
| DAMP: | N-(3-((2,4-dinitrophenyl)amino)propyl)-N- |
| | (3-aminopropyl)methylamine dihydrochioride |
| DME: | Dulbecco's modified eagle medium |
| DNP: | Dinitrophenol |
| FBS: | Fetal bovine serum |
| FITC: | Fluorescein-5-isothiocyanate |
| HBSS: | Hanks balanced salt solution |
| MDR: | Multidrug resistance |
| MRP: | Multidrug resistance associated protein |
| NBD: | Nitrobenz-2-oxa-1,3-diazole |
| PBS: | Phosphate buffered saline |
| Pgp: | P-glycoprotein |
| PSS: | Protonation, sequestration and secretion |
| SNARF: | Seminaphthorhodafluor |
| TGN: | trans-Golgi network |

Introduction

Treating tumors with chemotherapeutics can result in tumor resistance to a spectrum of cytotoxic drugs. This multidrug resistance (MDR) is a major challenge to the successful treatment of many cancers [Simon and Schindler, Proc. Natl. Acad. Sci. USA, 91:3497-3504 (1994b)]. Experimental evidence supports a variety of mechanisms for MDR including: a) reduced drug influx from altered composition of membrane phospholipids; b) increased drug efflux from increased activity of plasma membrane transporters; c) an increased sequestration of drugs in cytoplasmic organelles away from the cytosol and nucleoplasm; d) increased drug efflux by exocytosis of drugs sequestered in the secretory pathway; e) reduced drug accumulation as a consequence of a shift of cytosolic pH or plasma membrane potential; f) increased drug detoxification; g) mutations in drug targets; h) defects in the apoptosis response to cellular damage [Simon and Schindler, Proc. Natl. Acad. Sci. USA, 91:3497-3504 (1994b). Any or all of these mechanisms may contribute to the MDR phenotype.

The primary chemotherapeutic drug for treating breast cancer is Adriamycin (doxorubicin), a heterocyclic amine with a pKa of 8.3. The intrinsic fluorescence of Adriamycin allows its distribution in living cells to be visualized and quantified [Simon et al., Proc. Natl. Acad. Sci. USA, 91:1128-1132 (1994a); Schindler et al., Biochemistry, 35:2811-2817 (1996)]. In drug-sensitive tumor cells, Adriamycin distributes throughout the cytoplasm and accumulates within the nucleus, binding to nucleic acids and topoisomerase II [Simon et al., Proc. Natl. Acad. Sci. USA, 91:1128-1132 (1994a); Simon & Schindler, Proc. Natl. Acad. Sci. USA, 91:3497-3504 (1994b); Schindler et al., Biochemistry, 35:2811-2817 (1996)]. In contrast, Adriamycin is not observed in the nucleus and accumulates only within intracellular vesicular compartments of drug-resistant human breast cancer (MCF-7/ADR) cells [Schindler et al., Biochemistry, 35:2811-2817 (1996)] and of many other drug-resistant cell lines [Willingham et al., Cancer Res., 46:5941-5946 (1986); Hindenburg et al., Cancer Res., 49:4607-4614 (1989); Weaver et al., Exp. Cell Res., 196:323-329 (1991); Lankelma et al., Biochim. Biophys. Acta Mol. Cell Res., 1093:147-152 (1991); Gervasoni, Jr., et al., Cancer Res., 51:4955-4963 (1991); Jaffrézou et al., Cancer Res., 52:6440-6446 (1992); Coley et al., Br. J. Cancer, 67:1316-1323 (1993); Rutherford and Willingham, Journal of Histochemistry and Cytochemistry, 41:1573-1577 (1993)]. Adriamycin is relatively membrane-permeant in its neutral form and relatively impermeant when protonated [Dalmark and Storm, The Journal of General Physiology, 78:349-364 (1981); Mayer et al., Biochim. Biophys. Acta, 857:123-126 (1986)]. Thus, it accumulates within acidic organelles such as the lysosomes [Rutherford and Willingham, Journal of Histochemistry and Cytochemistry, 41:1573-1577 (1993); Schindler et al., Biochemistry, 35:2811-2817 (1996)], the acidic perinuclear recycling compartment (PRC) and the acidic trans-Golgi network (TGN) [Schindler et al., Biochemistry, 35:2811-2817 (1996); Example 4, above]. These compartments appear to sequester the drug from the nucleus, since little Adriamycin fluorescence is observed within the nucleoplasm.

In the MCF-7 Adriamycin-sensitive human breast cancer line the intracellular vesicular compartments do not acidify [Schindler et al., Biochemistry, 35:2811-2817 (1996); Example 4, above]. In marked contrast, normal organelle acidification is observed in MCF-7/ADR drug-resistant cells [Schindler et al., Biochemistry, 35:2811-2817 (1996); Example 4, above]. This facilitates the protonation and sequestration of Adriamycin within these organelles. Chemotherapeutic drugs sequestered in acidic secretory organelles are rapidly secreted from the cell through normal exocytosis of the recycling endosome and biosynthetic pathways [Beck, Biochem. Pharm., 36:2879-2887 (1987); Sehested et al., Biochem. Pharmacol, 37:3305-3310 (1988); Simon and Schindler, Proc. Natl. Acad. Sci. USA, 91:3497-3504 (1994b); Schindler et al., Biochemistry, 35:2811-2817 (1996)]. This mechanism of protonation, sequestration and secretion (PSS model) of chemotherapeutics within the efflux pathway should not be functional in the acidification-deficient drug-sensitive cells [Schindler et al., Biochemistry, 35:2811-2817 (1996)].

The PSS mechanism for drug resistance makes the following testable predictions: 1) chemotherapeutics should accumulate in the acidic organelles of drug-resistant cells and diffuse through the cytosol of drug-sensitive cells; 2) the intracellular organelles of drug-sensitive cells should either be reduced in acidification or slowed in their transport to the cell surface; 3) agents that disrupt organelle acidification (protonophores such as monensin, nigericin, or blockers of the H+-ATPase) should reverse the drug resistance of MDR tumor cells [Simon et al., Proc. Natl. Acad. Sci. USA, 91:1128-1132 (1994a); Simon and Schindler, Proc. Natl. Acad. Sci. USA, 91:3497-3504 (1994b); Schindler et al., Biochemistry, 35:2811-2817 (1996)], 4) agents that disrupt transport of organelles to the plasma membrane (e.g., inhibitors of ceramide synthase) should reverse the drug resistance of MDR tumor cells, and 5) agents that reverse the drug-resistance of MDR tumor cells should disrupt either organelle acidification or transport through the exocytotic pathway. The first three predictions have recently been tested [Schindler et al., Biochemistry, 35:2811-2817 (1996); Example 4, above]. The secretory organelles of drug-sensitive MCF-7 cells are not acidified and do not accumulate chemotherapeutics. Agents that disrupt acidification of the organelles of the drug-resistant MCF-7/ADR cells disperse the chemotherapeutic drugs from the organelles, increase the cytoplasmic and nucleoplasmic concentrations of chemotherapeutic drugs, and sensitize drug-resistant cells to chemotherapeutics. This demonstrates a causal relationship between drug-sensitivity and acidification of the exocytotic pathway. If there are other mechanisms for Adriamycin-resistance in these cells, they are not sufficient to maintain drug resistance.

The experiments presented here were designed to test the fifth prediction of the PSS hypothesis: Agents that reverse drug resistance should block acidification of cytoplasmic organelles or their transport to the surface. Many drugs have been reported to reverse drug-resistance in vitro. These include compounds that interact with the estrogen receptor (tamoxifen and diethylstilbestrol, DES), blockers of calcium signaling (trifluoroperizine, verapamil and nifedipine), phosphatase inhibitors (cyclosporin A and FK-506), and assorted others (progesterone and reserpine). This report demonstrates that three representative molecules, verapamil, cyclosporin A and tamoxifen, block organelle acidification at the same concentrations at which they block drug-resistance. Most of the results focus on the effects of tamoxifen, demonstrating that it directly blocks acidification of organelles and slows transport of the exocytotic pathways within drug-resistant MCF-7/ADR cells.

It is proposed that acidification of secretory pathways may be a universal mechanism for protecting cells from alkaloids—environmental toxins. The sensitivity of tumor cells to chemotherapeutics is a consequence of a pathology of the exocytotic pathways: disruption of the lumenal pH within the PRC and TGN. Multidrug resistance can be considered a reversal of these defects. Blocking acidification of this pathway by tamoxifen blocks secretion of the chemotherapeutic drugs and reverses drug-resistance. However, a potentially serious complication of tamoxifen treatment is that it may also block the ability of cells to rid themselves of environmental toxins and carcinogens, such as diethylnitrosamines, which are weak bases. These results have implications for the recent use of tamoxifen as a prophylactic treatment for cancer.

Materials and Methods

Materials: Bafilomycin A1, monensin, acridine orange, tamoxifen, verapamil, cyclosporin A, Tris-ATP and nigericin were from Sigma (St. Louis, Mo.). BODIPY®-transferrin, BODIPY®-ceramide, Lysosensor Blue DND-167, fluorescein-5-isothiocyanate (FITC)-transferrin, seminaphthorhodafluor (SNARF)-dextran, N-(3-((2,4-dinitrophenyl)amino)propyl)-N-(3-aminopropyl)methylamine, dihydrochloride (DAMP), NBD-ceramide, and FITC-dextran were from Molecular Probes (Eugene, Oreg.). Adriamycin was from Calbiochem (La Jolla, Calif.). Concanomycin A was from Fluka (Milwaukee, Wis.). Mouse anti-dinitrophenol (DNP) antibody was from Oxford (Oxford, Mich.). Gold conjugated anti-mouse secondary antibody was from Amersham (Arlington Heights, Ill.).

Cell Culture: Cells were seeded and grown in Dulbecco Modified Eagle's (DME) medium containing 10% fetal calf serum (phenol red free) in Lab-Tek coverslip culture chambers (Nunc, Naperville, Ill.) or on coverslips and maintained in an incubator at 37° C. and 5% CO2. Human breast cancer cells (MCF-7, MDA-231) and the Adriamycin-resistant lines (MCF-7/ADR, MDA-A1) were obtained from Dr. William W. Wells of the Dept. of Biochemistry, Michigan State University and the American Type Culture Collection. The medium for the MCF-7/ADR cells was supplemented with Adriamycin (0.5 μg/ml). Cells were utilized 3-4 days following plating.

Cell Viability Assays: Cell viability was assayed by plating cells at ˜1000 cells/well in 24 well plates (Falcon) in DME. After 60 hours, the cells were incubated in fresh medium that was supplemented with various concentrations of Adriamycin, and tamoxifen (solubilized in ethanol at 50 mM) for 6 hours, then washed and placed in fresh drug-free medium. The cells were fed daily for three days and then cell viability was quantified with two independent techniques: an assay of total DNA and an assay of volume of viable cells. The DNA content of the adherent cells was quantified fluorometrically by Hoechst 33258 which undergoes a ten fold increase in fluorescence upon binding DNA: The cells were washed twice on the 24-well plates with Hanks Balanced Salt Solution (HBSS, phenol red free) to remove unattached cells, placed in hypotonic medium (0.1×HBSS) and sonicated for 30 seconds. The homogenate from each well was collected and Hoechst 33258 was added to a final concentration of 1 μg/ml. Fluorescence was measured on an SLM Aminco-Bowman series 2 luminescence spectrometer with a λex=356 nm and a λem=492 nm. Calf thymus DNA was used for calibration.

Microscopy: Unless otherwise indicated, all cells (optical sections at 0.5 μM intervals) were examined at 37° C. with an Ultima Laser Scanning Confocal Microscope (Meridian Instruments, Okemos, Mich.) [Simon et al., Proc. Natl. Acad. Sci. USA, 91:1128-1132 (1994a)]. In some experiments cells were observed under epi-fluorescence using a Olympus IX-70 inverted microscope with xenon arc lamp excitation and a cooled CCD camera (Hamamatsu Photonics Model 4742-95, Hamamatsu City, Japan) or a Nikon Diaphot inverted microscope with mercury arc lamp excitation and an intensified CCD camera (Hamamatsu Photonics Model C5909, Hamamatsu City, Japan). Images were collected and analyzed with software written in LabView (National Instruments, TX, USA).

Cells growing on Lab-Tek chambers were mounted on the microscope under a 37° C. heater and superfused with moist 5% CO2. Cells growing on coverslips were mounted on the microscope with constant perfusion of medium at 37° C. and equilibrated with 5% CO2. The cells remained stable for many hours allowing the effects of a variety of media and reagents to be assayed on the same field of cells.

Labeling of Cells with Adriamycin: Cells were loaded with Adriamycin (10 μM) added to the perfusion medium. On the confocal microscope Adriamycin fluorescence was imaged with excitation at 488 nm using an atrgon ion laser (Coherent, Santa Clara, Calif.) and a 60×1.4 NA oil immersion objective. For epi-fluorescence, the cells were excited with 450-490 nm filter and emission was monitored with a 510 nm longpass filter.

Labeling of Cells with Acridine Orange: To examine intracellular acidic compartments, acridine orange (6 μM in medium from a 10 mM stock in water) was added directly to cells in the Lab-Tek chambers and the cells were incubated for 15 minutes. Cells in the presence of acridine orange were then examined utilizing an excitation at 488 nm and dual emission confocal images were simultaneously recorded using both a 530-30 band pass barrier filter (green fluorescence) and a 605 nm long pass barrier filter (red fluorescence). Optical sections of the fluorescent samples were recorded at 0.5 micron intervals with a 60×oil immersion objective.

Organelle-specific pH measurements: The pH was measured in selective cellular compartments by targeting ratiometric pH probes to specific organelles. Using the confocal microscope, the pH probe SNARF was excited at 514 nm and its emission was recorded simultaneously on two orthogonal PMT's using a 610 nm dichroic a 570/30 nm bandpass filter and a 630 nm longpass filter. Using a Nikon epifluorescence microscope with a intensified CCD camera, the pH probe FITC was excited alternately at 450 nm and 490 nm and emission recorded with a 520/10 bandpass filter. At the end of each experiment the emission profiles of the dyes were calibrated as a function of pH as previously described in Example 4, above. In short, to calibrate the pH for the endosomal system, the chamber was perfused with 150 mM sodium buffers at pH of 5, 6 or 7 containing of monensin (20 μM) and nigericin (10 μM) for 5 minutes before recording the fluorescence. For the pH calibrations of cytosol and nucleoplasm, the cells were incubated in 140 mM potassium buffers at pH 6.0, 6.5, 7.0 and 7.5 containing nigericin (20 μM).

pH in the recycling endosomes: The transferrin receptor has been used as a selective probe for the recycling endosome pathway [Fuller and Simons, J. Cell Biol., 103:1767-1779 (1986); Roff et al., J. Cell Biol., 103:2283-2297 (1986); Sipe and Murphy, Proc. Natl. Acad. Sci. USA, 84:7119-7123 (1987); Stoorvogel et al., J. Cell Biol., 106:1821-1829 (1988); Dunn et al., J. Cell Biol., 109:3303-3314(1989); Mayor et al., J. Cell Biol., 121:1257-1269(1993); McGraw et al., J. Cell Biol., 155:579-594 (1993)]. After endocytosis, the transferrin is transported through the endosomes and then recycled back to the surface without passage through the lysosomes. Thus, the pH of the recycling endosomes can be selectively monitored by conjugating a pH probe, such as FITC or SNARF, to transferrin [Dunn et al., J. Cell Biol., 109:3303-3314 (1989)] Example 4, above]. Cells growing in Lab-Tek chambers were incubated with FITC-transferrin (150 μg/ml in DME/20 mM HEPES, pH 7.3) for 25 minutes, washed in quick succession 3× with DME/HEPES and 3× with HBSS/HEPES, and imaged [Ghosh and Maxfield, J. Cell Biol., 128:549-561 (1995)].

pH in the lysosomes: The pH in the lysosomes was assayed both with light and electron microscopy. Light microscopy: Cells were incubated with FITC-dextran 10 kD (5 mg/ml) (DME/HEPES) for 30 minutes, washed 4× with DME/HEPES, incubated for an additional 90 minutes to chase out the endosomes and visualized on a Nikon Diaphot equipped with FITC excitation filters (see above) [Yamashiro and Maxfield, J. Cell Biol., 105:2723-2733 (1987)]. The pH was calibrated as described above. Electron microscopy: The cells were incubated with the weak base DAMP, fixed, probed with an mouse antibody to DNP (cross-reacts with DAMP) and visualized with gold-conjugated anti-mouse antibodies. This has been used to quantify the pH in different cellular organelles and the technique was used as previously published [Barasch et al., J. Cell Biol., 107:2137-2147 (1988); Barasch et al., Nature (London), 352:70-73 (1991)]. In short, cells growing on 35 mm dishes were incubated with 0 μM or 10 μM tamoxifen (DME/HEPES/37° C./5% CO2) for 45 minutes. Then DAMP was added to a final concentration of at 70 μM and the cells were incubated for another 45 minutes. The medium was replaced with phosphate buffered saline (PBS), pH 7.4, with 4% paraformaldehyde and 0.75% gluteraldehyde. The cells were incubated one hour at room temperature, washed with several changes of PBS pH 7.4 containing 50 mM NH4Cl. After at least eight hours, the cells were scraped off the dish, pelleted, and placed in 70% ethanol for 15 minutes, 17.5% ethanol:75% LR White overnight, and 100% LR White for 24 hours. The cells were embedded in LR White in gelatin capsules and baked at 60° C. for 24 hours in a vacuum oven, sectioned and incubated overnight at 4° C. with anti-DNP antibodies in 4% FBS, washed and incubated in gold (10 nm) labeled secondary antibodies for 2 hours. They were then stained and visualized under the electron microscope.

pH in the cytosol: The pH in the cytosol was selectively assayed by using the ratiometric pH probe SNARF conjugated to 70 kD dextran which was scrape loaded into the cytoplasmic compartment [McNeil et al., J. Cell Biol., 98:1556-1564 (1984); Example 4, above]. The 70 kD dextran is too large to enter into organelles or the nucleus. Cells were plated on polystyrene plates at 50% confluency. Twenty-four to thirty-six hours later, the medium was aspirated and the cells were covered with 50 μl of DME with SNARF-dextran (10 mg/ml). The cells were quickly scraped off the polystyrene and placed in pre-chilled tubes containing 1 ml DME without serum. The cells were harvested (100 g for 5 minutes), washed twice with pre-chilled DME, and plated on Lab-Tek chambers in DME with serum. The cells were allowed to recover for 24 hours prior to examination on a confocal microscope. The pH was calibrated from SNARF fluorescence as described above.

pH in the nucleus: The nucleoplasmic pH was probed by loading the cytosol with SNARF conjugated to a 10 kD dextran. This dextran is too large to cross cellular membranes, but can enter the nucleoplasm by diffusion across the nuclear pores. Confocal fluorescence microscopy was used to prepare optical sections through the cell allowing the fluorescence intensity of the nucleoplasm and cytoplasm to be quantified. The 10 kD SNARF-dextran was loaded into the cytosol and imaged using the same techniques for the 70 kD SNARF-dextrans (see above).

Transport Assays: Transport of transferrin from recycling endosomes to cell surface Transferrin has been used to selectively label the recycling endosomes of cells [Fuller and Simons, J. Cell Biol., 103:1767-1779(1986); Roff et al., J. Cell Biol., 103:2283-2297 (1986); Sipe and Murphy, Proc. Natl. Acad. Sci. USA, 91:3497-3504 (1987); Stoorvogel et al., J. Cell Biol., 106:1821-1829(1988); Dunn et al., J. Cell Biol., 109:3303-3314(1989); Mayor et al., J. Cell Biol., 121:1257-1269 (1993); McGraw et al., J. Cell Biol., 155:579-594 (1993)]. The endocytic pathway is known to undergo acidification. Thus, the fluorophore BODIPY was used as a probe on transferrin since its fluorescence is photostable and insensitive to pH. Transport of transferrin was assayed as previously described [Ghosh and Maxfield, J. Cell Biol., 128:549-561 (1995)]. Cells growing in Lab-Ten chambers were loaded with 25 μg/ml BODIPY-transferrin (DME/HERES/pH 7.3/37° C.). After 20 minutes, the medium was replaced with citric acid buffer (25.5 mM citric acid monohydrate, 24.5 mM sodium citrate, 280 mM sucrose, pH 4.6) containing 10 μM deferoxamine mesylate and incubated for two minutes at 37° C. to remove plasma membrane bound BODIPY-transferrin. The cells were rapidly washed 4 times with McCoys 5A medium (20 mM HERES, 50 μM deferoxamine mesylate and 100 μg/ml unlabeled human transferrin) and examined on a confocal microscope λex=488, λem=530) at various time points.

Transport of sphingomyelin from TGN to cell surface: BODIPY-ceramide labels endomembranes and its metabolic product, BODIPY-sphingomyelin, accumulates within the Golgi compartments [Pagano et al., J. Cell Biol., 113:1267-1279 (1991)]. When accumulated at high concentrations, BODIPY-sphingomyelin undergoes a green to red shift in fluorescence emission. Excitation was at 488 nm and dual emission images were prepared utilizing the filter set described for acridine orange and a 100×oil immersion objective.

Efflux studies with BODIPY-ceramide were performed in the following manner: Cells cultured for 3-4 days in Lab-Ten chambers were washed three times with DME (pH 7.2), incubated with BODIPY-ceramide (3 μg/ml) for 60 minutes at 37° C./5% CO2, washed two times with cold DME, and then incubated in the absence or presence of Tamoxifen (10 μM) for 15 minutes on ice. The cells were then incubated for 0, 60, or 120 minutes at 37° C./5% CO2 in DME or DME/Tamoxifen (10 μM), fixed, and imaged. For 60 and 120 minute time points, medium was replaced with pre-warmed DME or DME/Tamoxifen every 30 minutes. Cells were fixed by washing two times in cold fixing buffer (1% paraformaldehyde, 0.1 M sodium cacodylate, 0.1 M sucrose, pH 7.2) followed by a 15 minute incubation in fixing buffer on ice, and then washing two times with cold fixing buffer without paraformaldehyde. Attempts to use 2-5% albumin to back exchange externalized labeled BODIPY-ceramide or BODIPY-sphingomyelin proved difficult because of the occurrence of high fluorescent backgrounds. These probably resulted from the shedding and secretion of plasma membrane and endomembranes, a normal activity for secretory mammary cells [Kinsella and McCarthy, Biochim. Biophys. Acta, 164:530-539 (1968)].

Acidification of Cellular Microsomes: The acidification of cellular microsomes was assayed spectrophotometrically. Two different approaches were used for assaying acidification: Acidification of the total microsomal preparation using quenching of acridine orange and acidification of the recycling endosomes by monitoring the fluorescence from a microsomal preparation from cells that had previously endocytosed FITC-transferrin.

To prepare the microsomes, cells were grown to confluence in 10 level Cell Factories (Nunc), trypsinized, washed three times with cold PBS and lysed with a Dounce homogenizer (Pestle A) in 0.25 M sucrose, 20 mM HERES (pH 7.4), 1 mM DTT, 1 mM EDTA, and 1×protease inhibitor mix (1 mg/ml leupeptin, 1 mg/ml pepstatin A, 1 mg/ml aprotinin, and 16 μM PMSF mixed to 100× before use). The homogenate was centrifuged twice for 10 minutes at 3000 g to remove unbroken cells and nuclei. The supernatant was layered over 20 ml of 0.5 M sucrose (20 mM HERES (pH 7.4), 1 mM DTT, 1 mM EDTA, 1×protease inhibitor mix) and 1 ml of 2 M sucrose and centrifuged for one hour at 100,000 g (Beckman Ti60 Rotor). Microsomes are collected at the 0.5 M and 2 M interface.

To monitor acidification of the total microsomal fraction, the quenching of acridine orange fluorescence was monitored essentially as described previously [Barasch et al., J. Cell Biol., 107:2137-2147 (1988); Barasch et al., Nature (London), 352:70-73 (1991)]. Acidic vesicles accumulate acridine orange to high concentrations resulting in the self quenching of the dye. This accumulation causes depletion of the acridine orange from the extra-vesicular space, and a decrease of the overall fluorescence of the sample. Fluorescence was measured on an SLM Aminco-Bowman series 2 luminescence spectrometer with a λex=490 nm and λem=530 nm. Microsomes (80 μg protein) were suspended in 2.5 ml vesicle buffer (125 mM KCl, 5 mM MgCl₂, 20 mM HEPES (pH 7.4), 1 mM DTT, 1 mM EDTA, 2 mM NaN3), with 6 μM acridine orange (5 mM stock in H₂O) in a cuvette. To examine the ability of vesicles to generate a ΔpH in the presence of tamoxifen or bafilomycin A1, 0, 1, 2, 4, 8 μM of tamoxifen (10 mM stock in EtOH) or 10 nM bafilomycin A1 (10 mM stock in 10% DMSO) was added. The sample was allowed to equilibrate by stirring for 30 minutes at 25° C. After 5 minutes of recording to establish baseline, 1 mM Tris-ATP was added to begin acidification (100 mM stock, titrated to pH 7.4 with 1 M Tris-base before use). Twenty minutes later, 2.5 μM of nigericin (10 mM stock in EtOH) was added to dissipate any ΔpH formation. To examine the effects of Tamoxifen and Bafilomycin A1 on vesicles with a pre-existing ΔpH, the procedure is identical to the above with the following exception: 10 minutes after the addition of Tris-ATP which resulted in partial acidification, Tamoxifen or Bafilomycin A1 were added.

To monitor acidification from the recycling endosomal fraction, cells were first incubated with FITC-transferrin for 30 minutes before lysis and isolation of microsomes. Acidification was monitored by excitation of the FITC fluorophore at 450 and 490 nm and measuring emission at 520 nm as described above.

Results

Cell Viability: Tamoxifen has been proposed to reverse drug resistance in MCF-7/ADR cells so the concentration at which Tamoxifen affects the sensitivity to Adriamycin was determined. Adriamycin intercalates in the DNA and inhibits topoisomerase II. MCF-7/ADR cells were exposed to several concentrations of the chemotherapeutic in the presence of 0, 5, or 10 μM Tamoxifen for a six-hour period, and then rinsed in drug-free medium. After three days cell viability was assayed.

In the absence of Tamoxifen, exposure to 1 μM Adriamycin resulted in less than 20% of the cell death (FIG. 18, ●). However, in the presence of 5 μM Tamoxifen, the same amount of Adriamycin caused 95% cell death (FIG. 18 ▪). Cells treated with 5 μM Tamoxifen, in the absence of Adriamycin, showed no decrease in viability (FIG. 18 ▪). Thus Tamoxifen at 5 μM had a significant effect on reversing the drug-resistance of the MCF-7/ADR cells. This is similar to what has been previously observed for monensin and nigericin [Schindler et al., Biochemistry, 35:2811-2817 (1996); Example 4, above].

Cellular localization of Adriamycin In MCF-7 and MCF-7/ADR cells: The cellular distribution of chemotherapeutics was assayed by following the drug Adriamycin. Adriamycin was chosen as the probe both because it is frequently used in treatment of breast cancer and because it is, like many other chemotherapeutics, a naturally fluorescent heterocyclic amine. Thus, its distribution can be visually followed in living cells. Adriamycin accumulation in MCF-7 and MCF-7/ADR cells reaches a steady-state distribution in approximately 60 minutes. In the drug-sensitive MCF-7 cells (FIG. 19A, bright-field image), Adriamycin is found diffusely through the nucleoplasm and cytoplasm (FIGS. 19B, 19C and [Schindler et al., Biochemistry, 35:2811-2817 (1996); Example 4, above]. In contrast, in the MCF-7/ADR cells (FIG. 19D, bright-field image), the Adriamycin is observed to be primarily in punctate cytoplasmic organelles (FIGS. 19E, F). This pattern of accumulation is similar to what has been observed in many other drug-resistant cells types [Willingham et al., Cancer Res., 46:5941-5946 (1986); Hindenburg et al., Cancer Res., 49:4607-4614 (1989); Weaver et al., Exp. Cell Res., 196:323-329 (1991); Lankelma et al., Biochim. Biophys. Acta Mol. Cell Res., 1093:147-152 (1991); Gervasoni, Jr., et al., Cancer Res., 51:4955-4963 (1991); Jaffrézou et al., Cancer Res., 52:6440-6446 (1992); Coley et al., Br. J. Cancer, 67:1316-1323 (1993); Rutherford and Willingham, Journal of Histochemistry and Cytochemistry, 41:1573-1577 (1993)]. The cytoplasmic organelles loaded with Adriamycin co-localize with the lysosomes, and the perinuclear compartments of the recycling endosomes and the trans-Golgi network. This co-localization was achieved by double labeling with Adriamycin and Lysosensor Blue DND-167 for the lysosomes, NBD-ceramide for the trans-Golgi network, and BODIPY transferrin for the recycling endosomes (Example 4, above).

Effect of reversers of MDR on Adriamycin distribution: Numerous agents have been observed to reverse drug-resistance in in vitro assays. Each of these agents sensitized MCF-7/ADR cells to Adriamycin in a manner similar to Tamoxifen (FIG. 18). The effects of these drugs on the distribution of Adriamycin in MCF-7/ADR cells were tested at the same concentrations at which they sensitized the cells to chemotherapeutics. MCF-7/ADR cells were incubated with Adriamycin and then the Adriamycin distribution was recorded after the addition of tamoxifen (0.2-10 μM) (FIG. 20B), cyclosporin (5 μM), and verapamil (10 μM). Treatment of MCF-7/ADR cells with tamoxifen (10 μM) resulted in a shift of Adriamycin from punctate perinuclear compartments (FIG. 20A) to the nucleoplasm and cytoplasm (FIG. 20B is the same field of cells as in FIG. 20A, but 30 minutes after addition of tamoxifen). The distribution of Adriamycin in these cells was similar to that observed in MCF-7 drug-sensitive cells in the absence of tamoxifen (FIG. 19), [Schindler et al, Biochemistry, 35:2811-2817 (1996); Example 4, above]. Similarly, treating MCF-7/ADR cells with verapamil or cyclosporin shifted Adriamycin from punctate cytoplasmic organelles to a diffuse cytoplasmic and nucleoplasmic distribution [Merlin et al., Cytometry, 20:315-323 (1995)]. These observations are consistent with a dispersal of the chemotherapeutic drugs from within the cytoplasmic organelles into the cytoplasm and nucleoplasm.

Cellular pH: Effect of tamoxifen on acridine orange labeling of MCF-7/ADR cells Adriamycin accumulates within the acidified organelles of MCF-7/ADR cells. Dissipation of the pH gradient in these organelles by protonophores or inhibitors of the H+-ATPase disperses the accumulated Adriamycin (Example 4, above). Since agents that reverse MDR also disperse Adriamycin from these organelles, effects of these MDR-reversing agents were tested on acidification of the organelles.

Acridine orange a weak-base fluorescent probe that accumulates in acidic compartments [Barasch et al., J. Cell Biol., 107:2137-2147 (1988)], was used to test for the presence of acidic organelles. When accumulated at a high concentration in these acidic compartments, acridine orange demonstrates a concentration-dependent long wavelength shift in the fluorescence emission. Thus, it appears as a “red” fluorescence within acidic cellular compartments and as a “green” fluorescence at lower concentration [Barasch et al., J. Cell Biol., 107:2137-2147 (1988)].

In MCF-7/ADR cells, as in non-transformed cells, acridine orange produced a red fluorescence in the perinuclear position (FIG. 21B). The addition of 10 μM tamoxifen produced a steady decrease of the red acridine orange fluorescence in MCF-7/ADR cells (FIG. 21C shows the same field of cells as in FIG. 21B). The decrease, indicating a diminished accumulation of acridine orange, was observed immediately upon addition of tamoxifen and persisted for the following 60 minutes. Similar effects were observed after addition of the protonophores monensin and nigericin or the blockers of the H+-ATPase such as Bafilomycin A1 or concanomycin A (Example 4, above). This is consistent with a tamoxifen-mediated effect on reducing acidification within the organelles of the cell. The acridine orange fluorescence in MCF-7/ADR cells treated with tamoxifen was similar to its fluorescence in MCF-7 drug-sensitive cells (FIG. 21A) that are abnormal in organelle acidification [Schindler et al., Biochemistry, 35:2811-2817 (1996); Example 4].

Effect of tamoxifen on acridine orange labeling of other cell types: Tamoxifen is a partial estrogen receptor agonist and the MCF-7 breast tumor line expresses the estrogen receptor. To determine whether tamoxifen's effect on organelle acidification is mediated by the estrogen receptor, an estrogen receptor negative, multidrug resistant breast cancer cell line, MDA-A1 [Ciocca et al., Cancer Res., 42:4256-4258 (1982); Taylor et al., Cancer Res., 44:1409-1414 (1984)], was tested. The acridine orange fluorescence of the MDA-A1 cells is shown in FIG. 22A. There is red fluorescence in punctate cytoplasmic organelles and in a perinuclear region. After a thirty minute incubation with tamoxifen, there was a substantial decrease in the red acridine orange fluorescence (FIG. 22C).

Both MCF-7/ADR and MDA-A1 cells express the P-glycoprotein (Pgp) [Fairchild et al., Cancer Res., 47:5141-5148 (1987a); Fairchild et al., Proc. Natl. Acad. Sci. USA, 84:7701-7705 (1987b)] which has been demonstrated to confer multidrug-resistance when transfected into cells [Debenham et al., Mol. Cell Biol., 2:881-889 (1982); Gros et al., Mol. Cell Biol., 6:3785-3790 (1986); Deuchars et al., Mol. Cell Biol., 7:718-724 (1987)]. To determine whether Pgp is required for tamoxifen's effect, CHO cells were used. CHO cells are often used for transfection studies with Pgp since endogenous levels of the protein are non-existent, or too low to be measured [Debenham et al., Mol. Cell Biol., 2:881-889 (1982)]. The acridine orange fluorescence for the cells is shown before (FIG. 22B) and 30′ after the addition of (FIG. 22D) 10 μM tamoxifen. Tamoxifen substantially reduced the red acridine orange fluorescence, and thus organelle acidification, in CHO cells. Similar results have been observed in freshly dissociated mouse tail fibroblasts cells. These data indicate that tamoxifen's effect on acidification is independent of both the estrogen receptor and Pgp.

Quantification of pH in specific organelles: Acridine orange is useful as a qualitative assay of organelle acidification. However, it cannot be used to quantify pH nor to selectively assay the pH in specific compartments. It primarily reports acidification in the lysosomes, the most acidic organelle in the cell. In addition, MDR reversers may affect acridine orange distribution not through pH but by inhibiting active transport of the probe into organelles or by non-pH dependent processes. To selectively probe and quantify the pH in different organelles, the pH-sensitive dyes SNARF and FITC were used. These dyes can be used to quantify pH and they can be conjugated to probes that can be selectively localized in specific organelles of the cell. FITC is a dual excitation probe where the pH is determined by the ratio of the emission intensity at 520 nm between excitation at 514 nm and excitation at 450 nm. SNARF is a dual emission probe where the pH is determined by the ratio of emission intensity at 570 nm and 630 nm when excited at 514 nm (see Materials and Methods, above).

pH in the recycling endocytic pathway: To selectively examine the pH in the recycling endocytic vesicles, the ratiometric pH probe FITC was conjugated to transferrin. Transferrin is endocytosed and recycled back to the cell surface through the recycling endosomes. It is not detected in the lysosomal pathway and is a selective marker for the recycling endocytic pathway [Dunn et al., J. Cell Biol., 109:3303-3314 (1989)] Example 4, above]. The pH in the recycling endosome compartment is 6.1 in MCF-7/ADR cells (Table 2). After addition of 10 μM tamoxifen, the pH shifts to 6.7. These results indicate that the endocytic pathway is one of the compartments whose pH was affected by tamoxifen treatment. The results from the pH probes conjugated to transferrin confirms that the effects of tamoxifen on acridine orange fluorescence are the consequence of a change of organelle pH.

TABLE 2

| | | | Recycling | Lysosomes | lysosomes |
|---|---|---|---|---|---|
| | Cytosol | Nucleus | endosomes | SNARF- | FITC |
| | (SNARF 70 kD | (SNARF 10 kD | FITC-conjugated | conjugated | conjugated |
| | dextran) | dextran) | transferrin | dextran | dextran |
| MCF-7/ADR | 7.10 ± 0.1 | 7.20 ± 0.1 | 6.1 ± 0.2 | <6.0♥ | 5.2 |
| + Tamoxifen | 7.20 ± 0.1 | 7.20 ± 0.1 | 6.7 ± 0.2 | 7.1 | >6.6 [symbol] |
| change with | +0.1 | 0.0 | +0.5 | >+1.1 | >+1.2 |
| Tamoxifen | | | | | |

♥: In these calibrations the SNARF fluorescence cannot discriminate between different pH values below 6.0 [symbol]
: In these calibrations the FITC fluorescence cannot discriminate between different pH values above 6.6

Lysosomal pH: To selectively label the lysosomes, cells were pulsed with FITC or SNARF conjugated to dextrans for one hour. Dextrans enter the cell through endocytosis and are sorted to the lysosomes where they remain. Following a one hour chase there is no remaining fluorescence in the endosomes. The pattern of dextran loading match that of the lysosomal dye LysoSensor Blue DND167 (Molecular Probes). The emission of the SNARF-dextran in the MCF-7/ADR cells indicated the pH was <6.0. After addition of 10 μM tamoxifen, the pH shifted to 7.1 (Table 2). The ratiometric calibration of SNARF is not very sensitive at pH below 6. Thus, the experiments were repeated using FITC conjugated to dextran. The pH reported by FITC-dextran in the MCF-7/ADR cells was 5.2±0.1. After incubation with 10 μM tamoxifen, the pH shifted to >6.6 (the calibration of FITC was not reliable above pH 6.6; Table 2).

To further confirm tamoxifen's effect on lysosomal pH, electron microscopic localization of DAMP was assessed. DAMP is weak base that accumulates in acidic organelles. Quantification of subcellular concentration can be determined by using anti-DNP antibodies and gold-conjugated secondary antibodies [Barasch et al., Nature (London), 352:70-73 (1991)]. In the MCF-7/ADR cells, the lysosomes were heavily labeled with gold antibodies demonstrating that they were acidic (FIG. 24A). The average density of gold particles was 7.02/μm² per lysosomal area. When MCF-7/ADR cells were treated with tamoxifen prior to incubation with DAMP, the anti-DNP labeling in the lysosomes was substantially reduced (FIG. 24B). In the presence of tamoxifen the average density was 2.0/μm² per lysosome area. Similarly, with monensin treatment, the concentration fell to zero/μm².

Cytosolic and nuclear pH: The MCF-7/ADR cells were loaded with SNARF-conjugated to either 10 kD or 70 kD dextrans [McNeil et al., J. Cell Biol., 98:1556-1564 (1984)]. The 70 kD dextrans remained exclusively cytosolic Example 4, above. In contrast, the 10 kD dextrans were found both in the cytoplasmic and nucleoplasmic compartment Example 4, above. The SNARF-conjugated dextrans are too large to cross membranes and thus are selective markers for the cytoplasmic and nucleoplasmic pH (rather than total cellular pH). Using this method, the cytoplasmic pH was observed to be 7.1±0.1 for the MCF-7/ADR cells (n=13) and MCF-7 mean pH 6.65+/−0.4 (n=16) (Example 4, above). The addition of 10 μM Tamoxifen shifted the cytosolic pH 0.1 units more alkaline (Table 2). The ionophore monensin, which also reverses drug-resistance [Schindler et al., Biochemistry, 35:2811-2817 (1996)], shifted the cytosolic pH 0.2 units more alkaline. Similarly, verapamil had no measurable effect on cytosolic pH. None of these agents had a measurable effect on nuclear pH.

In vitro Acidification of Vesicles: Total microsomal preparation: To determine whether tamoxifen affects organelle pH directly or indirectly, its effects on acidification of isolated microsomes of MCF-7/ADR cells was determined. Acridine orange was used as a probe for lumenal acidification [Barasch et al., J. Cell Biol., 107:2137-2147 (1988); Barasch et al., Nature (London), 352:70-73 (1991)]. As vesicles acidify, they accumulate acridine orange to concentrations that result in self-quenching of the fluorescent probe. This accumulation within vesicles partially depletes the extra-vesicular free acridine orange resulting in a decrease in total fluorescence.

Fluorescence microscopy of living cells indicated that tamoxifen inhibited acidification of all microscopically visible acidic organelles (e.g., TGN, endosomes, and lysosomes) (FIG. 21). Therefore, this first series of measurements utilized a total cellular microsomal fraction for the in vitro acidification assays. Acidification was initiated by the addition of ATP to a purified microsomal fraction in the absence of cytosol (FIG. 25A, at t=300 sec). Over the time course of the subsequent 1200 seconds, there was a reduction of the acridine orange fluorescence, suggesting an accumulation, and thus quenching, of acridine orange within the lumen of the microsomes (FIG. 25A). To test whether the reduction of acridine orange fluorescence was a consequence of acidification of the microsomes, nigericin (a potassium-proton ionophore) was added at the end of each reaction (t=1500 seconds). In all experiments the acridine orange fluorescence returned to its pre-ATP levels (FIG. 25A). This indicates that the decreased fluorescence was the consequence of the generation of a pH gradient.

When MCF-7/ADR vesicles were pre-treated with tamoxifen for 30 minutes, there was a dose-dependent inhibition of acridine orange quenching (FIG. 24A). Inhibition was noticeable with 1 μM tamoxifen and acidification was totally blocked with 8 μM tamoxifen. To quantify the effects of tamoxifen on acidification, acidification (as assayed by quenching of acridine orange fluorescence) was plotted as a function of tamoxifen concentration (FIG. 24B). The ID50 is approximately 3 μM. As expected, monensin also inhibited acidification in our assay. As a positive control, Bafilomycin A1 was employed, a potent and specific inhibitor of the vacuolar type H+-ATPase responsible for acidification of all intracellular compartments [Bowman et al., Proc. Natl. Acad. Sci. USA, 85:7972-7976 (1988)].

The time course of tamoxifen's effects on pH were examined to determined whether tamoxifen dissipated pH gradients (similar to nigericin by acting as a protonophore) or whether it only blocked further acidification (similar to Bafilomycin A1 by blocking the proton ATPase). Vesicles were allowed to acidify for 10 minutes prior to the addition of tamoxifen (FIG. 24C). ATP was added to initiate acidification (FIG. 24C). Addition of tamoxifen rapidly reversed acidification and caused an almost complete dissipation of the pH gradient within 5 minutes (FIG. 24C). Bafilomycin A1 dissipated the pH gradient at a much slower rate, even when added at 100 nM (ten times the concentration that blocked 95% of acidification). Two proton ionophores nigericin (K+/H+ exchanger, FIG. 24C) and monensin (Na+/H+ exchanger), dissipated the pH gradient significantly faster than tamoxifen. Tamoxifen has no effect on acridine orange fluorescence in the absence of membranes.

The in vitro acidification assay used purified microsomes that had been resuspended in a salt buffer without additional cytosolic or nuclear components. This indicated that the effects of tamoxifen on pH may be independent of cytosolic factors, such as the estrogen-receptor, and of transcription and protein synthesis. To determine whether the effect of tamoxifen on acidification is specific to the MCF-7/ADR drug-resistant breast cancer cell line, similar in vitro experiments were performed on fresh liver and kidney tissue from mice. The results obtained with these tissues were similar. Therefore, the effect of tamoxifen on organelle acidification appears to be a general phenomenon.

In vitro acidification of recycling endosomes: To selectively examine the acidification of endosomes in vitro, the MCF-7/ADR cells were incubated with FITC-transferrin before lysis and isolation of microsomes. This ensured that the only fluorescence signal was from the recycling endosomes since the FITC-transferrin was constrained to this pathway. Upon addition of ATP there was a decrease in the ratio of the FITC-emission (FIG. 24D). This signal was judged to be the consequence of acidification since it was reversed upon the addition of nigericin. The addition of 2.5 μM tamoxifen partially reversed the acidification in these organelles. This acidification was further reduced by raising the tamoxifen concentration an additional 2.5 μM. This indicates that the recycling endosomes are one of the compartments whose acidification in this in vitro assay is blocked by tamoxifen. The results from the FITC-transferrin are also an independent test of the results from the acridine orange assays of in vitro acidification (FIG. 23A, 23B, 23C). Since the fluorophore (FITC) is conjugated to transferrin, and unable to cross membranes, these results indicate that the effects of tamoxifen on acridine orange fluorescence occurs via pH in the lumen of the microsomes and not on the partitioning of acridine orange across membranes.

Rates of secretion from the recycling endocytic pathway: The PSS model predicts that either inhibition of organelle acidification and of secretion would increase chemotherapeutic drug sensitivity. In addition, reversers of MDR inhibit acidification (see above) and proper acidification has been reported to be important for normal transport of the recycling [Mellman et al., Annu. Rev. Biochem., 55:663-700 (1986); Maxfield and Yamashiro, In: Intracellular traficking of proteins, C. J. Steer and J. A. Hanover, eds., Cambridge: Cambridge University Press, pp. 157-182 (1991)] and biosynthetic [Basu et al., Cell., 24:493-502 (1981); Tartakoff, Cell, 32:1026-1028 (1983); Griffiths et al., J. Cell Biol., 96:835-850 (1983)] exocytotic pathways. Thus, the effect of reversers of MDR on the rate secretion was examined. Two different assays were used to measure transport to the surface from the endocytic pathway. Cells were loaded with the fluorescent probe BODIPY conjugated to transferrin or with the fluorescent probe NBD conjugated to the lipid sphingomyelin. Transferrin is selective for the fluid phase of the endocytic system. NBD-Sphingomyelin partitions into membrane systems, transiently accumulates in the Golgi before exocytosis, and thus is partially selective for the lipid phase of the biosynthetic pathway. The two probes were allowed to be endocytosed to a steady-state concentration. Their rate of transport back to the surface was then measured.

Fluid phase transport: Cells were loaded with BODIPY-transferrin for 20 minutes and transferred to dye free medium. Cell associated fluorescence was quantified 0, 5, 15, and 25 minutes later. In MCF-7/ADR cells, BODIPY-transferrin was chased out to 50% of its steady state level in 5 minutes (FIG. 25, solid line). In contrast, when the cells were treated with tamoxifen, the rate of recycling of the BODIPY-transferrin was substantially slower. In the presence of 10 μM tamoxifen it took 30 minutes for the concentration of intracellular BODIPY-transferrin to decrease to 50% of its steady-state level. The rate of transport of BODIPY-transferrin in MCF-7/ADR cells treated with tamoxifen was comparable from the rates of transport measured in the drug-sensitive MCF-7 cells. Thus the rate of transport of the transferrin receptor is slowed by treatment with tamoxifen. This could be the consequence of either a direct effect on the kinetics of vesicular transport through the recycling endosomal system or alternatively, it could reflect a pH-sensitive step in the sorting and transport specifically of the transferrin receptor.

Lipid Transport: NBD-ceramide has been previously used to monitor the rate of lipid transport through the endocytic system. Cells were incubated with NBD-ceramide which is taken up and converted into NBD-sphingomyelin. The NBD-sphingomyelin transiently accumulates within the Golgi. The transport of NBD-ceramide out of the cell was followed over two hours. Since the probe undergoes a red-shift in fluorescence when accumulated to high concentrations, the yellow in FIG. 26 indicate areas of high probe concentration. After two hours, less than 25% of the sphingomyelin remained associated with a field of cells (FIG. 26, left column, FIG. 27, solid black line). In the presence of tamoxifen (10 μM), more than 70% of the sphingomyelin fluorescence remained associated with the cells (FIG. 26, middle column, FIG. 27, triangles). This rate is similar to the speed with which the sphingomyelin is transported to the surface in the drug-sensitive MCF-7 cells.

Discussion

Cellular mechanisms for drug-resistance: It has been previously shown that agents that inhibit acidification of cytosolic organelles reverse Adriamycin resistance [Schindler et al., Biochemistry, 35:2811-2817 (1996) Example 4, above]. This report demonstrates that agents that reverse drug-resistance inhibit acidification of cytoplasmic organelles. From these data, the strongest conclusion that can be reached is that disrupting organelle acidification disrupts—either directly or indirectly—the mechanism(s) of drug-resistance. There are two potential explanations for these observations. One is the PSS hypothesis: preventing acidification blocks the protonation of drugs in the secretory pathway; this blocks sequestration of drugs into these organelles away from the cytosol and reduces exocytosis of drugs out of the cell. A second is the detoxification hypothesis: detoxification is a pH-dependent process within the lysosomes and/or endocytic or biosynthetic secretory pathways; blocking acidification blocks the detoxification. It is possible that both mechanisms contribute to the drug-resistance phenotype.

It has been previously suggested that drug resistance is a phenomenon that occurs at the plasma membrane, either through a drug-efflux pump [Gottesman et al., Annu. Rev. Genet., 29:607-649 (1995)] or as a consequence of a voltage or pH gradient dependent partitioning of chemotherapeutic drugs across the plasma membrane [Roepe et al., Biochemistry, 32:11042-11056 (1993); Roepe et al., Biochemistry, 33:11008-11015 (1994); Simon et al., Proc. Natl. Acad. Sci. USA, 91:1128-1132 (1994a); Simon and Schindler, Proc. Natl. Acad. Sci., USA, 91:3497-3504 (1994b)]. Experiments disclosed herein indicate that these mechanisms may not contribute significantly to Adriamycin-resistance in these cells. A wide variety of agents including ionophores (e.g., monensin, nigericin), calcium-channel blockers (e.g., verapamil), estrogen-receptor agonists (e.g., tamoxifen), and phosphatase inhibitors (e.g., cyclosporin A) all reverse acidification of cytoplasmic organelles at the concentrations at which they reverse drug resistance. Many of these agents have also been implicated as inhibitors of a drug-efflux pump. In order for the plasma membrane drug-efflux pump to be the dominant resistance mechanism, data from this report would necessitate a causative or correlative relationship between acidification of intracellular organelles and activity of this pump. In addition, at the concentrations sufficient to reverse drug resistance these agents do not significantly affect cytosolic pH. Thus, they are unlikely to have a pH mediated effect on processes associated with the plasma membrane. However, since each of these agents is membrane-permeable, care should be taken before eliminating some yet-to-be discovered common plasma membrane targets for these drugs.

Pleiotropic nature of drug resistance: A large body of literature has documented a multitude of functional and structural abnormalities in drug-sensitive tumor cells. These include: a) changes in the patterns of endocytosis and secretion [Sehested et al., Br. J. Cancer, 56:747-751 (1987); Sehested et al., Biochem. Pharmacol., 37:3305-3310 (1988); Schindler et al., Biochemstiry, 35:2811-2817 (1996)], b) modifications in the composition and dynamics of the plasma membrane [Escriba et al., Biochemistry, 29:7275-7282 (1990)], c) alterations in the expression of glycolipids, d) changes in the cytoplasmic pH [Keizer and Joenje, J. Natl.. Cancer Inst., 81:706-709 (1989); Thiebaut et al., Journal of Histochemistry and Cytochemistry, 38:685-690 (1990); Roepe et al., Biochemistry, 32:11042-11056 (1993); Roepe et al., Biochemistry, 33:11008-11015 (1994); Simon et al., Proc. Natl. Acad. Sci. USA, 91:1128-1132 (1994a); Simon and Schindler, Proc. Natl. Acad. Sci. USA, 91:3497-3504 (1994b); Example 4, above] changes in vesicular pH [Sehested et al., Biochem. Pharmacol., 37:3305-3310 (1988); Moriyama et al., J. Biochem. (Tokyo), 115:213-218 (1994); Rhodes et al., Br. J. Cancer, 70:60-66 (1994); Simon et al., Proc. Natl. Acad. Sci. USA, 91:1128-1132 (1994a); Simon and Schindler, Proc. Natl. Acad. Sci. USA, 91:3497-3504 (1994b); Schindler et al., Biochemistry, 35:2811-2817 (1996); Example 4, above] alterations in the architecture of the perinuclear recycling compartment (PRC) and trans-Golgi network (TGN) [Schindler et al., Biochemistry, 35:2811-2817 (1996)], g) differences in the glycosylation of plasma membrane proteins [Basrur et al., Oncology, 40:202-204 (1983); Basrur et al., Oncology, 42:328-331 (1985)], h) secretion of lysosomal enzymes in tumor cells but not drug-resistant cells [Vignon et al., J. Natl. Canc. Inst., 84:38-42 (1992)] and i) differences in attachment and migration of cells [Apostolopoulos and McKenzie, Critical Reviews in Immunology, 14:293-309 (1994)]. Many of these defects seem normalized in drug-resistant cell lines and has been termed “reverse transformation”. [Biedler and Spengler, Cancer & Metastasis Reviews, 13:191-207 (1994); Schindler et al., Biochemistry, 35:2811-2817 (1996); Example 4, above].

Each of these defects may be the consequence of a common mechanism resulting from an abnormal organelle pH. Acidification of cytoplasmic organelles has been shown to affect patterns and rates of endocytosis and secretion [Tartakoff and Vassalli, J. Cell Biol., 79:694-707 (1978); Tartakoff, Cell, 32:1026-1028 (1983); Cosson et al., J. Cell Biol., 108:377-387 (1989); Parczyk and Kondor-Koch, Eru. J. Cell Biol., 48:353-359 (1989); Mellman, Journal of Experimental Biology, 172:39-45 (1992); Sakaguchi et al., J. Cell Biol., 133:733-747 (1996)]. It is also known to affect glycosylation of membrane proteins and lipids [Keller et al., Biochim. Biophys. Acta, 566:266-273 (1979)] and secretion of lysosomal enzymes [Ledger et al., Biochemical & Biophysical Research Communications, 182:675-681 (1983)]. This is similar to what has been observed in CFTR-mutant cells [Barasch et al., Nature (London), 352:70-73 (1991)] and in vesicular acidification in mutant Chinese hamster ovary (CHO) lines [Robbins et al., J. Cell Biol., 99:1296-1308 (1984); Roff et al., J. Cell Biol., 103:2283-2297 (1986)]. CHO cells that were selected for resistance to diphtheria toxin had abnormalities similar to those of the drug-sensitive tumor cells: abnormal ATP-dependent endosomal acidification [Robbins et al., J. Cell Biol., 99:1296-1308 (1984); Roff et al., J. Cell Biol., 103:2283-2297 (1986)]; decreased or altered sialylation of secreted proteins [Robbins et al., J. Cell Biol., 99:1296-1308 (1984); Roff et al., J. Cell Biol., 103:2283-2297 (1986)], and aberrant secretion of lysosomal hydrolases [Vignon et al., J. Natl. Canc. Inst., 84:38-42 (1992)]. It was suggested that the mutation responsible for the defects observed in the mutant CHO cells was an aberrant H+-ATPase or anion channel that was localized to both Golgi and endosomal compartments [Robbins et al., J. Cell Biol., 99:1296-1308 (1984); Roff et al., J. Cell Biol., 103:2283-2297 (1986)]. Similarly, abnormalities in the membrane transport of Cl− in CFTR mutant cells were related to decreased sialylation, defects in acidification of the TGN and endosomes and defective iron dissociation from transferrin, delayed endosomal/lysosomal ligand transfer, the loss of cAMP-stimulated exocytosis, and to aberrant regulation of membrane recycling [Bae and Verkman, Nature (London), 348:637-639 (1990); Barasch et al., Nature (London), 352:70-73 (1991); Bradbury et al., Science, 256:530-532 (1992)]. In these investigations, defective or unregulated ATP binding proteins (H+-ATPase) and/or Cl− channel(s) in endosomes and the TGN were shown to be responsible for decreased acidification of intracellular recycling and secretory compartments. Many of the reported phenotypic changes for the mutant CHO and cystic fibrotic cells have previously been observed following the treatment of cells with monensin, an ionophore that abrogates the acidification of the TGN, disrupts its organization, and inhibits the transit of membrane vesicles from the TGN to the plasma membrane [Basu et al., Cell., 24:493-502 (1981); Tartakoff, Cell, 32:1026-1028 (1983); Griffiths et al., J. Cell Biol., 96:835-850 (1983)].

A common parameter linking all these abnormal cell functions is a defect in the acidification of recycling and biosynthetic secretory compartments [Basu et al., Cell., 24:493-502 (1981); Tartakoff, Cell, 32:1026-1028 (1983); Griffiths et al., J. Cell Biol., 96:835-850 (1983); Robbins et al., J. Cell Biol., 99:1296-1308 (1984); Roff et al., J. Cell Biol., 103:2283-2297 (1986); Bae and Verkman, Nature (London), 348:637-639 (1990); Barasch et al., Nature (London), 352:70-73 (1991); Bradbury et al., Science, 256:530-532 (1992)]. The inability to establish a significant pH gradient between the cytoplasm and lumenal compartments of the recycling and secretory pathways is known to disrupt sialylation of proteins and lipids [Keller et al., Biochim. Biophys. Acta, 566:266-273 (1979); Eppler et al., Biochim. Biophys. Acta, 619:318-331 (1980); Barasch and Al-Awqati, J. Cell Sci. 106 Suppl., 17:229-233 (1993)], as well as sorting from TGN to endosomes, and sorting within the endocytic pathway. The resulting cellular perturbations in plasma membrane organization, cholesterol and lipid metabolism, mitochondrial function, and glycoprotein/glycolipid biosynthesis and targeting could account for the full spectrum of pleiotropic defects observed in drug-sensitive cancer cells. Repairing this acidification defect could result in the normalization of these pathways and result in cells capable of sequestering and secreting cytotoxic drugs through the normal activity of the recycling and/or secretory pathways.

Biochemical mechanism: A tremendous diversity has been reported both in the proteins that are believed to effect drug resistance and in the kinds of molecules that can reverse this resistance. MDR has been associated with changes in the expression of a number of proteins including three members of the ATP-binding cassette family of proteins (P-glycoprotein, multidrug-resistance associated protein, and a 100 kD protein), glutathione S-transferase π [Harris and Hochhauser, Acta Oncol., 31:205-213 (1992); Efferth and Volm, Cancer Lett., 70:197-202 (1993); Volm and Mattern, Onkologie, 16:189-194 (1993); De la Torre et al., Anticancer Res., 13:1425-1430(1993); Ripple etal., J. Urol., 150:209-214(1993)], catalase [Keizer et al., Cancer Res., 48:4493-4497(1988); Volm and Mattern, Onkologie, 16:189-194 (1993)], thymidylate synthase metallothionein [Volm and Mattern, Onkologie, 16:189-194 (1993)] as well as a subunit of the vacuolar H+-ATPase [Ma and Center, Biochemical and Biophysical Research Communications, 182:675-681 (1992)]. In contrast, topoisomerase II expression appears to be down regulated [Beck, J. Natl. Cancer Inst., 81:1683-1685 (1989); Beck, Cancer Treat. Rev. 17 Suppl A, 11-20 (1990); Friche et al., Cancer Res., 51:4213-4218 (1991)]. Agents that can reverse drug-resistance include calcium channel blockers (e.g., verapamil, nifedipine), phosphatase inhibitors (e.g., cyclosporin A, FK506), estrogen-receptor antagonists (e.g., tamoxifen), and blockers of neurotransmitter uptake (e.g., reserpine, yohimbine).

Despite their diversity, the above-described chemicals share many features. Changes in the cellular distribution of chemotherapeutics are fairly common to all drug-resistant cells: in all cases there is a shift from a diffuse, or slightly nuclear, distribution of Adriamycin to a peripheral distribution in punctate cytoplasmic organelles. Likewise, agents that reverse drug-resistance have shared features. At the concentration at which each drug reverses the drug-resistance phenotype, it also (a) reverses the pH profile of the drug-resistant cells to the pH profile of the drug-sensitive cells (FIG. 21) and (b) reverses the drug distribution phenotype of drug-resistant cells (FIG. 19) to the distribution observed in drug-sensitive cells. Each of the drugs that reverses drug-resistance has other specific cellular actions at lower concentrations. For example, the concentrations at which nifedipine and verapamil block calcium channels, tamoxifen binds the estrogen receptor, and cyclosporin A inhibits phosphatase, are too low to effect drug-resistance. Only concentrations that reverse acidification of cytoplasmic organelles are sufficient to reverse drug resistance.

The means by which a particular tumor cell loses its acidification may be a clue to what mechanisms have to be restored to regain its acidification. Loss of acidification could be the consequence of a defective H+-ATPase, loss of a counter-ion transport, changes in cytosolic proteins or in factors that modify the activity of H+-ATPase or counter ion transport, or an indirect effect of changes in cytosolic pH. Some drug-resistant cells lines over express a subunit of the proton-ATPase. The original acidification defect could be due to reduced activity of the H+-ATPase. Other MDR-cells over express the MRP, a protein implicated as a K+-channel. The defective acidification in this case could occur as a consequence of the loss of a counter-ion transport. These issues can be addressed with in vitro assays of acidification in different tumor cell lines.

Evolutionary and clinical considerations: Many toxins in the environment are alkaloids: weak bases. Acidification is observed in almost all eukaryotic secretory organelles from yeast to human. This acidification may have evolved, in part, as a defense mechanism. The toxins were protonated and sequestrated within the cells' organelles and then secreted. It is striking that all eukaryotic cells, except for these tumor cells, acidify the cytoplasmic organelles of the lysosomes, trans-Golgi network, perinuclear recycling compartment and secretory vesicles.

Some enzymatic processes have evolved to function optimally at the acidic pH found in the secretory pathway. These processes include sorting of proteins to the lysosome via the mannose-6-phosphate receptor and addition of sialic acids via the sialyltransferase. Cells that fail to acidify their secretory organelles are less successful both at adding sialic aids and at sorting enzymes to the lysosomes. Thus, any cell that fails to acidify its organelles is predicted to have the following four properties: reduced adhesion to the environment, disrupted cell-contact inhibition, secretion of lysosomal enyzmes, and increased sensitivity to chemotherapeutic drugs. All four properties are a consequence of aberrant acidification in the secretory pathway and all four are characteristics of metastatic tumor cells.

Cell adhesion: Failure to acidify in the TGN reduces the efficiency of the sialyl-transferase in the TGN. In mammary tissue the pH optimum of the α2-6 sialytransferase is 5.5 [Keller et al., Biochim. Biophys. Acta, 566:266-273 (1979)]. This is seen as reduced sialylation of cell surface proteins [Barasch and Al-Awqati, J. Cell Sci. 106 Suppl., 17:229-233 (1993); Robbins et al., J. Cell Biol, 99:1296-1308(1984); Roff et al., J. Cell Biol., 103:2283-2297 (1986); Schindler et al., Biochemistry, 35:2811-2817 (1996)]. Thus, surface recognition molecules such as integrins and selectins would be expected to have fewer sialic acids on their carbohydrate chains. It has been demonstrated that either modifying or reducing the sialic acids on these proteins reduces their adhesion to the basal lamina [Diacovso et al., Journal of Experimental Medicine, 183:1193-1203 (1996); Puri and Springer, J. Biol. Chem., 271:5404-5413 (1996)].

Contact inhibition: Recognition of cell-to-cell contact and the resulting inhibition of growth is mediated via cell adhesion molecules. There are a number of critical sialic acids in a joint region of the molecule. Interference with the sialylation of the cell adhesion molecules interferes with their interactions with neighboring cells and substrates [Weiss, J. Natl. Canc. Inst., 50:3-19 (1973); Cunningham et al., Proc. Natl. Acad. Sci. USA, 80:3116-3120 (1983); Hoffman and Edelman, Advances in Experimental Medicine and Biology, 181:147-160 (1984)]. Secretion of lysosomal enzymes: The mannose-6-phosphate receptor which recycles between the TGN and lysosome requires a vectorial pH gradient in order to sort protein cargo from the TGN to the lysosome. In the absence of the acidification, the mannose-6-phosphate receptor is less efficient at sorting, resulting in secretion of lysosomal enzymes. These enzymes contribute to catalysis of the basement membrane. Failure to acidify cellular organelles could promote metastatic behavior by: secretion of lysosomal enzymes; reduction of adhesion to the basal lamina; reduced recognition growth-inhibition by cell-cell contact. Fourth, the loss of organelle acidification results in the loss of a mechanism for sequestering alkaloids away from the cytosol, thus increasing sensitivity to environmental toxins, including chemotherapeutic drugs. When a patient is challenged with chemotherapy, tumor cells lacking organelle acidification would be more sensitive than non-transformed cells of the body. Tumor cells that restore acidification of the secretory pathway, MDR cells, also have restored sialylation, reduced secretion of lysosomal enzymes and, thus, are more like normal cells. Thus, it is not surprising that they have also undergone a “reverse transformation” with reduced metastatic behavior [Biedler and Spengler, Cancer & Metastasis Reviews, 13:191-207 (1994)].

Most breast cancers are dependent on estrogen for growth. Therefore, tamoxifen is often included in the chemotherapy regime for treatment [Jaiyesimi et al., Journal of Clinical Oncology, 13:513-529 (1995)]. Estrogen receptors are also believed to play an important role in the pathogenesis of breast cancer. High levels of estrogen exposure (e.g., obesity, early menarche, late menopause, late first child-bearing age) are major risk factors for its development. Thus, tamoxifen has been used by itself in numerous clinical studies as a prophylactic agent against breast cancer [Henderson et al., Science, 259:633-638 (1993); Jordan, Proceedings of the Society for Experimenetal Biology & Medicine, 208:144-149 (1995)]. Paradoxically, since tamoxifen is a partial agonist, it actually has many pro-estrogenic effects in menopausal women. It is used to slow the development of osteoporosis and atherosclerotic heart disease and alleviate many symptoms of menopause [Marchant, Cancer, 74:512-517 (1994)]. Many of its side effects, including increased risk for thrombotic events, endometrial cancer, liver disease and cancer, are also believed to stem from its pro-estrogenic nature. Because of these possible serious side effects, many uses of tamoxifen have been controversial [Jordan, Proceedings of the Society for Experimental Biology & Medicine, 208:144-149 (1995)]. Tamoxifen has been shown to have effects that are independent of the estrogen receptor. It has been reported to reverse MDR in vitro and in vivo in both estrogen receptor positive and estrogen receptor negative cells [Ramu et al., Cancer Res., 44:4392-4395 (1984); Chatterjee and Harris, British Journal of Cancer, 62:712-717 (1990); Pommerenke et al., Cancer Letters, 55:17-23 (1990); Berman et al., Blood, 77:818-825 (1991); Hu et al., European Journal of Cancer, 27:773-777 (1991); Trump et al., J. Natl. Canc. Inst., 84:1811-1816 (1992); Kirk et al., Biochem. Pharmacol., 48:277-285 (1994)]. There is evidence that it can inhibit the action of protein kinase C [O'Brian et al., J. Natl. Canc. Inst., 76:1243-1246 (1986)] and calmodulin-dependent cAMP phosphodiesterase [Lam, Biochemical & Biophysical Research Communications, 118:27-32 (1984)] and that it can induce normal cells surrounding the cancers to secrete the growth inhibiting cytokine transforming growth factor-b [Butta et al., Cancer Res., 52:4261-4264 (1992)]. There are also reports that Tamoxifen can modulate membrane fluidity, has antioxidant effects [Wiseman, Trends in Pharmacological Sciences, 15:83-89 (1994)] and blocks volume-activated chloride channels [Zhang et al., Journal of Clinical Investigation, 94:1690-1697 (1994)].

Tamoxifen has been introduced for the treatment of breast cancer largely because of its effects as a blocker of the estrogen receptor [Bush and Helzlsouer, Epidemiol. Rev., 15:233-243 (1993); Jordan, Br. J. Pharmacol., 110:507-517 (1993)]. However, our results demonstrate that at concentrations used to sensitize breast tumors to chemotherapeutics (0.5 μM-10 μM) Tamoxifen can also have a powerful effect directly on the acidification of cellular organelles and on transport through the secretory pathway—effects that are independent of estrogen-receptors and of any potential effects of Tamoxifen on transcription and protein synthesis. These observations should provide a cautionary note for the use of Tamoxifen in broad clinical studies. Treatments that modify this secretion dependent mechanism of drug resistance also impact on the ability of this pathway to cleanse the cell of mutagenic drugs or environmental carcinogens. Cells exposed to Tamoxifen may be more susceptible to environmental toxins and mutagens. A number of studies have demonstrated increased rates of tumors, especially of the endometrium and liver, in both humans and mice treated with Tamoxifen. Other studies suggest that Tamoxifen may enhance the effects of the carcinogen diethylnitrosamine [Dragan et al., Carcinogenesis, 16:2733-2741(1995)].

The results disclosed herein indicate that Tamoxifen reduces the organelle sequestration of chemotherapeutics resulting in a higher effective concentration of these toxins in the nucleus. Thus, it is an effective agent to sensitize drug-resistant cells to chemotherapeutics, which are environmental toxins. It may be prudent to screen for other blockers of the estrogen-receptor that do not also affect acidification of the secretory pathway before Tamoxifen becomes a widespread prophylactic for the prevention of breast cancer.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all base sizes or amino acid sizes, and all molecular weight or molecular mass values, given for nucleic acids or polypeptides are approximate, and are provided for the description.

The following is a listing of certain of the publications referred to numerically or in abbreviated fashion in the foregoing specification.

1. Ma, L. & Center, M. S. (1992) Biochem. Biophys. Res. Commun. 182, 675-681.

2. Cole, S. P. C., Bhardwaj, G., Gerlach, J. H., Mackie, J. E., Grant, C. E., Almquist, K. C., Stewart, A. J., Kurz, E. U., Duncan, A. M. V. & Deeley, R. G. (1992) Science 258, 1650-1654.

3. Gottesman, M. M. & Pastan, I. (1993) Annu. Rev. Biochem. 62, 385-427.

4. Dano, K. (1973) Biochim. Biophys. Acta 323, 466-483.

5. Higgins, C. F. & Gottesman, M. M. (1992) TIBS 17, 18-21.

6. Di Marco, A., Casazza, A. M., Dasdia, T., Necco, A., Pratesi, G., Rivolta, P., Velcich, A., Zaccara, A. & Zunino, F. (1977) Chem. Biol. Interact. 19, 291-302.

7. Owellen, R. J., Donigian, D. W., Hartke, C. A. & Hains, F. O. (1977) Biochem. Pharm. 26, 1213-1219.

8. Skovsgaard, T. (1977) Biochem. Pharm. 26, 215-222.

9. Warburg, O. (1956) Science 123, 309-314.

10. Thiebaut, F., Currier, S. J., Whitaker, J., Haugland, R. P., Gottesman, M. M., Pastan, I. & Willingham, M. C. (1990) J. Histochem. Cytochem. 38, 685-690.

11. Zunino, F., Di Marco, A. & Zaccara, A. (1979) Chem. Biol. Interact. 24, 217-225.

12. Zunino, F., Gambetta, R., Di Marco, A., Velcich, A., Zaccara, A., Quadrifoglio, F. & Crescenzi, V. (1977) Biochim. Biophys. Acta 476, 38-46.

13. Zunino, F., Gambetta, R., Di Marco, A. & Zaccara, A. (1972) Biochim. Biophys. Acta 277, 489-498.

14. Di Marco, A., Silvestrini, R., Di Marco, S. & Dasdia, T. (1965) J. Cell Biol. 27, 545-550.

15. Calendi, E., Di Marco, A., Reggiani, M., Scarpinato, B. & Valentini, L. (1965) Biochim. Biophys. Acta 103, 25-49.

16. Doskocil, J. & Fric, I. (1973) FEBS Letters 37, 55-58.

17. Weisenberg, R. C. & Timasheff, S. N. (1970) Biochemistry 9, 4110-4116.

18. Na, C. & Timasheff, S. N. (1977) Archives of Biochemistry and Biophysics 182, 147-154.

19. Weaver, J. L., Pine, P. S., Aszalos, A., Schoenlein, P. V., Currier, S. J., Padmanabhan, R. & Gottesman, M. M. (1991) Exp. Cell Res. 196, 323-329.

20. Boron, W. F. (1986) Annu. Rev. Physiol. 48, 377-388.

21. Lin, P., Ahluwalia, M. & Gruenstein, E. (1990) Am. J. Physiol. 258, C132-C139.

22. Gillies, R. J., Martinez-Zaguilan, R., Martinez, G. M., Serrano, R. & Perona, R. (1990) Proc. Natl. Acad. Sci. USA 87, 7414-7418.

23. Dalmark, M. & Storm, H. H. (1981) The Journal of General Physiology 78, 349-364.

24. Mayer, L. D., Bally, M. B. & Cullis, P. R. (1986) Biochim. Biophys. Acta 857, 123-126.

25. Keizer, H. G. & Joenje, H. (1989) J. Natl. Cancer Inst. 81, 706-709.

26. Nygren, P., Larsson, R., Gruber, A., Peterson, C. & Bergh, J. (1991) Br. J. Cancer 64, 1011-1018.

27. Gruber, A., Briese, B., Arestrom, I., Vitols, S., Björkholm, M. & Peterson, C. (1993) Leuk. Res. 17, 353-358.

28. Ling, V. & Thompson, L. H. (1974) J. Cell. Physiol. 83, 103-116.

29. Skovsgaard, T. (1978) Cancer Res. 38, 1785-1791.

30. Marsh, W., Sicheri, D. & Center, M. S. (1986) Canc. Res. 46, 4053-4057.

31. Inaba, M. & Sakurai, Y. (1979) Cancer Lett. 8, 111-115.

32. Ramu, A., Pollard, H. B. & Rosario, L. M. (1989) Int. J. Cancer 44, 539-547.

33. Sirotnak, F. M., Yang, C.-H., Mines, L. S., Oribé, E. & Biedler, J. L. (1986) J. Cell. Physiol. 126, 266-274.

34. Fojo, A., Akiyama, S., Gottesman, M. M. & Pastan, I. (1985) Cancer Res. 45, 3002-3007.

35. Carlsen, S. A., Till, J. E. & Ling, V. (1976) Biochim. Biophys. Acta 455, 900-912.

36. Beck, W. T., Cirtain, M. C. & Lefko, J. L. (1983) Mol. Pharmacol. 24, 485-492.

37. Epand, R. F., Epand, R. M., Gupta, R. S. & Cragoe, E. J., Jr. (1991) Br. J. Cancer 63, 247-251.

38. Mukhopadhyay, K., Parrack, P. K. & Bhattacharyya, B. (1990) Biochemistry 29, 6845-6850.

39. Wilson, L. (1970) Biochemistry 9, 4999-5007.

40. Beck, W. T. (1987) Biochem. Pharm. 36, 2879-2887.

41. Sehested, M., Skovsgaard, T., van Deurs, B. & Winther-Nielsen, H. (1987) J. Natl. Cancer Inst. 78, 171-179.

42. Sehested, M., Skovsgaard, T., van Deurs, B. & Winther-Nielsen, H. (1987) Br. J. Cancer 56, 747-751.

43. van Adelsberg, J. & Al-Awqati, Q. (1986) J. Cell Biol. 102, 1638-1645.

44. Hager, A., Debus, G., Edel, H.-G., Stransky, H. & Serrano, R. (1991) Planta 185, 527-537.

45. Mellman, I., Fuchs, R. & Helenius, A., Ann. Rev. Biochem, 55, 663-700 (1986).

46. Maxfield, F. R. & Yasmashiro, D. J. In Intracellular trafficking of proteins (eds Steer, C. J. & Hanover, J. A.) 157-182 (Cambridge University Press, Cambridge, 1991).

47. vanDeurs, B., Petersen, O. W., Olsnes, S. & Sandvig, K. International Review of Cytology, 117, 131-177 (1989).

48. Tartakoff, A. M. Cell 32, 1026-1028 (1983).

49. Simon, S. M. & Schindler, M. Proc. Natl. Acad. Sci USA 91, 3497-3504 (1994).

50. Whitaker, J. E., Haugland, R. P. & Prendergast, F. G. Analytical Biochemistry 194, 330-344 (1991).

51. Barasch, J., Kiss, B. Prince, A., Saiman, L., Gruenert, D. & Al-Awqati, Q. Nature (London) 352, 70-73 (1991).

52. Simon, S. M., Roy, D. & Schindler, M. Proc. Natl. Acad. Sci. USA 91, 1128-1132 (1994).

53. Van Dyke, R. & Belcher, J. D. Am. J. Physiol. Cell Physiol. 266, C81-C94 (1994).

54. Russell, J. T. & Holz, R. W. J. Biol. Chem. 256, 5950-5953 (1981).

55. Pagano, R. E., Martin, O. C., Kang, H. C. & Haughland, R. P. Journal of Cell Biology 113, 1267-1279 (1991).

56. Lippincott-Schwartz, J., Yuan, L. C., Tipper, C. Amherdt, M. Orci, L. & Klausner, R. D. Cell 67 601-616 (1991).

57. Al-Awqati, Q., Barasch, J. & Landry, D. J. Exp. Biol. 172 245-266 (1992).

58. Valverde, M. A., Diaz, M. Speulveda, F. V., Gill, D. R. Hyde, S. C. & Higgins, C. F. Nature (London) 355, 830-833 (1992).

59. Willingham, M. C., Richert, N. D., Corwell, M. M. et al. J. Histochem. Cytochem 35, 1451-1456 (1987).

60. Molinari, A., Cianfriglia, M., Meschini, S., Calcabrini, A. & Arancia, G. Int. J. Cancer 59, 789-795 (1994).

61. Labarca, C. & Paigne, K. Analytical Biochemistry 102, 344-352 (1980).

62. Schindler, M. et al J. Biol. Chem., manuscript submitted.

63. Berman, E. et al Blood 77, 818 (1991).

64. Chattedjee, M. et al British J. Cancer 62, 712 (1990).

65. Hamilto, G. et al Anticancer Res. 13, 2059 (1993).

66. Kirk, J. et al Biochem. Pharmacol. 48, 277 (1994).

67. Trump, D. L. et al J. Natl. Canc. Inst. 84, 1811 (1992).

68. Barasch, J. et al Nature 352, 70 (1991).

69. Lucocq, J. J. Cell Sci. 103, 875 (1992).

70. Horn, M. and Banting, G. Biochem. J. 301, 69 (1994).

71. Koval, M. and Pagano, R. E. J. Cell Biol. 108, 2169 (1989).

72. Mayor, S. et al J. Cell Biol. 121, 1257 (1993).

73. McGraw, T. E. et al J. Cell Physiol. 155, 579 (1993).

74. Zhang, J. J. et al J. Clin. Invest. 94, 1690 (1994).

75. Ehring, G. R. et al J. Gen. Physiol. 104, 1129 (1994).

Various publications are cited herein, the disclosure of which are incorporated by reference in their entireties. The citation of any reference herein should not be construed as an admission that such reference is available as “Prior Art” to the instant application.

What is claimed is:

1. A method for measuring the development or onset of multidrug resistance in a tumor cell in which such multidrug resistance is suspected, comprising determining whether there is a defect in the vesicular transport mechanism of an intracellular vesicular compartment of the cell; wherein said defect is symptomatic of the tumor cell being drug-sensitive; and wherein the absence of said defect is indicative of the onset or development of multidrug resistance in the tumor cell.

2. The method of claim 1 wherein the intracellular compartment of the cell is a secretory compartment.

3. The method of claim 2 wherein the secretory compartment is selected from the group consisting of a perinuclear recycling compartment (PRC), a recycling endosome, a secretory vesicle and the trans-Golgi network (TGN).

4. The method of claim 1 wherein determining whether there is a defect in the vesicular transport mechanism is performed by measuring the transport of a marker from the intracellular vesicular compartment to the exterior of the cell or the cell surface.

5. The method of claim 4 wherein the marker is a labeled protein.

6. The method of claim 5 wherein the labeled protein is labeled transferrin.

7. The method of claim 4 wherein the marker is a labeled lipid.

8. The method of claim 7 wherein the labeled lipid is labeled sphingomyelin.

9. The method of claim 4 wherein the marker is capable of being measured by a means selected from the group consisting of spectrophotometrically, spectrofluorometrically, by luminescence, by reflectance, by electron microscopy, and by radioactivity.

10. The method of claim 9 wherein the marker is capable of being measured spectrofluorometrically, and wherein the marker is measured by fluorescence microscopy.

11. The method of claim 9 wherein the marker is capable of being measured spectrofluorometrically, and wherein the marker is measured by confocal microscopy.

12. The method of claim 4 wherein the marker is capable of being measured through a biological activity, and wherein the biological activity is measured by a means selected from the group consisting of determining the activity on the surface of the cell, determining the activity on the outside of the cell, and determining the activity from the inside of the cell.

13. A method for screening potential drugs to treat multidrug resistant by identifying a candidate drug that decreases vesicular transport in a multidrug resistant tumor cell comprising:
(a) contacting a mammalian multidrug resistant tumor cell with a potential drug; wherein the multidrug resistant cell comprises an intracellular vesicular compartment that contains a marker; and
(b) measuring the transport of the marker from the intracellular vesicular compartment; wherein a potential drug is identified as a candidate drug if the transport of the marker from the intracellular vesicular compartment of the multidrug resistant tumor cell decreases.

14. The method of claim 13 wherein a plurality of potential drugs are tested at a plurality of drug concentrations.

15. The method of claim 13 wherein measuring the transport of the marker from the intracellular vesicular compartment is performed by measuring the rate of transport of the marker from the intracellular compartment of the cell to the exterior of the cell or the cell surface.

16. The method of claim 15 wherein the marker is a labeled protein.

17. The method of claim 16 wherein the labeled protein is labeled transferrin.

18. The method of claim 15 wherein the marker is a labeled lipid.

19. The method of claim 18 wherein the labeled lipid is labeled sphingomyelin.

20. The method of claim 15 wherein the marker is capable of being measured by a means selected from the group consisting of spectrophotometrically, spectrofluorometrically, by luminescence, and by radioactivity.

21. The method of claim 20 wherein the marker is capable of being measured spectrofluorometrically, and wherein the marker is measured by fluorescence microscopy.

22. The method of claim 20 wherein the marker is capable of being measured spectrofluorometrically, and wherein the marker is measured by confocal microscopy.

23. The method of claim 20 wherein the marker is capable of being measured through a biological activity, and wherein the biological activity is measured by a means selected from the group consisting of determining the activity on the surface of the cell, determining the activity on the outside of the cell, and determining the activity in the intracellular vesicular compartment.

24. An assay system for screening a potential drug for the treatment of multidrug resistance (MDR) comprising:
(a) a mammalian multidrug resistant tumor cell; and
(b) a labeled marker that can be used to measure the transport of the marker to the cell surface from the intracellular compartment of the cell.

25. A method for treating multidrug resistance in a mammal containing a multidrug resistant tumor cell comprising administering to the mammal a drug that decreases the rate of transport of an intracellular vesicular compartment of the multidrug resistant tumor cell in an amount effective to decrease the rate of transport and therein increase the drug sensitivity of the tumor cell.

26. The method of claim 25 wherein the drug is administered in association with the administration of a chemotherapeutic agent already under administration to the tumor cell.

27. The method of claim 26 wherein the drug is administered simultaneously with said chemotherapeutic agent.

28. The method of claim 26 wherein the drug is administered in a pharmaceutical composition comprising the drug and said chemotherapeutic agent.

29. The method of claim 25 wherein the drug is administered parenterally.

30. The method of claim 25 wherein the drug is administered orally.

31. A therapeutic composition for the treatment of multidrug resistance in a mammal comprising, in unit dose form, a drug that decreases the rate of transport of an intracellular vesicular compartment of said multidrug resistant tumor cell and a pharmaceutically acceptable excipient.

32. The composition of claim 31 wherein the composition includes a chemotherapeutic agent to which the mammal has developed said multidrug resistance.

33. A method for measuring the development or onset of pH-dependent multidrug resistance in a tumor cell in which such multidrug resistance is suspected, comprising determining whether there is a defect in the acidification of an intracellular vesicular compartment of the cell; wherein said defect is symptomatic of the tumor cell being drug-sensitive; and wherein the absence of said defect is indicative of the onset or development of multidrug resistance in the tumor cell.

34. The method of claim 33 wherein the intracellular compartment of the cell is a secretory compartment.

35. The method of claim 33 wherein determining whether there is there is a defect in the acidification of an intracellular vesicular compartment of the cell is performed by determining a measure of the pH of the intracellular vesicular compartment.

36. The method of claim 35 wherein the measure of the pH is determined by directly measuring the pH in the intracellular vesicular compartment.

37. The method of claim 36 wherein the pH is measured with a pH sensitive probe.

38. The method of claim 37 wherein the pH probe is targeted for a specific intracellular vesicular compartment.

39. The method of claim 38 wherein the pH probe is targeted to the endosomes by being associated with transferrin.

40. The method of claim 38 wherein the pH probe is targeted to the Golgi by being associated with verotoxin.

41. The method of claim 35 wherein the measure of the pH is determined indirectly by assaying for a detectable consequence of a defect in the acidification of an intracellular vesicular compartment.

42. The method of claim 41 wherein the consequence is selected from the group consisting of a decrease in the glycosylation of lipids or proteins on the surface of the cell, and an increase in the secretion of lysosomal enzymes from the cell.

43. The method of claim 42 wherein the decrease in the glycosylation of the lipids or proteins on the surface of the cell is identified by a decrease of sialic acids attached to lipids or proteins.

44. The method of claim 35 wherein the intracellular vesicular compartment of the tumor cell is infiltrated with a pH indicator prior to determining the pH.

45. The method of claim 44 wherein the pH indicator is selected from the group consisting of acridine orange, LysoSensor Blue DND-167, SNARF, SNAFL, FITC, DAMP, and BCECF.

46. The method of claim 44 wherein the pH indicator is capable of being measured by a means selected from the group consisting of spectrophotometrically, spectrofluorometrically, by luminescence, by reflectance, by electron microscopy, and by radioactivity.

47. The method of claim 46 wherein the pH indicator is capable of being measured spectrofluorometrically, and wherein the marker is measured by fluorescence microscopy.

48. The method of claim 46 wherein the pH indicator is capable of being measured spectrofluorometrically, and wherein the marker is measured by confocal microscopy.

49. A method for screening potential drugs to identify candidate drugs for treating pH-dependent multidrug resistance in mammals comprising:
(a) contacting a mammalian multidrug resistant tumor cell with a potential drug, wherein prior to said contacting it is determined that there is a no defect in the acidification of an intracellular vesicular compartment of the cell; and
(b) determining whether a defect in the acidification of an intracellular vesicular compartment is present in the tumor cell; wherein said defect is symptomatic of the tumor cell being drug-sensitive; and wherein the presence of said defect identifies the potential drug as a candidate drug for the treatment of multidrug resistance.

50. The method of claim 49 further comprising:
(c) contacting a mammalian non-tumorous cell with the candidate drug, wherein prior to said contacting it is determined that there is no defect in the acidification of an intracellular vesicular compartment of the non-tumorous cell; and
(d) determining whether the acidification of the intracellular vesicular compartment of the non-tumorous cell is altered; wherein the lack of an alteration in the acidification of the intracellular vesicular compartment of the non-tumorous cell confirms the identification of the candidate drug.

51. The method of claim 49 wherein an intracellular vesicular compartment of the tumor cell is infiltrated with a pH indicator.

52. The method of claim 49 wherein a plurality of potential drugs are tested at a plurality of drug concentrations.

53. An assay system for screening a potential drug for the treatment of pH-dependent multidrug resistance (MDR) in mammals comprising a mammalian tumor cell susceptible to or experiencing MDR, and a pH indicator that can be placed into an intracellular vesicular compartment of the mammalian tumor cell.

54. A method for treating pH-dependent multidrug resistance in a mammalian tumor cell comprising administering to the tumor cell a pH modulator in an amount effective for disrupting the acidification of an intracellular vesicular compartment of the mammalian tumor cell and thereby alleviating the multidrug resistance in the tumor cell.

55. The method of claim 54 wherein the pH modulator is administered in association with the administration of a chemotherapeutic agent already under administration to the tumor cell.

56. The method of claim 55 wherein said pH modulator is administered simultaneously with said chemotherapeutic agent.

57. The method of claim 56 wherein the pH modulator is administered in a pharmaceutical composition comprising the pH modulator and said chemotherapeutic agent.

58. The method of claim 55 wherein the pH modulator is administered parenterally.

59. The method of claim 55 wherein the pH modulator is administered orally.

60. A therapeutic composition for the treatment of multidrug resistance in a mammal comprising, in unit dose form, a modulator of the pH of a intracellular vesicular compartment and a pharmaceutically acceptable excipient.

61. The composition of claim 60 wherein the composition includes a chemotherapeutic agent to which the mammal has developed said multidrug resistance.
